SEQUENCE LISTING (5 sequences)

SEQ ID NO: 1 - protein, 19 aa, Mycobacterium tuberculosis
DEPLVEVSTDKVDTEIPSP

SEQ ID NO: 2 - DNA, 30 bp, Artificial Sequence
  Description of Artificial Sequence  Primer
gggtagggcatatgacccactatgacgtcg

SEQ ID NO: 3 - DNA, 24 bp, Artificial Sequence
  Description of Artificial Sequence  Primer
gctcgcgctagccgtcatgagccg

SEQ ID NO: 4 - DNA, 26 bp, Artificial Sequence
  Description of Artificial Sequence  Primer
ggagtcaacacatatggccttctccg

SEQ ID NO: 5 - DNA, 22 bp, Artificial Sequence
  Description of Artificial Sequence  Primer
gcgatcggatccacggcgttgg

(19) United States
(12) Patent Application Publication
(10) Pub. No.: US 2003/0190325 A1
(43) Pub. Date: Oct. 9, 2003

(54) Novel peroxiredoxin defense system from mycobacterium tuberculosis

(76) Inventors: Carl F. Nathan, Larchmont, NY (US); Christopher D. Lima, New York, NY (US); Ruslana Bryk, New York, NY (US)

Correspondence Address: Michael L. Goldman, Esq., NIXON PEABODY LLP, Clinton Square, P.O. Box 31051, Rochester, NY, 14603-1051, US

(21) Appl. No.: 10/345,446

(22) Filed: Jan. 15, 2003

Related U.S. Application Data
(60) Provisional application No. 60/348,844, filed on Jan. 16, 2002.

(51) Int. Cl. A61K 39/02

(52) U.S. Cl. 424/190.1

(57) ABSTRACT

The present invention relates to methods of preventing and treating tuberculosis in a subject infected with Mycobacterium tuberculosis. The method involves inhibiting AhpD in the subject under conditions effective to make the pathogen susceptible to antimicrobial reactive nitrogen intermediates or reactive oxygen intermediates. The present invention also relates to methods of preventing and treating tuberculosis in a subject infected with Mycobacterium tuberculosis involving inhibiting dihydrolipoamide dehydrogenase or dihydrolipoamide succinyltransferase in Mycobacterium tuberculosis in the subject under conditions effective to make the pathogen susceptible to antimicrobial reactive nitrogen intermediates or reactive oxygen intermediates. Also disclosed are methods for identifying candidate compounds suitable for treatment or prevention of tuberculosis. Methods of producing an AhpD crystal suitable for X-ray diffraction as well as methods for designing a compound suitable for treatment or prevention of tuberculosis and compounds suitable for treatment or prevention of tuberculosis are also disclosed.

This application claims the benefit of U.S. patent application Ser. No. 60/348,844, filed Jan. 16, 2002, which is hereby incorporated by reference in its entirety.

This invention arose out of research sponsored by the National Institutes of Health, National Heart and Lung Institute (Grant No. HL61241). The U.S. Government may have certain rights in this invention.

### FIELD OF THE INVENTION

The present invention relates to prevention and treatment of tuberculosis in a subject infected with Mycobacterium tuberculosis by inhibiting AhpD, dihydrolipoamide dehydrogenase, and/or dihydrolipoamide succinyltransferase to impart susceptibility to antimicrobial reactive nitrogen intermediates or reactive oxygen intermediates. A method of producing an AhpD crystal suitable for X-ray diffraction and a compound suitable for treatment or prevention of tuberculosis in a subject are also disclosed.

### BACKGROUND OF THE INVENTION

Mycobacterium tuberculosis and Acid Nitrite

Mycobacterium tuberculosis infects about one-third of the human population, persists for decades, and causes disease in a small fraction of those infected. Despite the low disease rate, Mycobacterium tuberculosis is the single leading cause of death from bacterial infection and accounts for an extraordinary proportion of the chronic infectious morbidity and mortality of humankind. Mycobacterium tuberculosis provokes inflammation that leads human macrophages to express the high output isoform of nitric oxide synthase (iNOS or NOS2).

Mycobacterium tuberculosis must cope with reactive nitrogen intermediates (“RNI”) in the context of acid. Mycobacterium tuberculosis is a facultative intracellular parasite of macrophages that encounters RNI and acid (4.5≦pH<7) in the phagolysosome of activated macrophages. Although some mycobacteria frustrate phagosome acidification in nonactivated macrophages (Sturgill-Koszicki ARI 151), activation of the macrophage overcomes this effect and acidification is preserved.

There are two basic clinical patterns that follow infection with Mycobacterium tuberculosis.

In the majority of cases, inhaled tubercle bacilli ingested by phagocytic alveolar macrophages are either directly killed or grow intracellularly to a limited extent in local lesions called tubercles. Infrequently in children and immunocompromised individuals, there is early hematogenous dissemination with the formation of small miliary (millet-like) lesions or life-threatening meningitis. More commonly, within 2 to 6 weeks after infection, cell-mediated immunity develops, and infiltration into the lesion of immune lymphocytes and activated macrophages results in the killing of most bacilli and the walling-off of this primary infection, often without symptoms being noted by the infected individual. Skin-test reactivity to a purified protein derivative (“PPD”) of tuberculin and, in some cases, X-ray evidence of a healed, calcified lesion provide the only evidence of the infection. Nevertheless, to an unknown extent, dormant but viable Mycobacterium tuberculosis bacilli persist.

The second pattern is the progression or breakdown of infection to active disease. Individuals with normal immune systems who are infected with Mycobacterium tuberculosis have a 10% lifetime risk of developing the disease.

In either case, the bacilli spread from the site of initial infection in the lung through the lymphatics or blood to other parts of the body, the apex of the lung and the regional lymph node being favored sites. Extrapulmonary tuberculosis of the pleura, lymphatics, bone, genito-urinary system, meninges, peritoneum, or skin occurs in about 15% of tuberculosis patients. Although many bacilli are killed, a large proportion of infiltrating phagocytes and lung parenchymal cells die as well, producing characteristic solid caseous (cheese-like) necrosis in which bacilli may survive but not flourish. If a protective immune response dominates, the lesion may be arrested, albeit with some residual damage to the lung or other tissue. If the necrotic reaction expands, breaking into a bronchus, a cavity is produced in the lung, allowing large numbers of bacilli to spread with coughing to the outside. In the worst case, the solid tissue, perhaps as a result of released hydrolases from inflammatory cells, may liquefy, which creates a rich medium for the proliferation of bacilli, perhaps reaching 10⁹ per milliliter. The pathologic and inflammatory processes produce the characteristic weakness, fever, chest pain, cough, and, when a blood vessel is eroded, bloody sputum.

RNI Resistance and Medical Importance of New Treatments for Infection by Mycobacterium tuberculosis

RNI generated by NOS2 are essential for the temporary control of tuberculosis in mice (Chan et al., Infect. Immun. 63:736-40 (1995); MacMicking, Proc. Natl. Acad. Sci. USA, 94:5243-48 (1997)). Enzymatically active NOS2 is expressed in the tuberculous human lung within macrophages, the cells ultimately responsible for controlling the infection (Nicholson et al., J. Exp. Med., 183:2293-302 (1996)), and can control the replication of mycobacteria in human pulmonary macrophases in vitro (Nozaki et al., Infect. Immun., 65:3644-47 (1997)). Human macrophages from lungs of patients with tuberculosis release very large amounts of nitric oxide (Wang et al., Eur. Respir. J. 11:809-815 (1998)). Surgical specimens of human lungs from a total of 28 different subjects with tuberculosis have been studied for NOS2 expression in three independent studies from Italian, American, and Ethiopian plus Swedish study centers. In all 28 specimens, NOS2 was abundantly expressed in the tuberculous lesions (Facchetti et al., Am. J. Pathol., 154:145-152 (1999); Chen et al., Am. J. Resp. Crit. Care Med., 166:178 (2002); Schön, Dissertation, No. 749, Linköping Universitet (2002)).

Despite the evidence that (i) NOS2 is expressed in macrophages at the sites of tuberculosis, (ii) that NOS2 is essential for control of tuberculosis and (iii) that RNI produced by NOS2 are involved in the killing of M. tuberculosis within macrophages (Erht et al., J. Exp. Med., 194:1123-1140 (2001)), nonetheless some viable M. tuberculosis organisms appear to persist lifelong in a large portion of people who have become infected. At any time thereafter, these persistent bacteria may resume replication and cause disease. This combination of circumstances strongly suggests that M. tuberculosis expresses mechanisms of RNI resistance. If these mechanisms of RNI resistance were inhibited by pharmacologic agents, persons infected with M. tuberculosis might be able to eradicate the organism through the actions of their immune response, the immune response normally including the expression of NOS2. Such eradication of otherwise persistent M. tuberculosis would be expected to have the following beneficial effects: helping treat tuberculosis, which is now estimated to take the lives of about 8 million people a year; helping prevent tuberculosis in individuals who are subclinically infected, who are currently estimated to comprise about one-third of the world's population; and by the first two actions, helping to interrupt the pandemic of tuberculosis, that is, reducing the likelihood of its transmission to new hosts. In addition, such a pharmacologic approach, being fundamentally distinct in its mechanism of action from all existing anti-tuberculosis chemotherapy, would be expected to be equally effective against strains of M. tuberculosis that are currently drug-sensitive and those that are already resistant to multiple drugs.

Identificatiion of a Mechanism of RNI Resistance in M. tuberculosis: the Peroxynitrite Reductase Activity of AhpC from M. tuberculosis

For Mycobacterium tuberculosis, the rapid emergence of multidrug resistance is associated with mortality rates near 50% even in optimally treated patients with mycobacterial disease. The intersection of the tuberculosis pandemic with the HIV epidemic threatens even higher rates of active tuberculosis in the infected population, which in turn may increase the rate of infection among all people in contact, regardless of their medical or economic status. New anti-tuberculous drugs are urgently needed.

Alkyl hydroperoxide reductase was first cloned and purified from S. typhimurium and E. coli as the product of genes induced by oxidative stress under the positive control of the oxyR gene (Storz et al., J. Bacteriol. 181:2049-55 (1989); Jacobson et al., J. Biol. Chem., 264:1488-96 (1989); Tartaglia et al., J. Biol. Chem., 265:10535-40 (1990)). Hydroperoxides are mutagenic in bacteria (Farr, Microbiol. Rev., 55:561-85 (1991)). Overexpression of alkyl hydroperoxide reductase activity suppressed spontaneous mutagenesis associated with aerobic metabolism in oxyR mutants of S. typhimurium and E. coli (Storz et al., Proc. Natl. Acad. Sci. USA, 84:917-21 (1987); Greenberg, EMBO J., 7:2611-17 (1988)). The isolated enzyme uses NAD(P)H to reduce alkyl hydroperoxides to the corresponding alcohols. This activity is manifest by a tetramer comprised of two 57-kDa monomers of the NAD(P)H-oxidizing flavoprotein AhpF, and two 21-kDa monomers of its peroxide-reducing partner, AhpC. Only a few homologs of AhpF have been identified (Chae et al., Proc. Natl. Acad. Sci. USA, 91:7017-21 (1994)). In contrast, AhpC homologs are widely distributed among prokaryotes (Chae et al., J. Biol. Chem., 269:27670-678 (1994)), and AhpC is ˜40% identical to thioredoxin peroxidase from yeast (Chae et al., Proc. Natl. Acad. Sci. USA, 91:7017-21 (1994)), rat (Chae et al., Proc. Natl. Acad. Sci. USA, 91:7017-21 (1994)), plants amoebae, nematodes, rodents, and humans (Chae et al., Proc. Natl. Acad. Sci. USA, 91:7017-21 (1994); Lim et al., Gene, 140:279-84 (1994); Jin et al., J. Biol. Chem., 272:30952-61 (1997)). Therefore, homologs of AhpC define a large family of antioxidants present in organisms from all kingdoms.

Mycobacterium tuberculosis alkyl hydroperoxide reductase C (AhpC), a member of the peroxiredoxin family of non-heme peroxidases, protects heterologous bacterial and human cells against oxidative and nitrosative injury (Storz et al., J. Bacteriol., 171: 2049 (1989); Chen et al., Mol. Cell., 1: 795 (1998)). The redundancy of peroxiredoxins in Mycobacterium tuberculosis complicates interpretation of the phenotype of an ahpC-deficient mutant (Springer et al., Infect. Immun., 69: 5967 (2001)). AhpC metabolizes peroxides (Ellis et al., Biochemistry 36: 13349 (1997)) and peroxynitrite (Bryk et al., Nature, 407: 211 (2000)) via a conserved N-terminal cysteine residue, which undergoes oxidation. To complete the catalytic cycle, the cysteine residue must again be reduced. Various peroxiredoxins rely on diverse reducing systems, including AhpF; thioredoxin and thioredoxin reductase; tryparedoxin, trypanothione and trypanothione reductase; and cyclophilin (e.g. Lee et al., J. Biol. Chem., 276: 29826 (2001)). It is not known what serves as an AhpC reductase in Mycobacterium tuberculosis. The genome of Mycobacterium tuberculosis H37Rv encodes no AhpF-like proteins (Cole et al., Nature, 393: 537 (1998)). Mycobacterium tuberculosis thioredoxin reductase and thioredoxin did not support the activity of AhpC (Hillas et al., J. Biol. Chem., 275: 18801 (2000)). The Mycobacterium tuberculosis ahpC gene lies 11 nucleotides upstream of a coding region denoted ahpD based on an apparent bicistronic operon with ahpC. Recombinant AhpD functions as a weak peroxidase, but does not appear to interact with AhpC physically or functionally (Hillas et al., J. Biol. Chem., 275: 18801 (2000)).

The present invention is directed to overcoming these deficiencies in the art.

### SUMMARY OF THE INVENTION

The present invention relates to a method of preventing onset of tuberculosis in a subject infected with Mycobacterium tuberculosis. The method involves inhibiting AhpD in the subject under conditions effective to make the pathogen susceptible to antimicrobial reactive nitrogen intermediates or reactive oxygen intermediates.

The present invention also relates to a method of treating tuberculosis in a subject. The method involves inhibiting AhpD in the subject under conditions effective to make the pathogen susceptible to antimicrobial reactive nitrogen intermediates or reactive oxygen intermediates.

Another aspect of the present invention relates to a method of preventing onset of tuberculosis in a subject infected with Mycobacterium tuberculosis. The method involves inhibiting dihydrolipoamide dehydrogenase in Mycobacterium tuberculosis in the subject under conditions effective to make the pathogen susceptible to antimicrobial reactive nitrogen intermediates or reactive oxygen intermediates.

Yet another aspect of the present invention relates to a method of treating tuberculosis in a subject. The method involves inhibiting dihydrolipoamide dehydrogenase in Mycobacterium tuberculosis in the subject under conditions effective to make the pathogen susceptible to antimicrobial reactive nitrogen intermediates or reactive oxygen intermediates.

The present invention also relates to a method of preventing onset of tuberculosis in a subject infected with Mycobacterium tuberculosis. The method involves inhibiting dihydrolipoamide succinyltransferase in Mycobacterium tuberculosis in the subject under conditions effective to make the pathogen susceptible to antimicrobial reactive nitrogen intermediates or reactive oxygen intermediates.

Another aspect of the present invention relates to a method of treating tuberculosis in a subject. The method involves inhibiting dihydrolipoamide succinyltransferase in Mycobacterium tuberculosis in the subject under conditions effective to make the pathogen susceptible to antimicrobial reactive nitrogen intermediates or reactive oxygen intermediates.

Yet another aspect of the present invention relates to a method of producing an AhpD crystal suitable for X-ray diffraction. The method first involves subjecting a solution of AhpD under conditions effective to grow a crystal of AhpD to a size suitable for X-ray diffraction. Then, an AhpD crystal suitable for X-ray diffraction is obtained.

The present invention also relates to a method for identifying candidate compounds suitable for treatment or prevention of tuberculosis in a subject. The method first involves contacting AhpD with a compound. Then, those compounds which bind to the AhpD are identified as candidate compounds suitable for treatment or prevention of tuberculosis in a subject.

Another aspect of the present invention relates to a method for identifying candidate compounds suitable for treatment or prevention of tuberculosis in a subject. The method first involves contacting a dihydrolipoamide dehydrogenase in Mycobacterium tuberculosis with a compound. Then, those compounds which bind to the dihydrolipoamide dehydrogenase in Mycobacterium tuberculosis are identified as candidate compounds suitable for treatment or prevention of tuberculosis in a subject.

Another aspect of the present invention relates to a method for identifying candidate compounds suitable for treatment or prevention of tuberculosis in a subject. The method first involves contacting a dihydrolipoamide succinyltransferase in Mycobacterium tuberculosis with a compound. Then, those compounds which bind to the dihydrolipoamide succinyltransferase in Mycobacterium tuberculosis are identified as candidate compounds suitable for treatment or prevention of pathogen infection in a subject.

The present invention also relates to a method for designing a compound suitable for treatment or prevention of tuberculosis in a subject. The method first involves providing a three-dimensional structure of a crystallized AhpD. Then, a compound having a three-dimensional structure which will bind to one or more molecular surfaces of the AhpD is designed.

Another aspect of the present invention relates to a compound suitable for treatment or prevention of tuberculosis in a subject. The compound has a three-dimensional structure which will bind to one or more molecular surfaces of the AhpD having a three dimensional crystal structure defined by the atomic coordinates set forth in FIG. 1.

The present invention ascribes new functions to AhpD, dihydrolipoamide dehydrogenase (Lpd), and dihydrolipoamide succinyltransferase (SucB), each of which supports the antioxidant defense of M. tuberculosis and holds interest as a drug target for tuberculosis. The AhpD crystal structure at 2.0 Å resolution reveals a trimer whose protomers display a unique fold that contains a thioredoxin-like active site that is responsive to lipoic acid. Lpd, SucB (the sole lipoyl protein detected in M. tuberculosis), AhpD, and alkylhydroperoxide reductase subunit C (AhpC) together comprise an NADH-dependent peroxidase and peroxynitrite reductase. AhpD represents a new class of thioredoxin-like molecules that enables a novel antioxidant defense. If SucB or Lpd could be inhibited in M. tuberculosis without affecting their human counterparts, the Krebs cycle in M. tuberculosis as well as the bacillus' ability to synthesize acetyl CoA could both be vulnerable. Acetyl CoA is essential for the glyoxylate shunt that helps sustain persistence of M. tuberculosis (McKinney et al., Nature, 406: 735 (2000), which is hereby incorporated by reference in its entirety) and for formation of the fatty acid-rich cell wall, which constitutes both a barrier and target for chemotherapy.

The present invention is the first known instance in which essential metabolic enzymes also support antioxidant defenses. The α-keto acid substrates of these enzymes can also provide antioxidant defense (O'Donnell et al., J. Exp. Med., 165: 500 (1987), which is hereby incorporated by reference in its entirety).

### BRIEF DESCRIPTION OF THE DRAWINGS

FIG. 1 sets forth the atomic coordinates that defines the three-dimensional crystal structure of AhpD.

FIGS. 2A-C show the AhpD crystal structure. FIG. 2A illustrates a ribbon diagram of AhpD trimer. Helices are denoted by tubes designated a (numbered from N- to C-terminus) and connecting peptides by ribbons. Three monomers are shown. Cys130 and Cys133 are best seen on α7 of one of the protomers. Graphics were prepared using SETOR (Evans, J. Molec. Graph., 11: 134 (1993), which is hereby incorporated by reference in its entirety). FIG. 2B shows structure-based least-squares sequence alignment between active site cysteines and helices in AhpD and thioredoxins from E. coli (PDB Accession No. 2TRX) and T4 bacteriophage (PDB Accession No. 1AAZ). The shaded boxes highlight the di-cysteine motif. FIG. 2C is a ligand-binding molecular surface representation of AhpD, produced with GRASP (Nicholls et al., Proteins: Struct. Funct. Genet., 11: 281 (1991), which is hereby incorporated by reference in its entirety). The orientation is similar to FIG. 2A. Three protomers are shown. Also shown are Cys130 and Cys133.

FIGS. 3A-C illustrate representative surfaces of AhpD that surround the active site cysteine residue, Cys 130, which can be targeted for potential inhibitor interactions. FIG. 3A shows a surface of AhpD with the Cys130 residue shaded. FIG. 3B shows a surface of AhpD with a surface scribed around the Cys130 residue. FIG. 3C shows a surface of AhpD with the scribed surface around the Cys130 filled in.

FIGS. 4A-C illustrate representative surfaces of AhpD that surround the active site cysteine residue, Cys 133, which can be targeted for potential inhibitor interactions. FIG. 4A shows a surface of AhpD with the Cys133 residue shaded. FIG. 4B shows a surface of AhpD with a surface scribed around the Cys133 residue. FIG. 4C shows a surface of AhpD with the scribed surface around the Cys133 filled in.

FIG. 5 depicts mycobacterial lysates supporting AhpC peroxidase activity only in the presence of AhpD. Reaction mixtures (0.5 ml) contained 50 mM potassium phosphate (KPi) pH 7.0, 1 mM EDTA, 200 μM NADH, 5 μM recombinant AhpC, 10 μM recombinant AhpD and 50 μl (3.8 mg/ml) M. tuberculosis H37Rv lysate (▪). Reactions were initiated by addition of 0.5 mM H₂O₂ and consumption of NADH was followed over time by A₃₄₀. Control reactions were carried out with no AhpC (□), no AhpD (Δ), no lysates (◯), or 200 μM NADPH (●) instead of NADH.

FIGS. 6A-D show the identification of Lpd (Rv0462) and SucB (Rv2215) as components of the AhpC/AhpD-dependent peroxidase system. FIG. 6A depicts partial purification of Lpd from M. tuberculosis H37Rv. Samples were tested as in FIG. 5, analyzed by 10% SDS-PAGE and stained with Coomassie. Lane 1, lysate; lane 2, 0-30% (NH₄)₂SO₄ precipitate with no activity; lane 3, 30-70% active (NH₄)₂SO₄ precipitate; lane 4, activity peak from Q Sepharose. FIG. 6B shows the elution profile from Q Sepharose. The bar diagram shows the peak activity profile of fractions whose Coomassie-stained 10% SDS-PAG electrophoregram is displayed below. FIG. 6C shows the identification of lipoylated proteins in mycobacterial lysates. Samples were run on 10% SDS-PAGE, transferred to nitrocellulose and western blotted with anti-lipoic acid Ab (1:10,000). Lane 1, M. tuberculosis H37Rv lysate; lane 2, M. bovis BCG lysate; lane 3, active peak after Q Sepharose. FIG. 6D illustrates that M. tuberculosis H37Rv lysates depleted of the single lipoylated protein no longer support AhpC peroxidatic activity. L, lysates (50 μg); B, immune complexes on beads (12.5 μl); S, supernates (50 μg) after removing the beads. Results are expressed as a percentage of starting activity in lysates (100%). Three cycles of IPs (IP-1, IP-2, IP-3) led to complete depletion.

FIGS. 7A-C show reconstitution of AhpC enzymatic activity with recombinant proteins. FIG. 7A shows recombinant proteins produced in E. coli. Shown are final pure preparations of proteins analyzed by 15% or 10% SDS-PAGE and visualized with Coomassie stain. Lane 1, AhpC (2.5 μg); lane 2, AhpD (5 μg); lane 3, Lpd (1 μg); lane 4, SucB (10 μg). FIG. 7B shows that recombinant AhpD, SucB, and Lpd reconstitute AhpC peroxidase activity. Reaction mixtures (0.5 ml) contained 50 mM KPi, pH 7.0, 1 mM EDTA, 150 μM NADH, 0.5 μM AhpC, 2 pM AhpD, 2 μM SucB and 0.5 μM Lpd (▪). Reactions were initiated by addition of 0.5 mM H₂O₂ and consumption of NADH was followed over time by A₃₄₀. Control reactions were carried out with no Lpd (●), no SucB (□), no AhpC (▴) or 2 μM AhpD C130S (◯) instead of AhpD. FIG. 7C shows that recombinant AhpD, SucB and Lpd reconstitute AhpC peroxynitrite reductase activity during steady-state infusion of peroxynitrite. Reaction mixtures (1.5 ml) contained 100 mM KPi pH 7.0, 100 μM DTPA, 100 μM dihydrorhodamine, 50 μM NADH and either no protein (▪), 2 μM AhpC and 5 μM recombinant S. typhimurium AhpF (□) or 2 μM AhpC, 5 μM AhpD, 5 μM SucB and 5 μM Lpd (●).

### DETAILED DESCRIPTION OF THE INVENTION

The present invention relates to a method of preventing onset of tuberculosis in a subject infected with Mycobacterium tuberculosis. The method involves inhibiting AhpD in the subject under conditions effective to make the pathogen susceptible to antimicrobial reactive nitrogen intermediates or reactive oxygen intermediates. AhpD refers to the protein encoded by the ahpD (Rv2429; NCBI# 15609566) gene in Mycobacterium tuberculosis or any functional homolog. The ahpD gene was so named for being located adjacent to the ahpC gene, which encodes alkylhydroperoxide reductase subunit C (AhpC), on the chromosome of M. tuberculosis. The complete genome sequence of M. tuberculosis, H37Rv, and sequences and annotations for the various genes (including Rv2429, Rv0462, and Rv2215) have been deposited and are disclosed in EMBL/GenBank/DDBJ as MTBH37RV, accession number AL123456 (Cole et al., Nature, 393: 537-544 (1998); http://www.sanger.ac.uk/Projects/M_tuberculosis/, which are hereby incorporated in their entirety).

AhpD is both structurally novel and narrowly distributed. All proteins in the thioredoxin superfamily (thioredoxins, glutaredoxins, tryparedoxin, and the Dsb family) share a common fold typically comprised of a 4-stranded β-sheet and 3 flanking α-helices (Katti et al., J. Mol. Biol., 212: 167 (1990), which is hereby incorporated by reference in its entirety). In contrast, AhpD shares only the C-terminal signature motif, Cys-X—X-Cys, disposed as in thioredoxin but within a novel fold. AhpD homologs have been identified only in mycobacteria, Streptomycetes and a few proteobacteria such as Bradyrhizobium and Caulobacter.

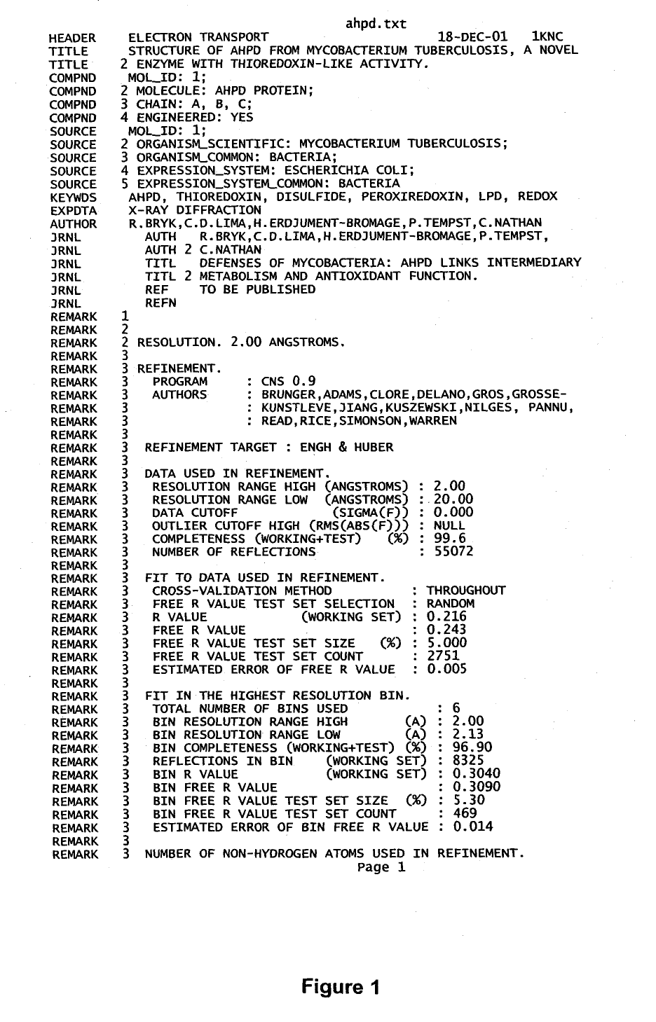

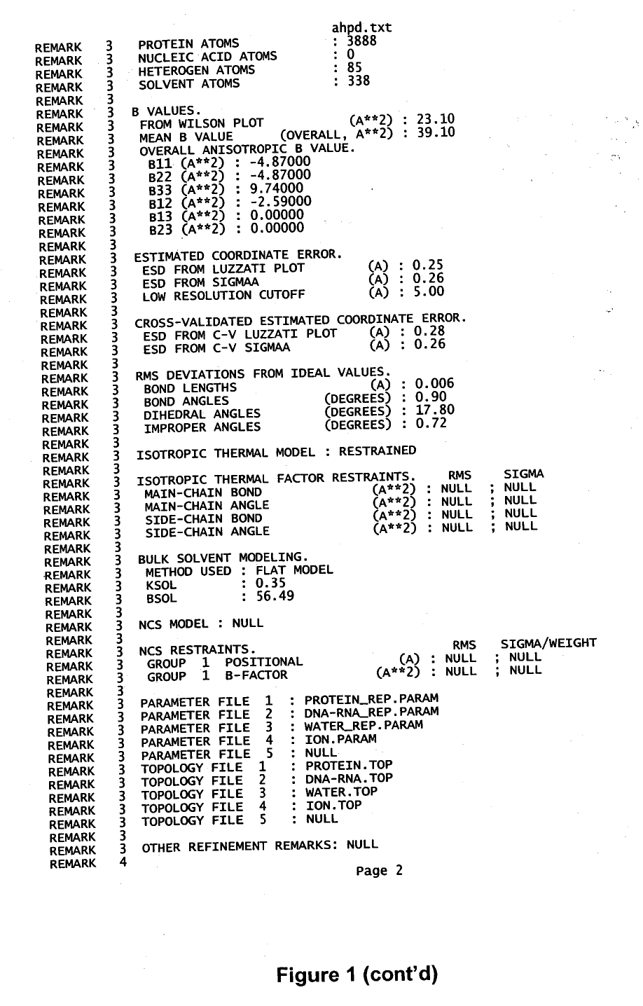

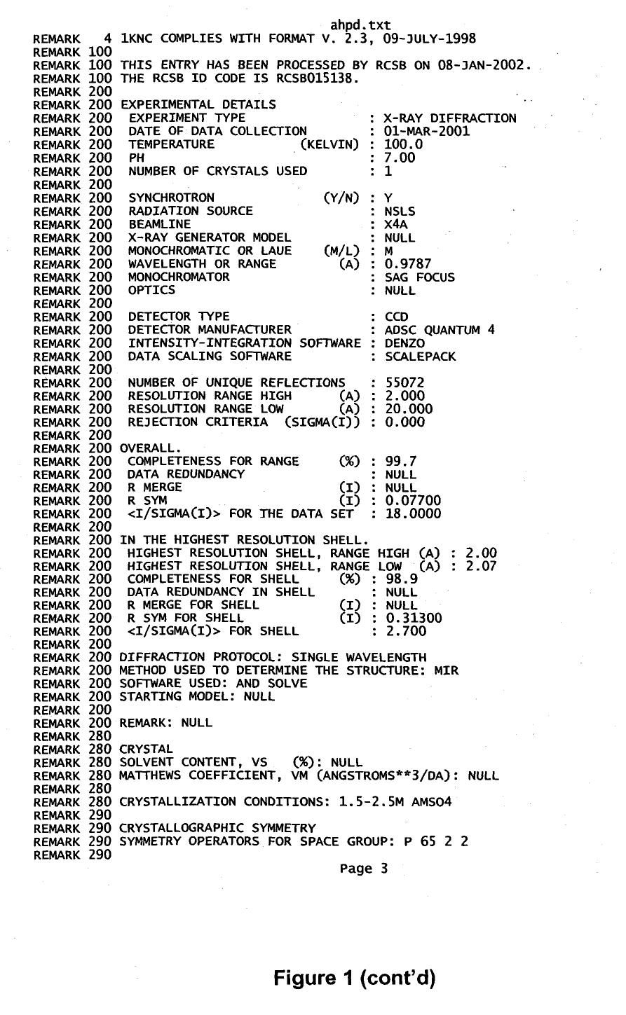

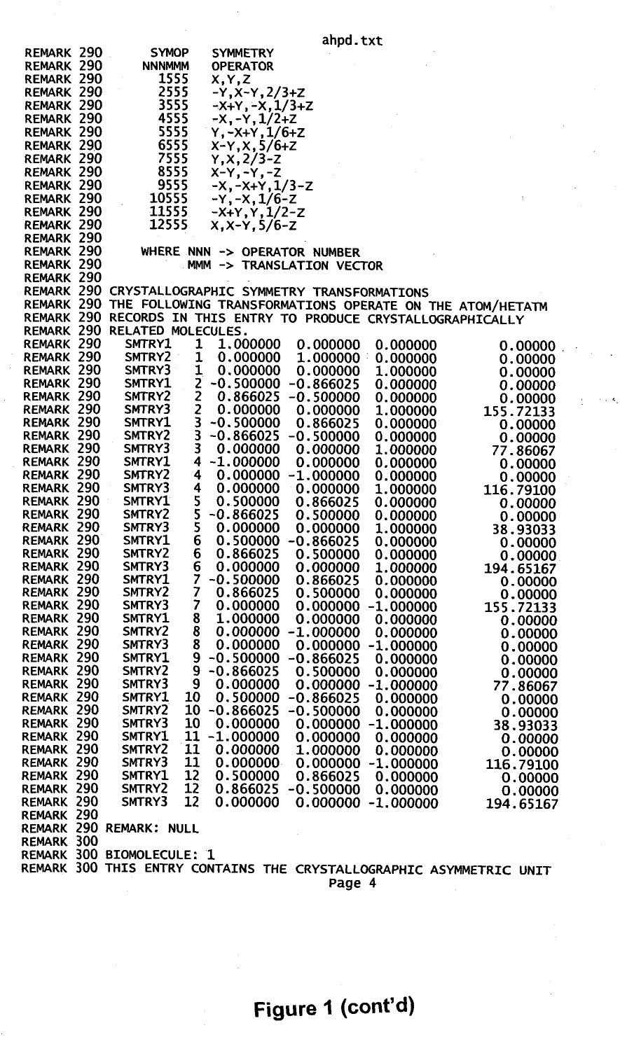

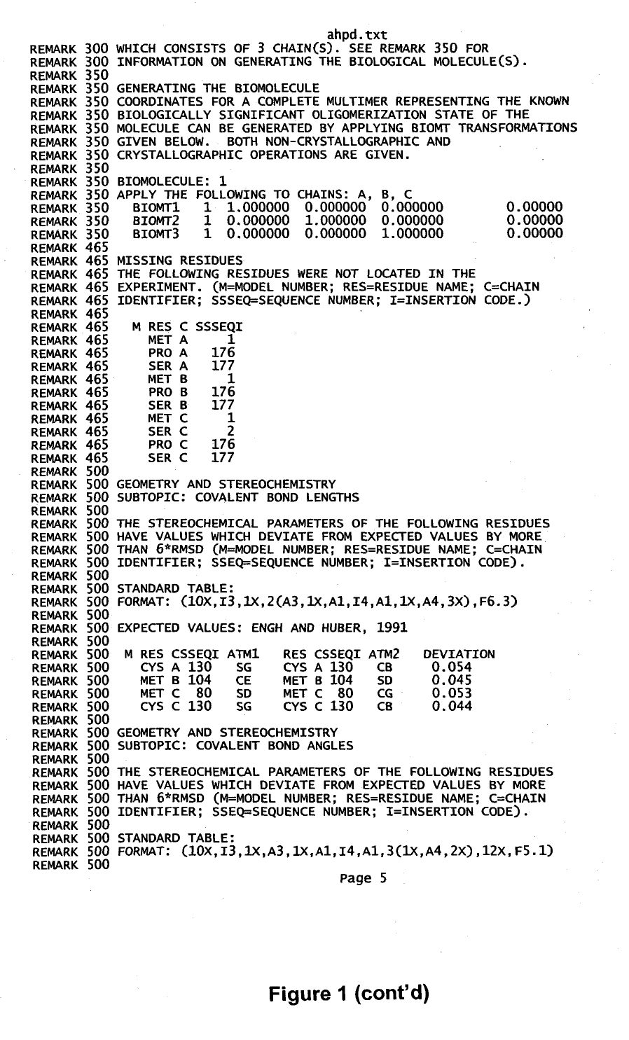

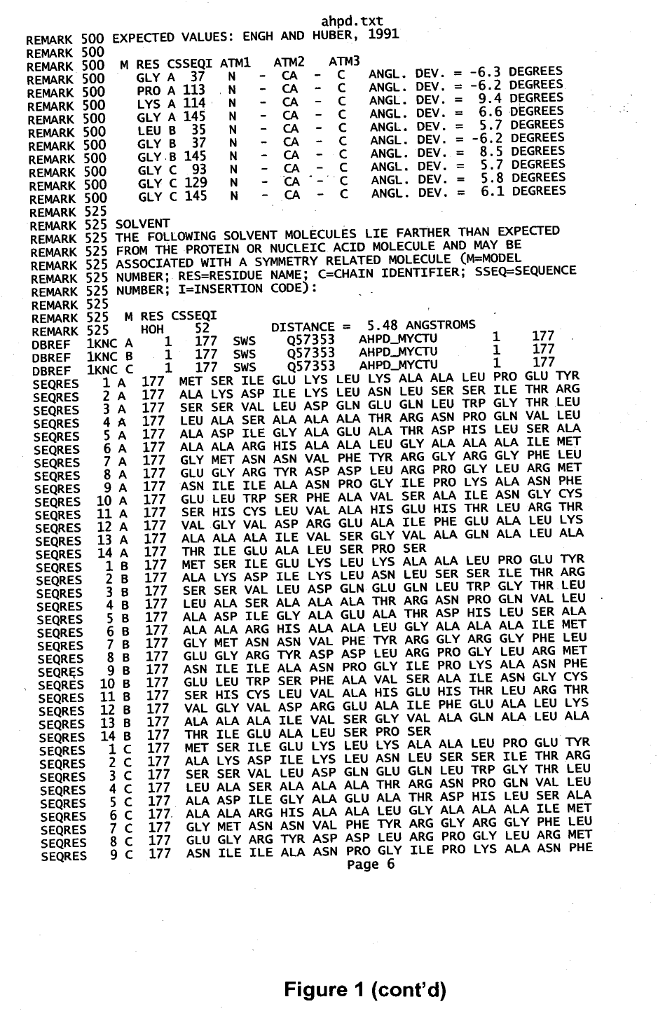

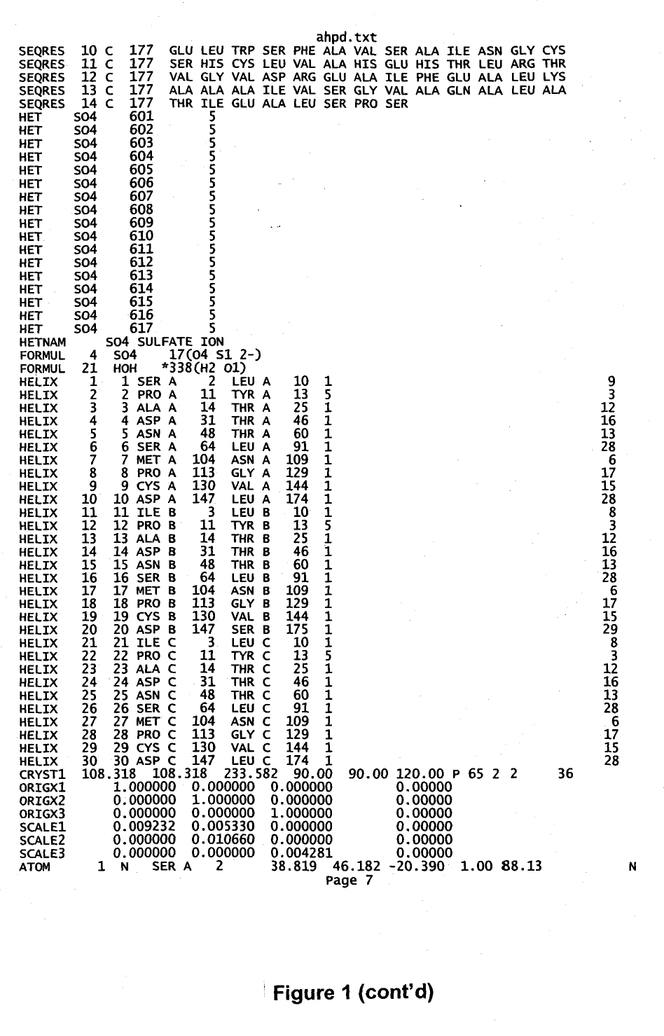

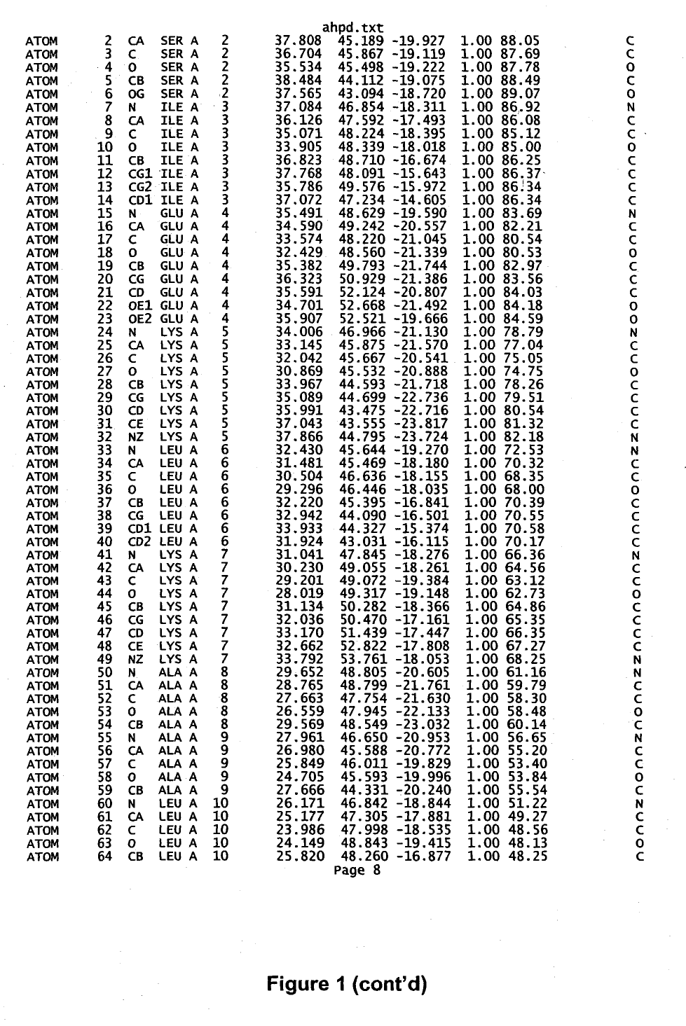

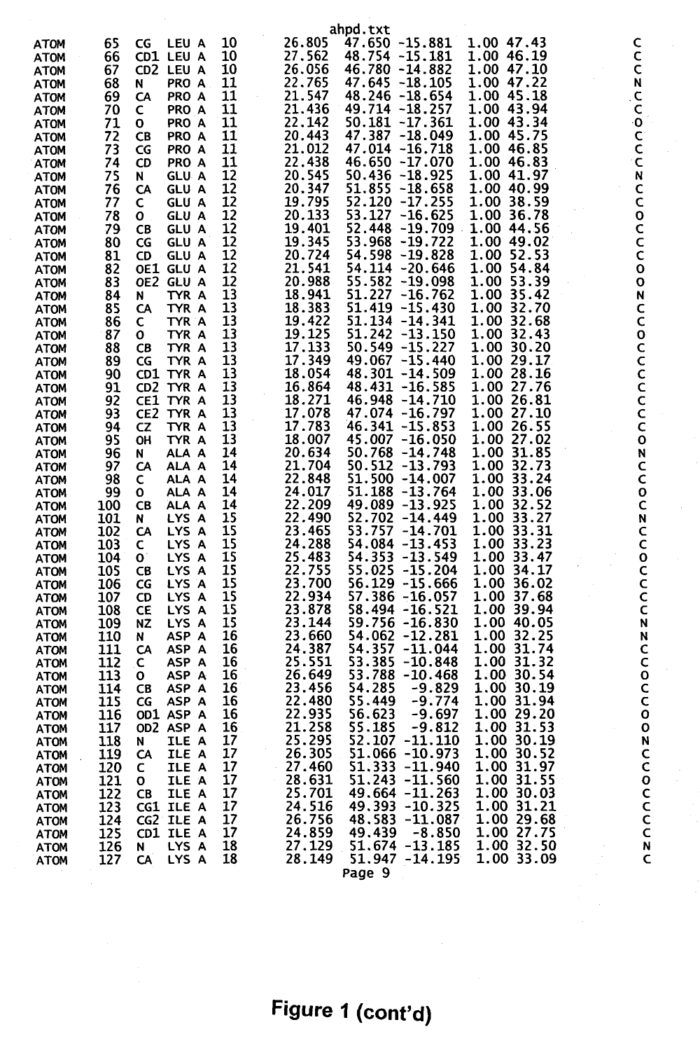

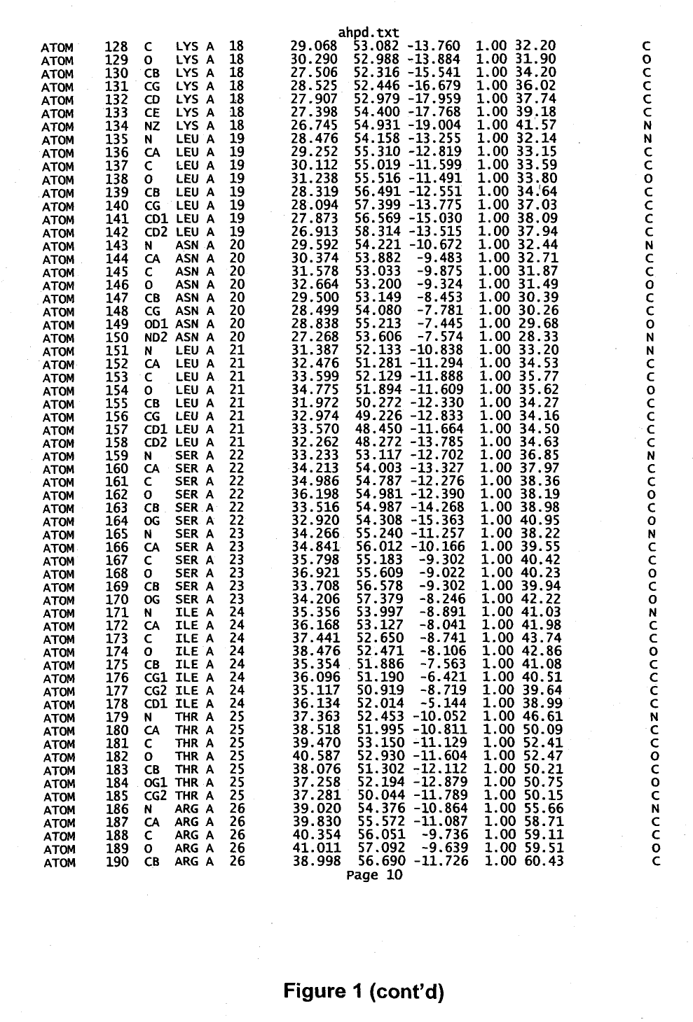

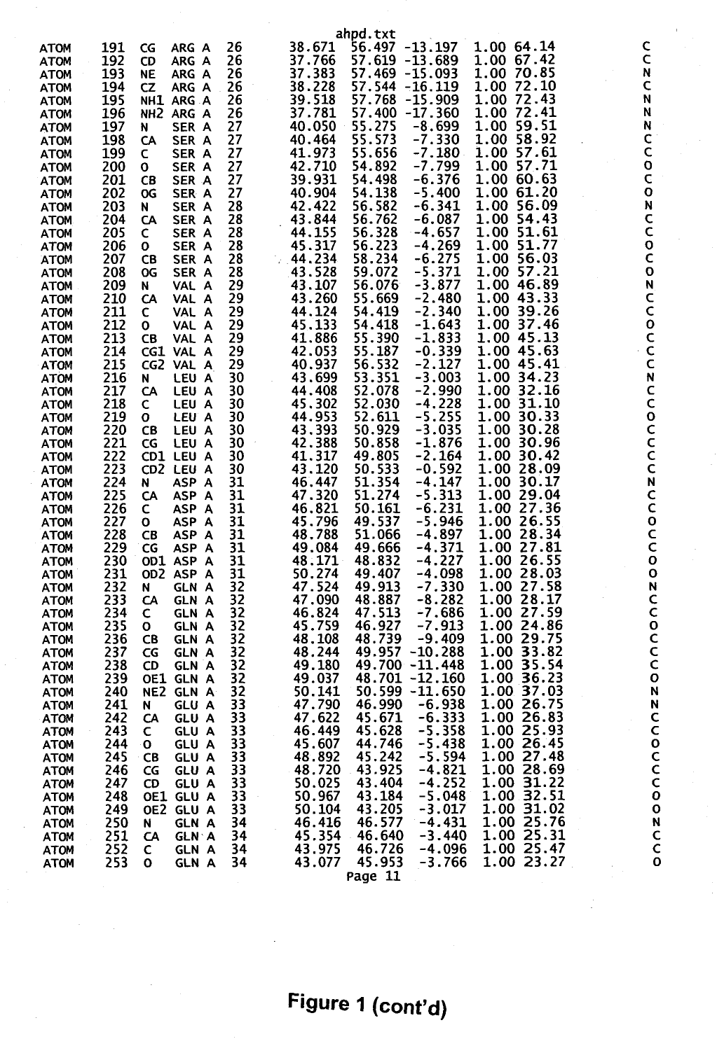

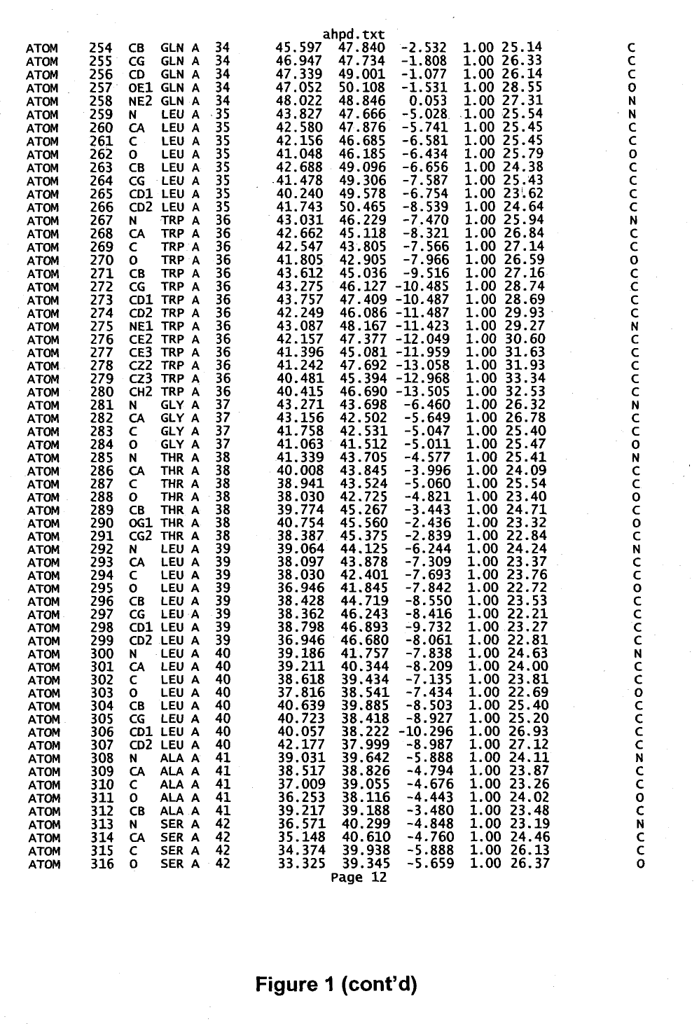

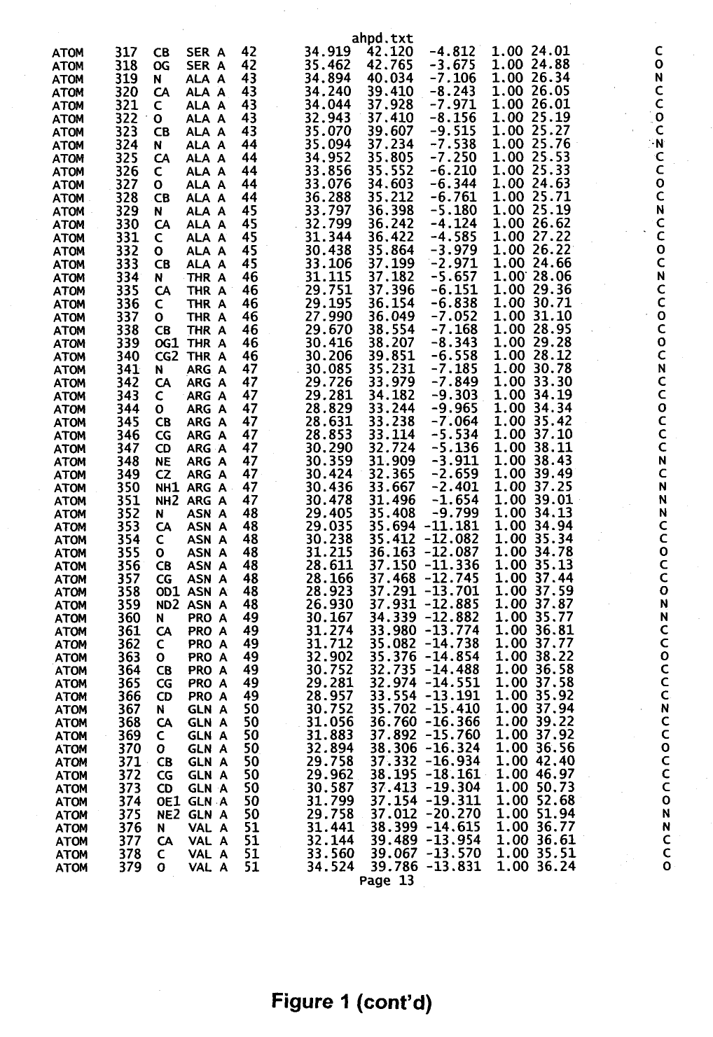

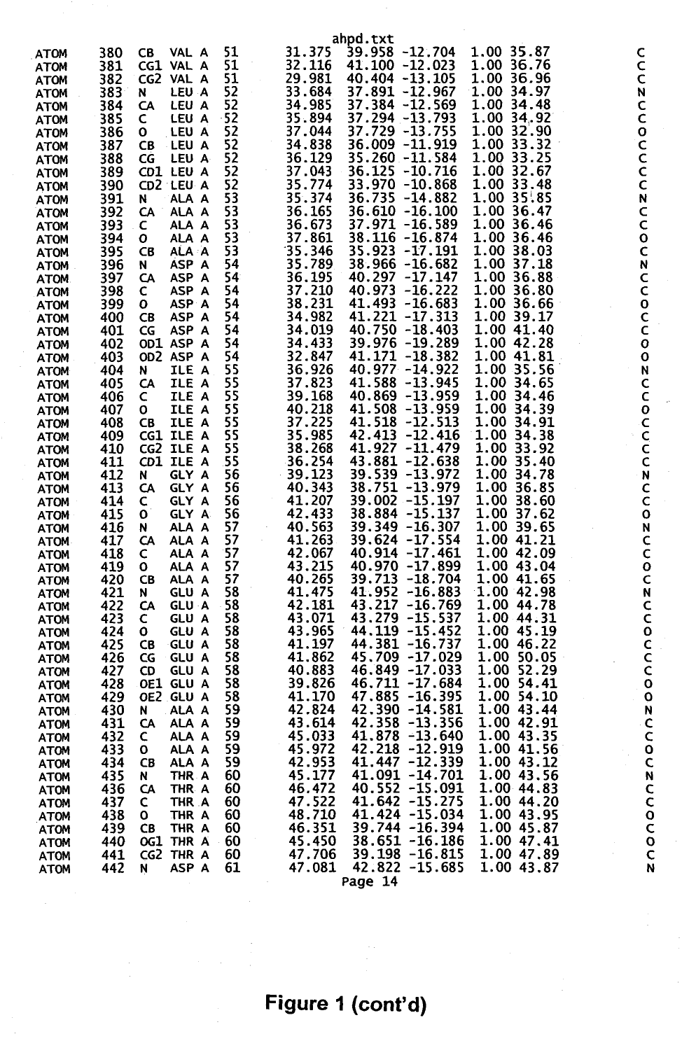

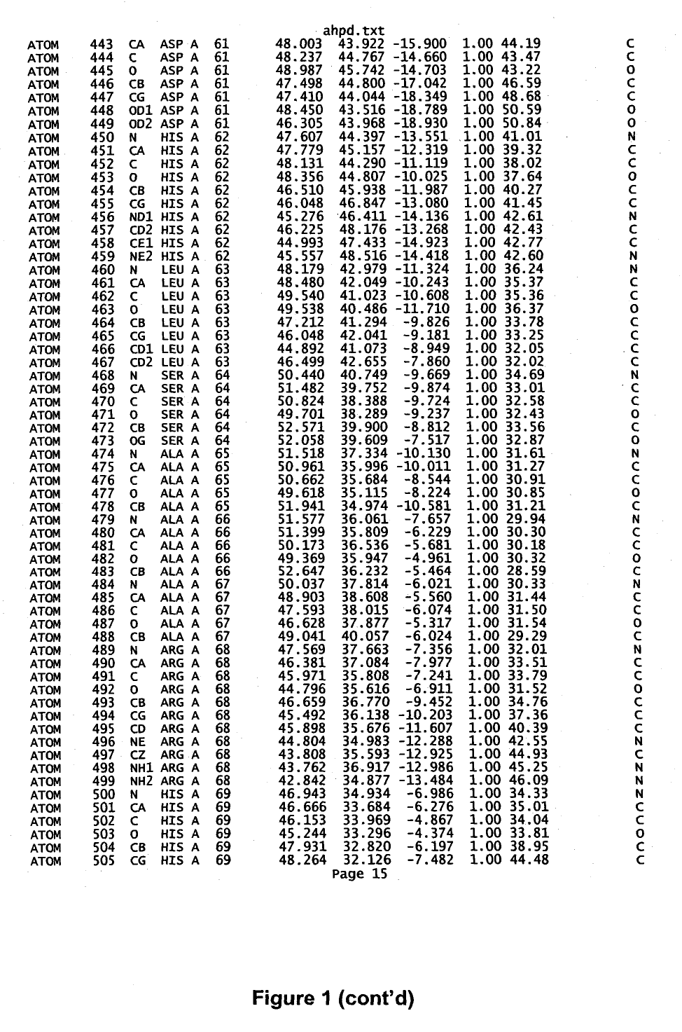

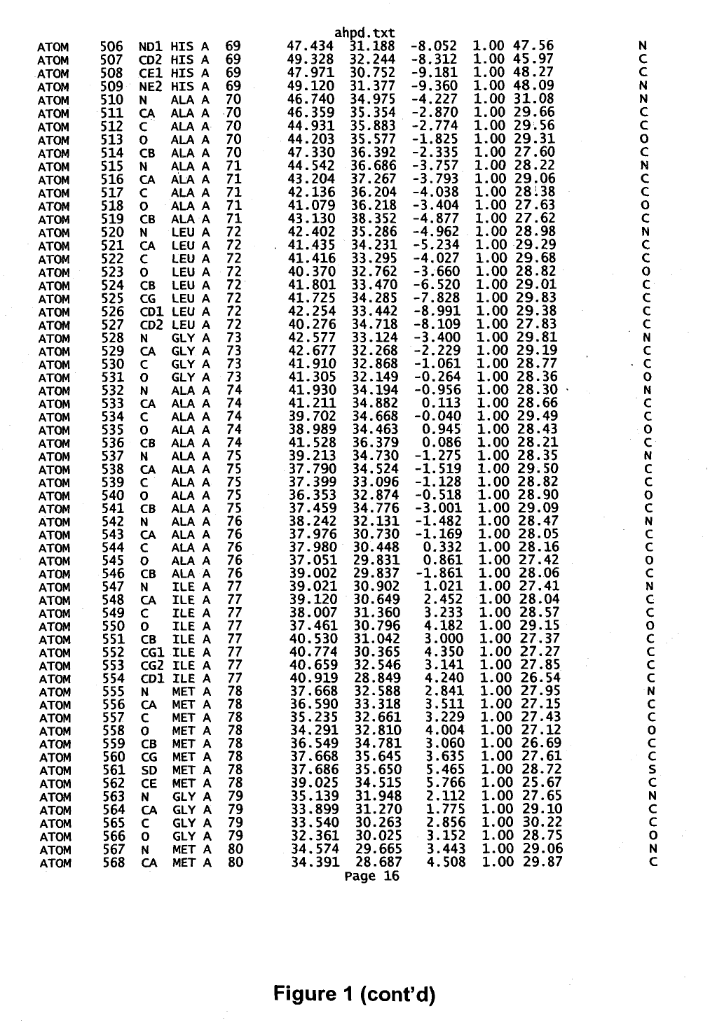

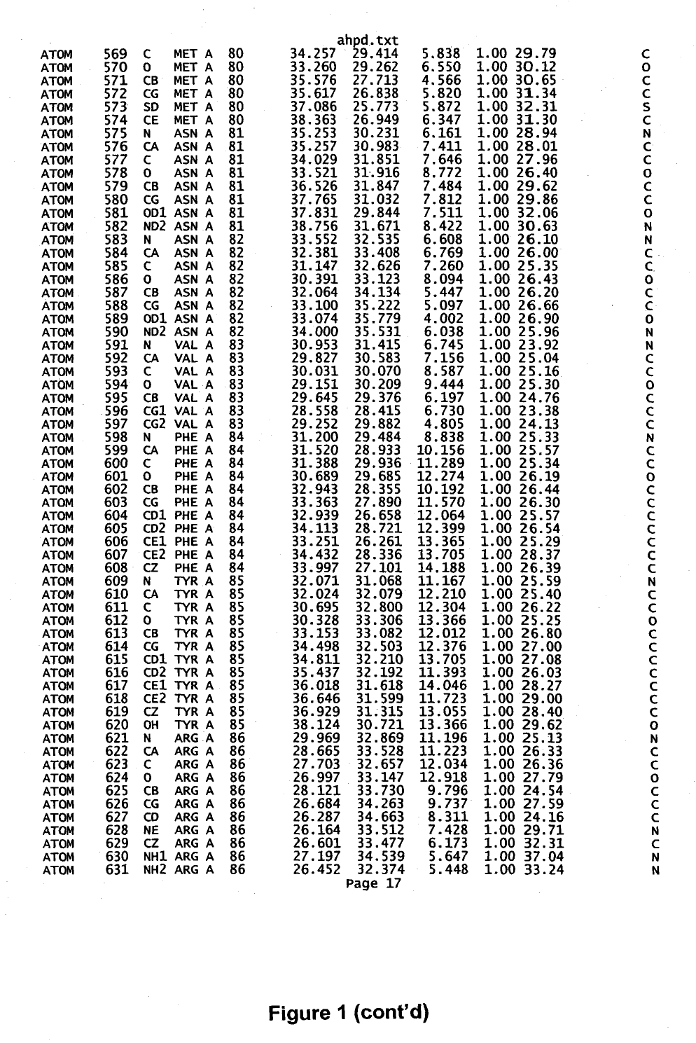

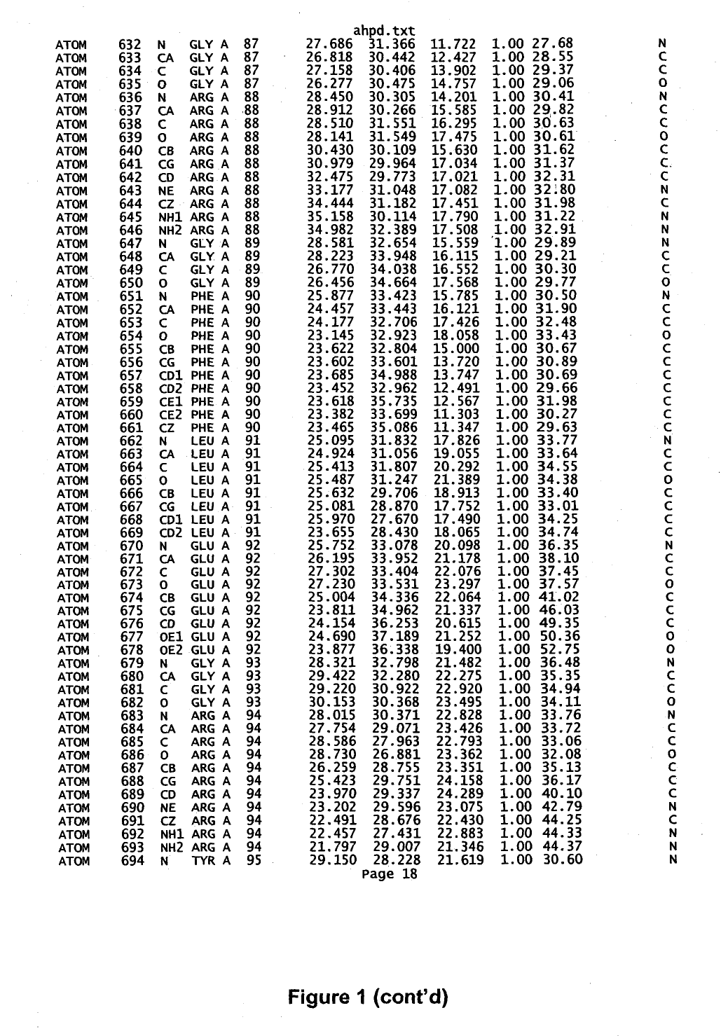

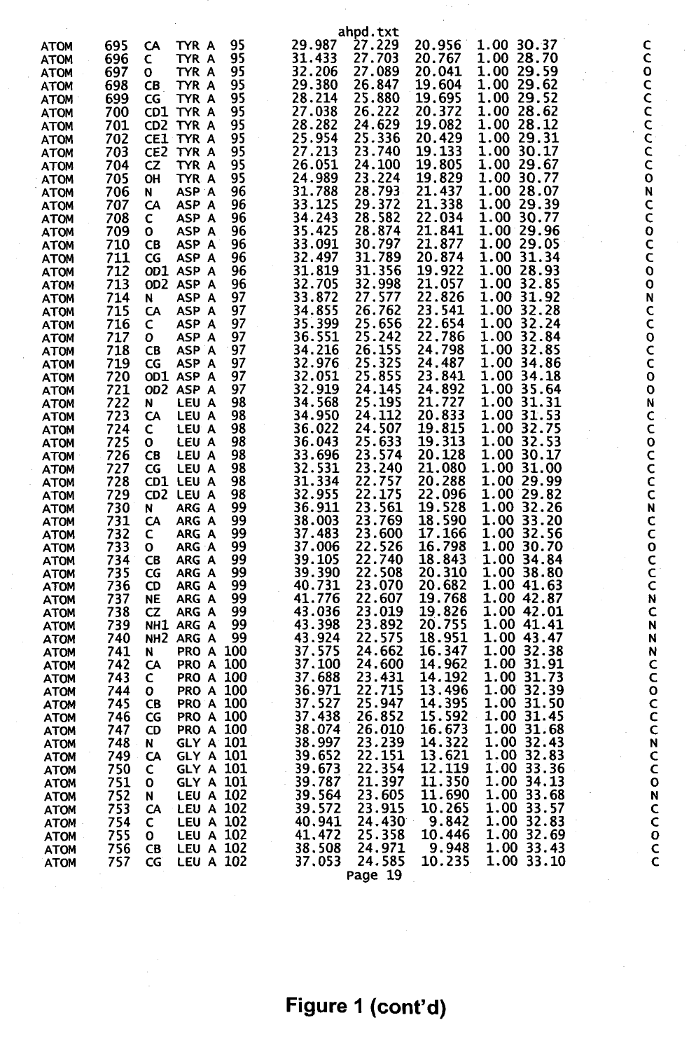

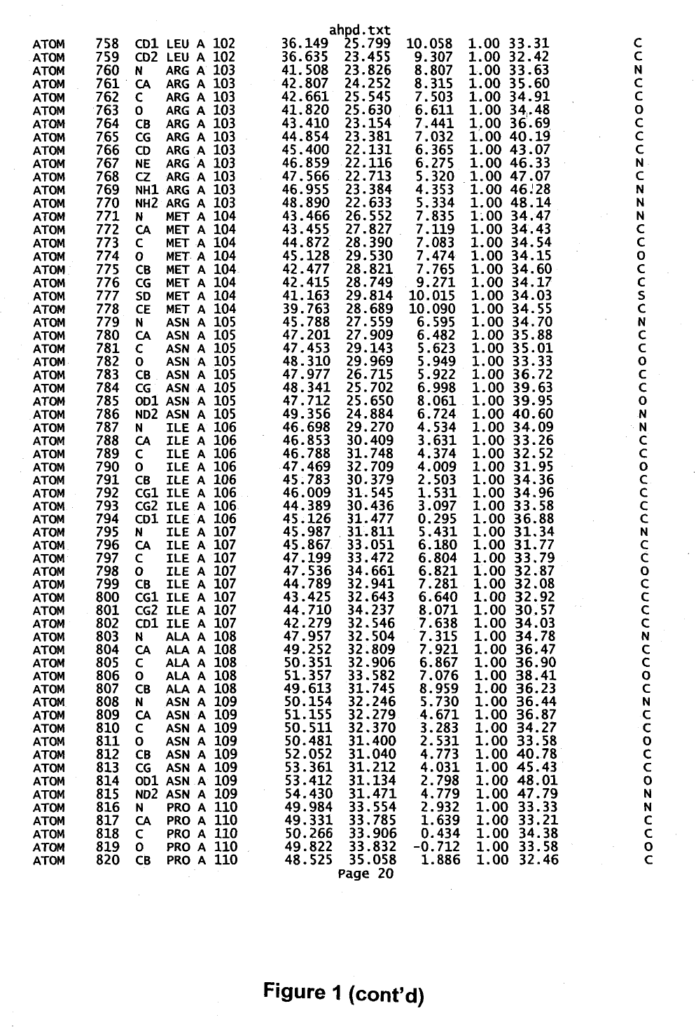

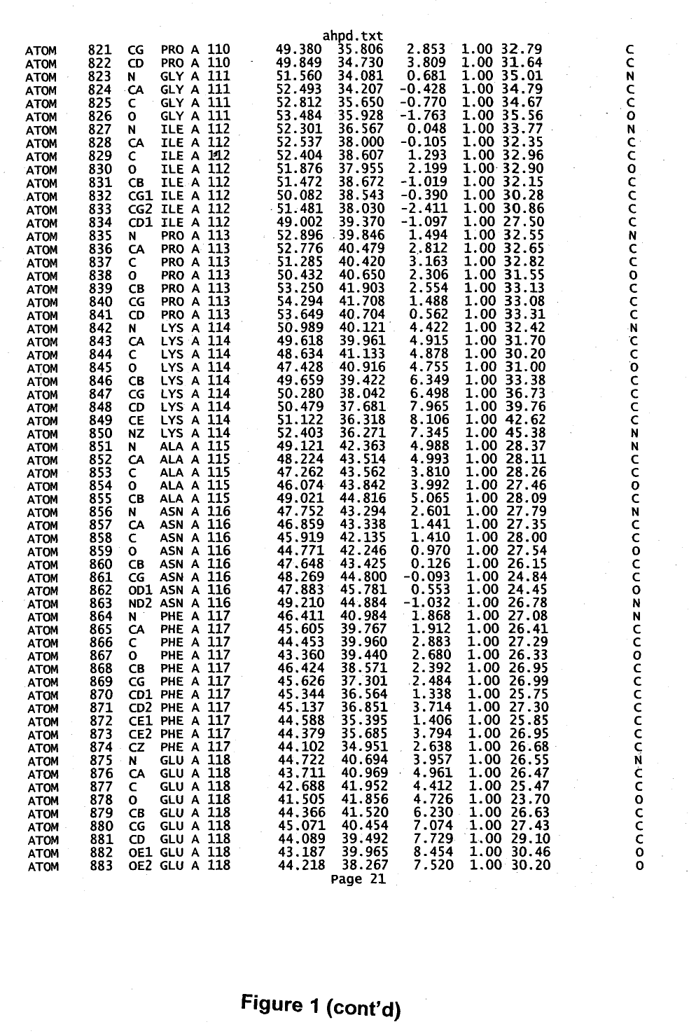

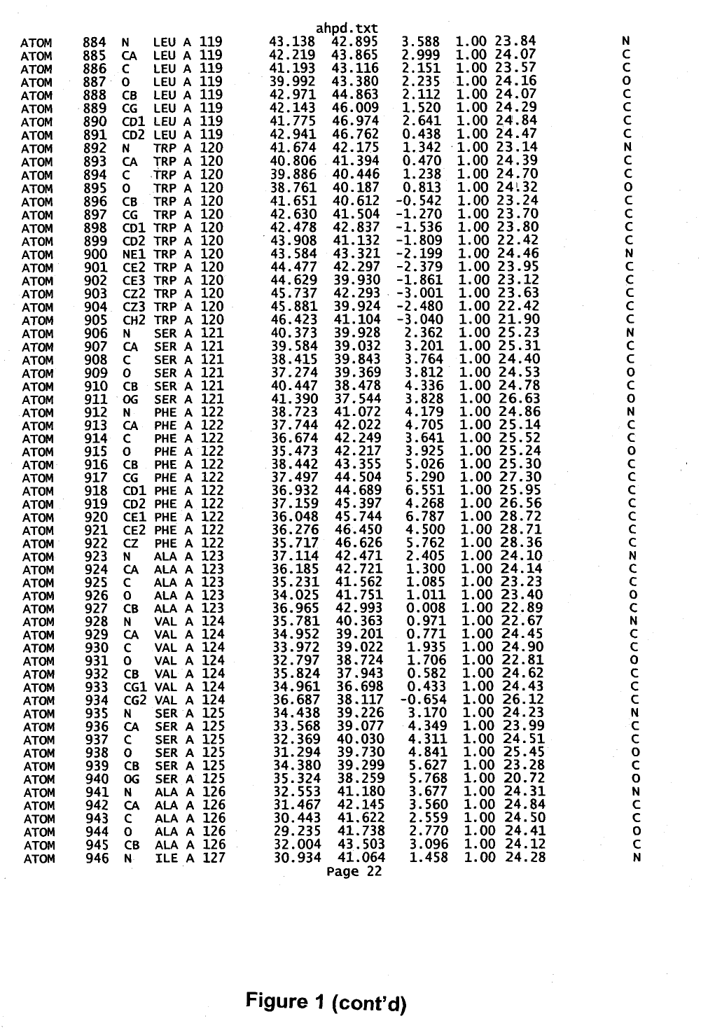

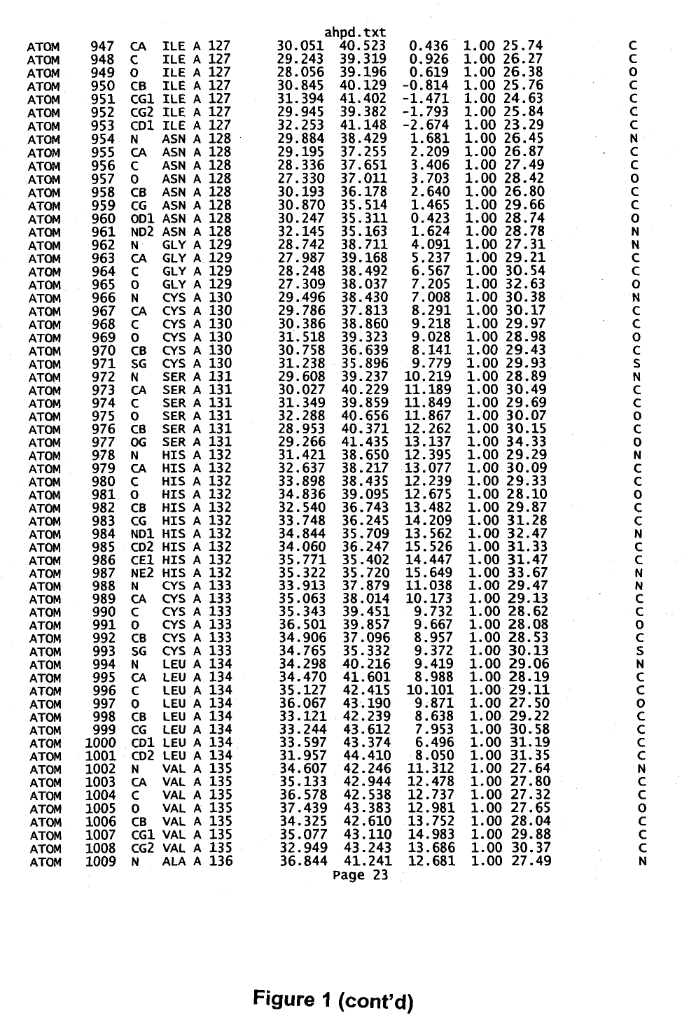

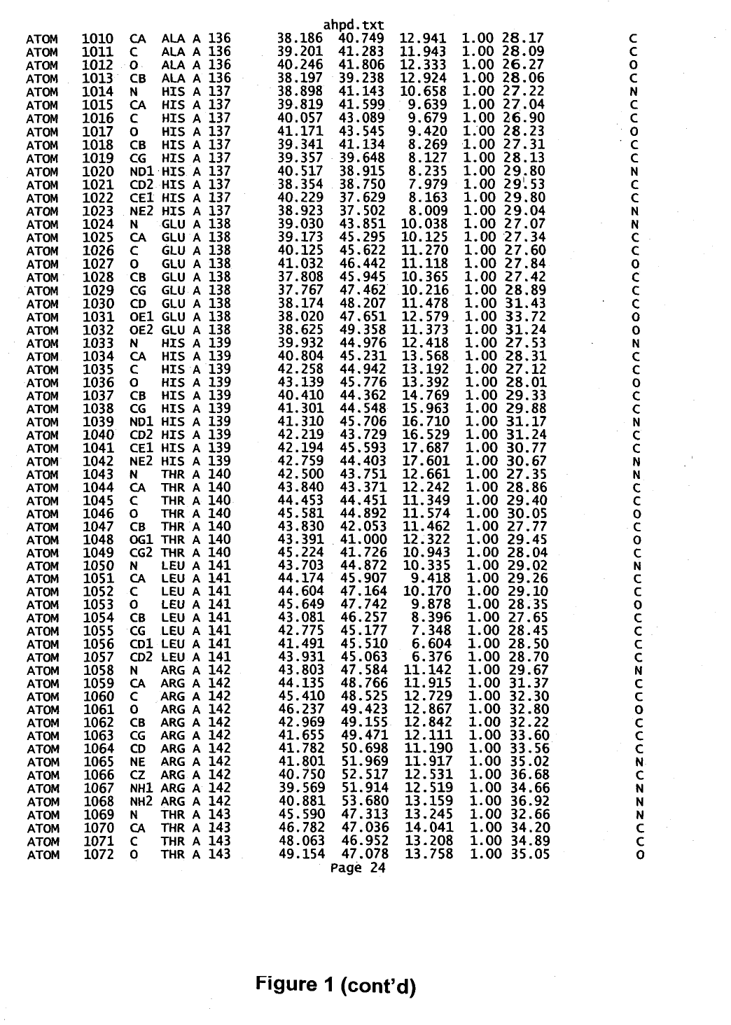

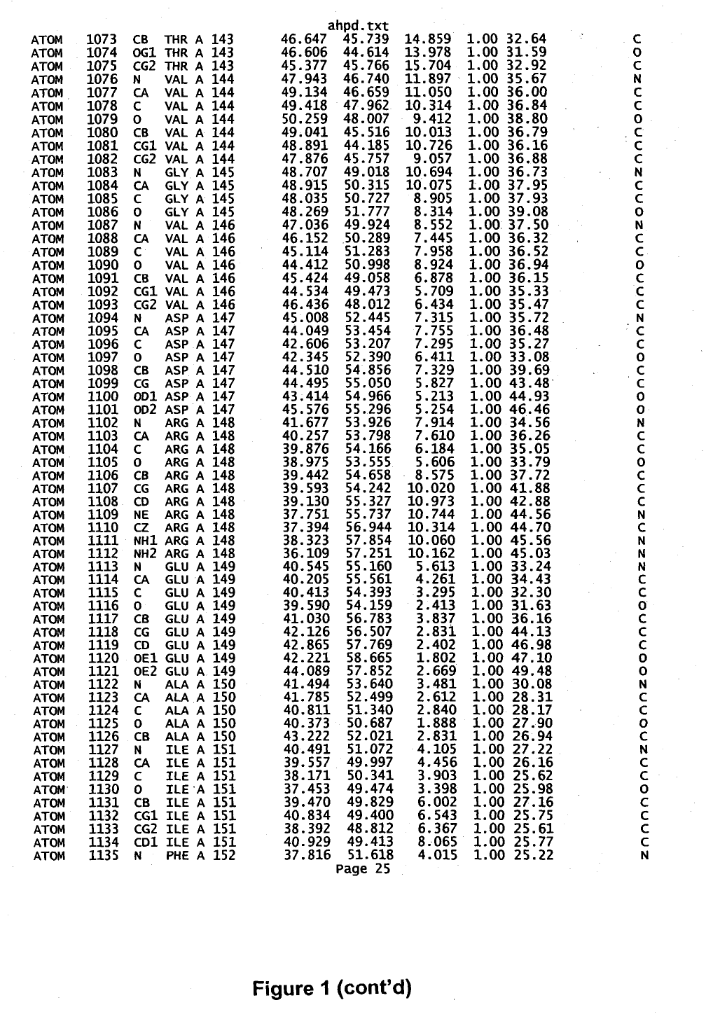

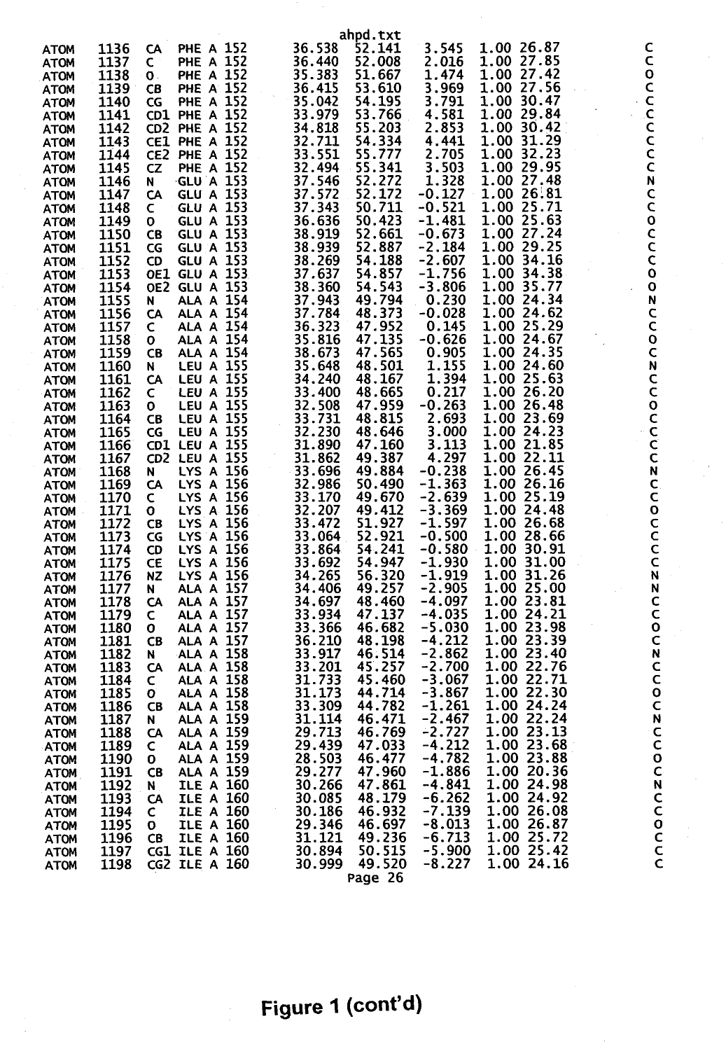

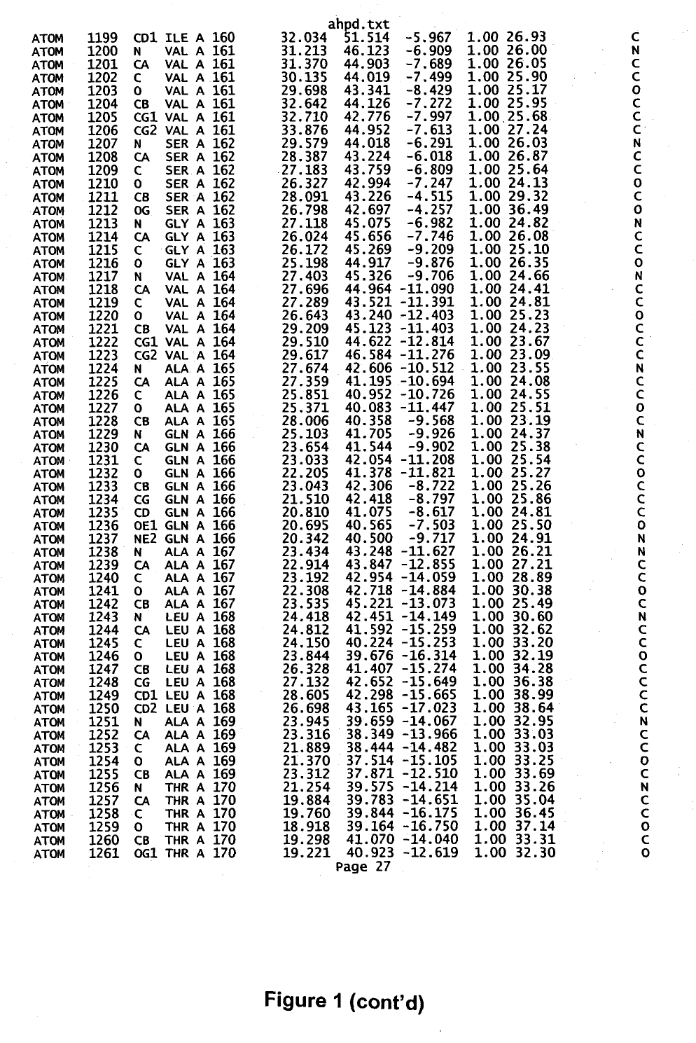

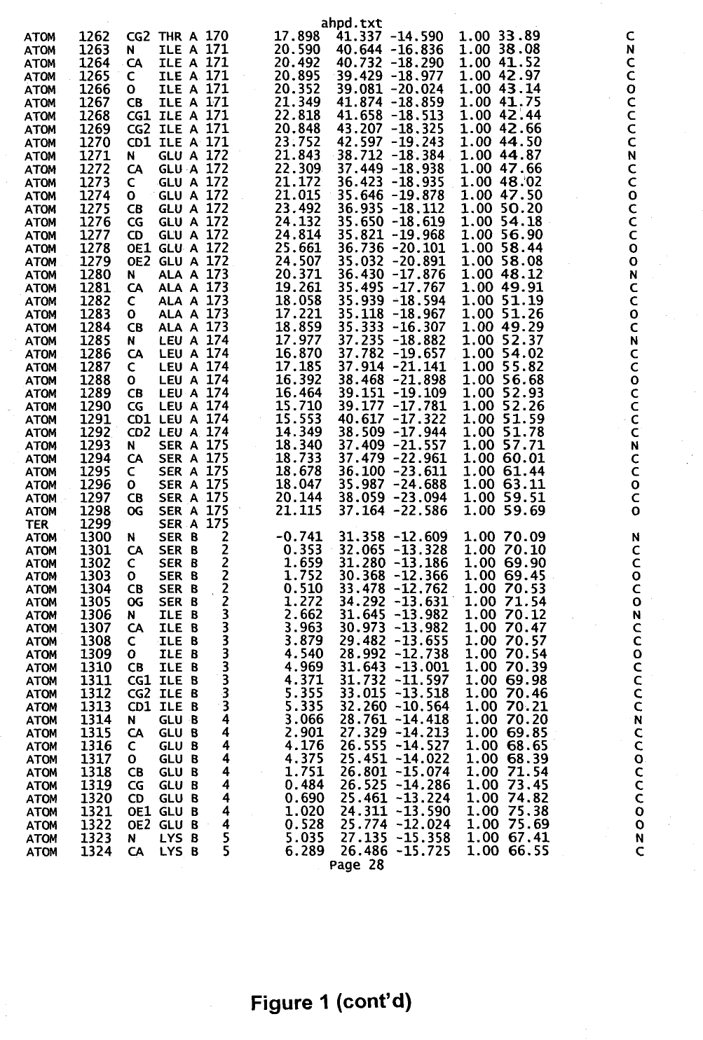

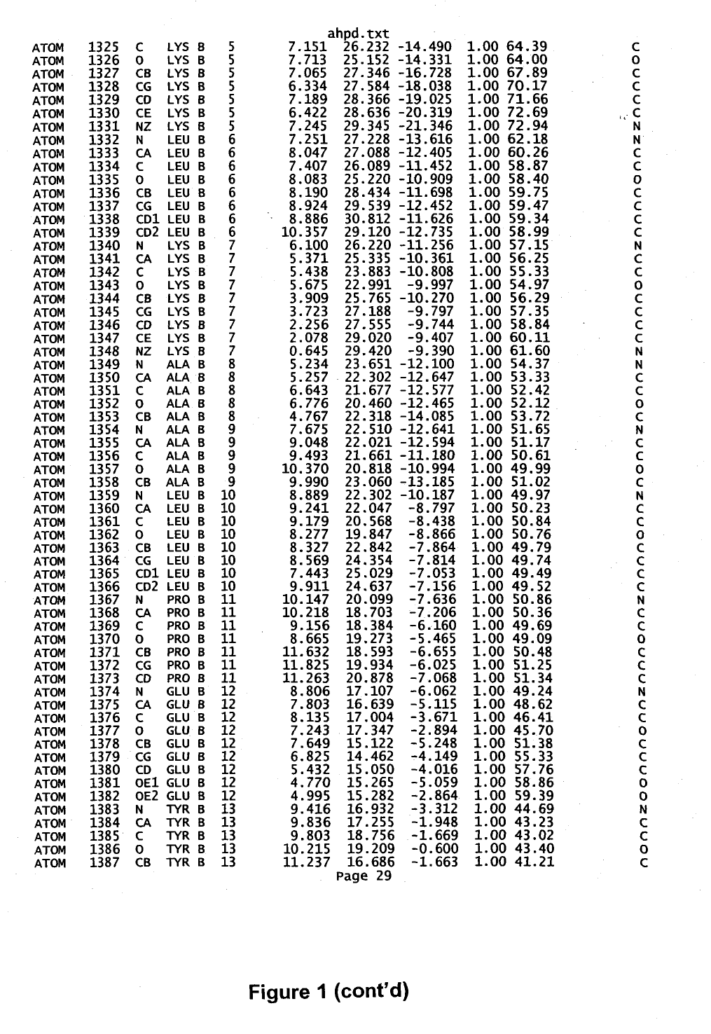

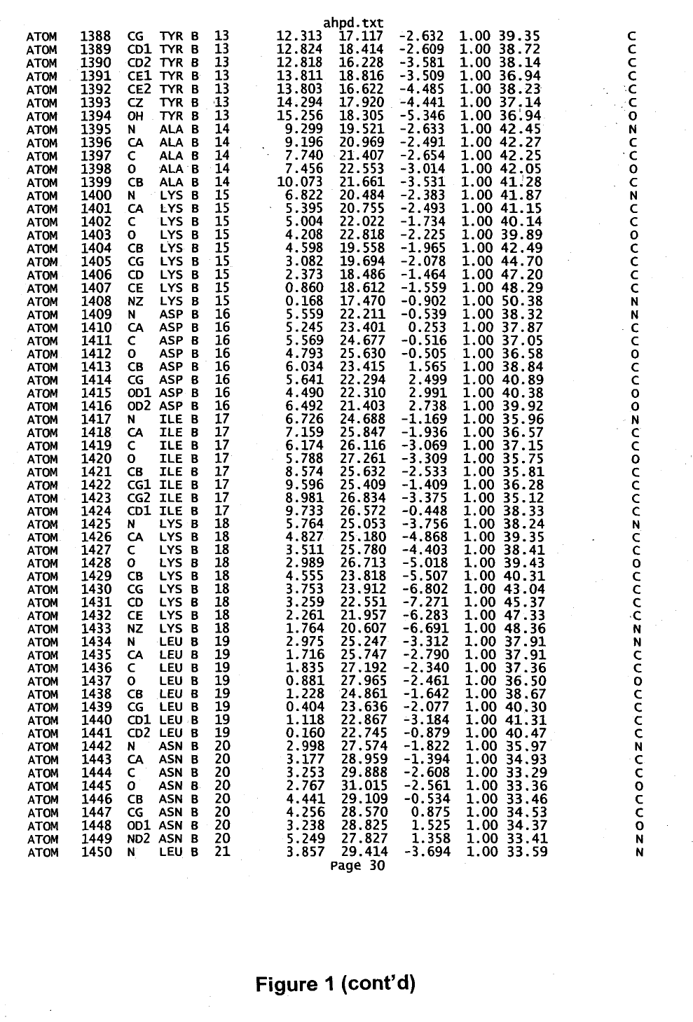

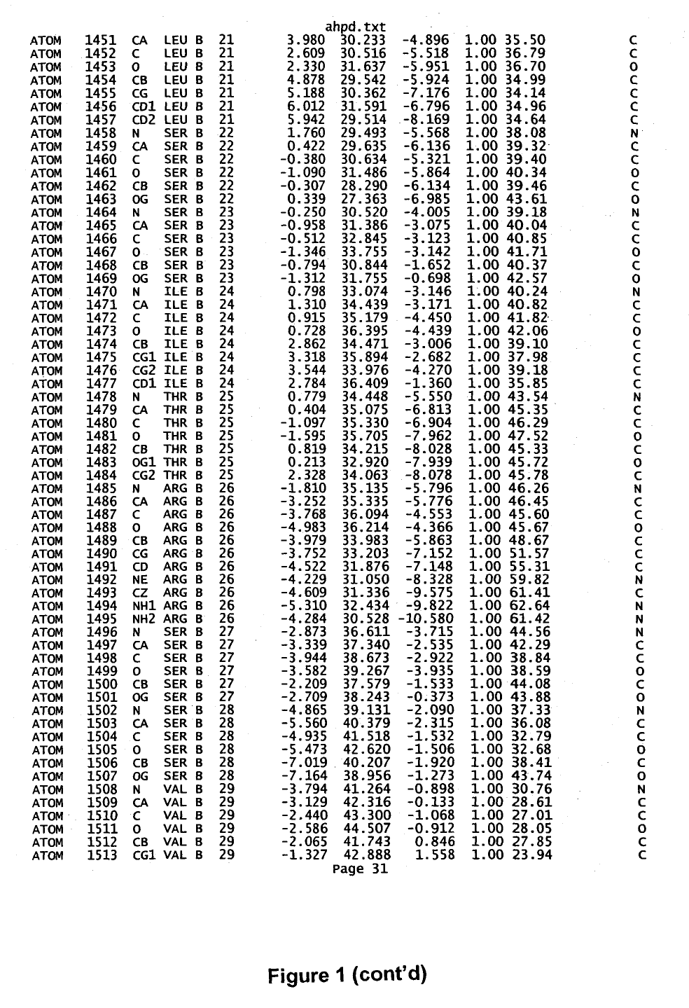

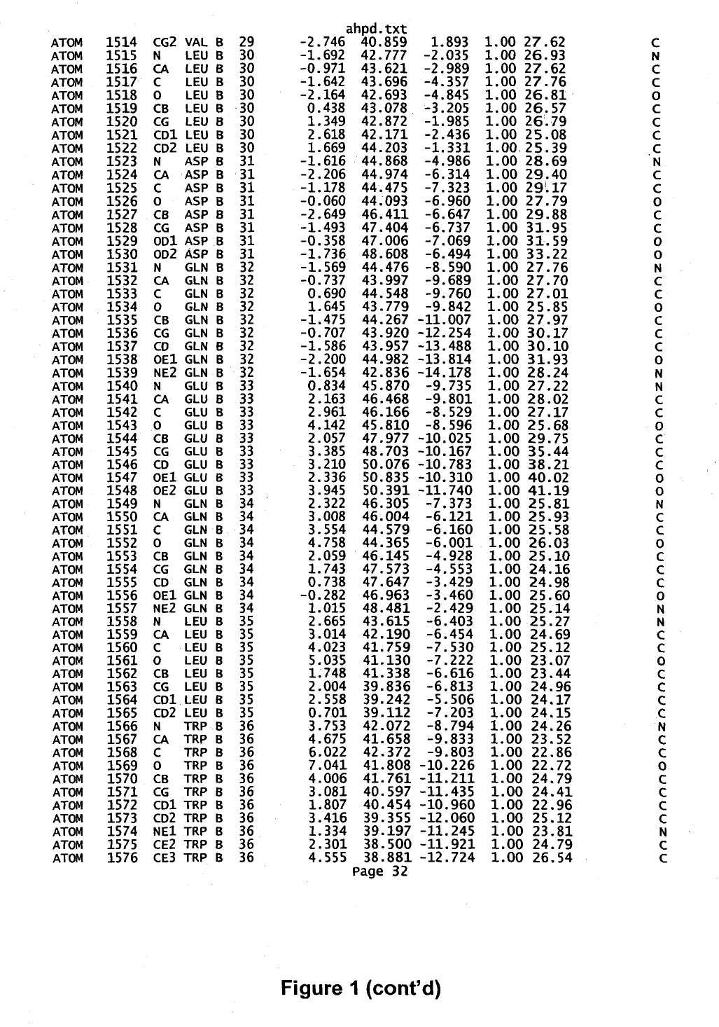

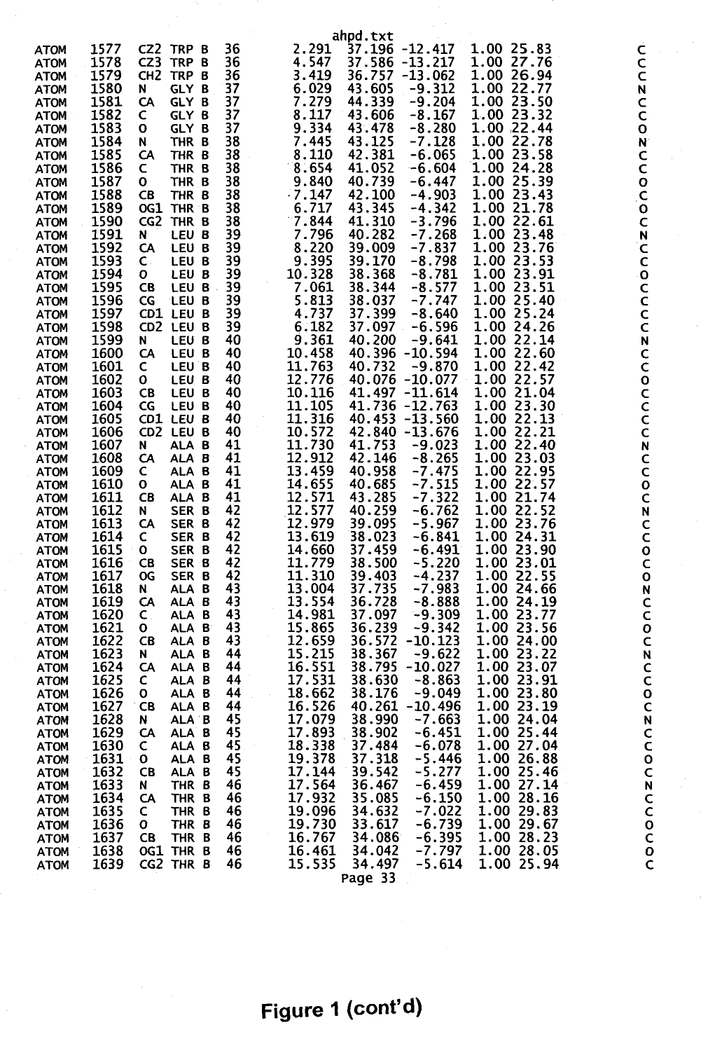

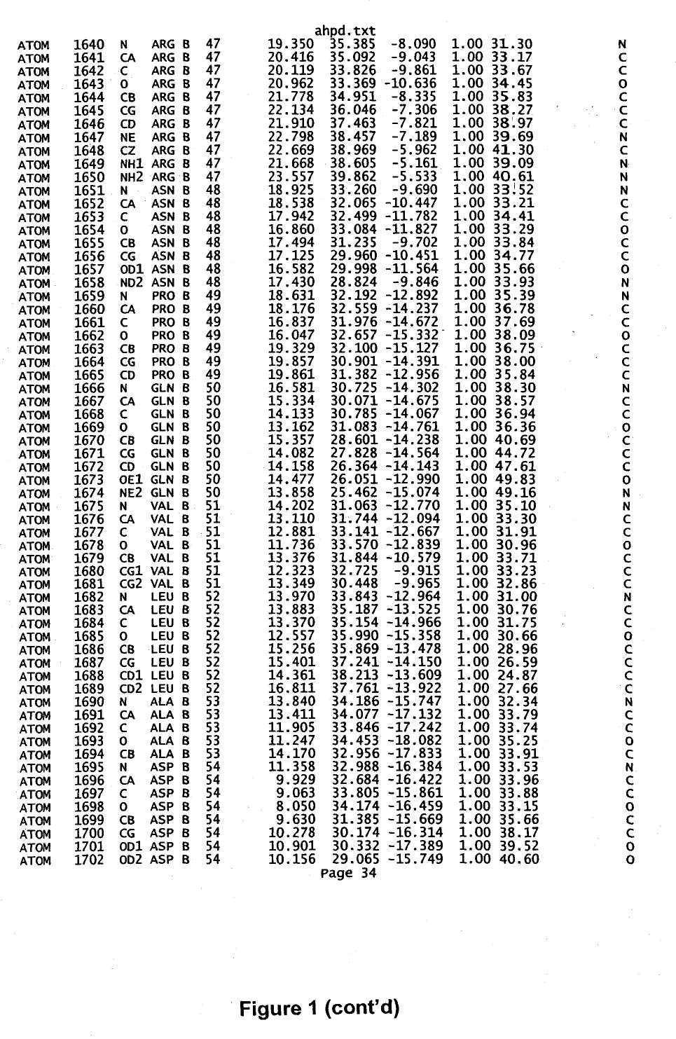

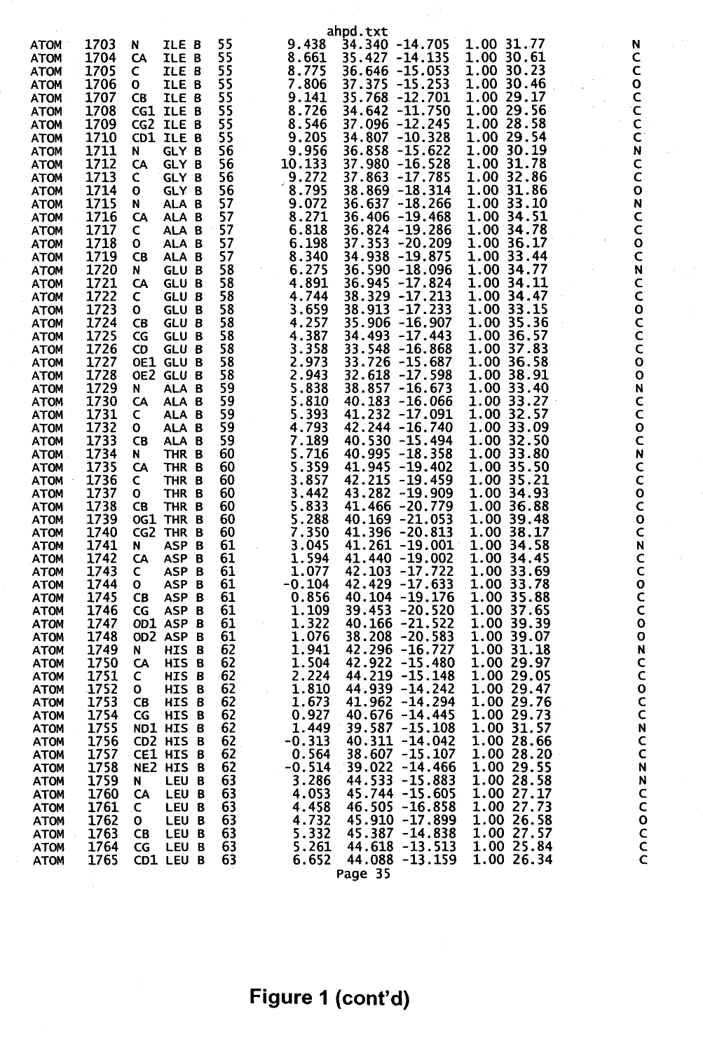

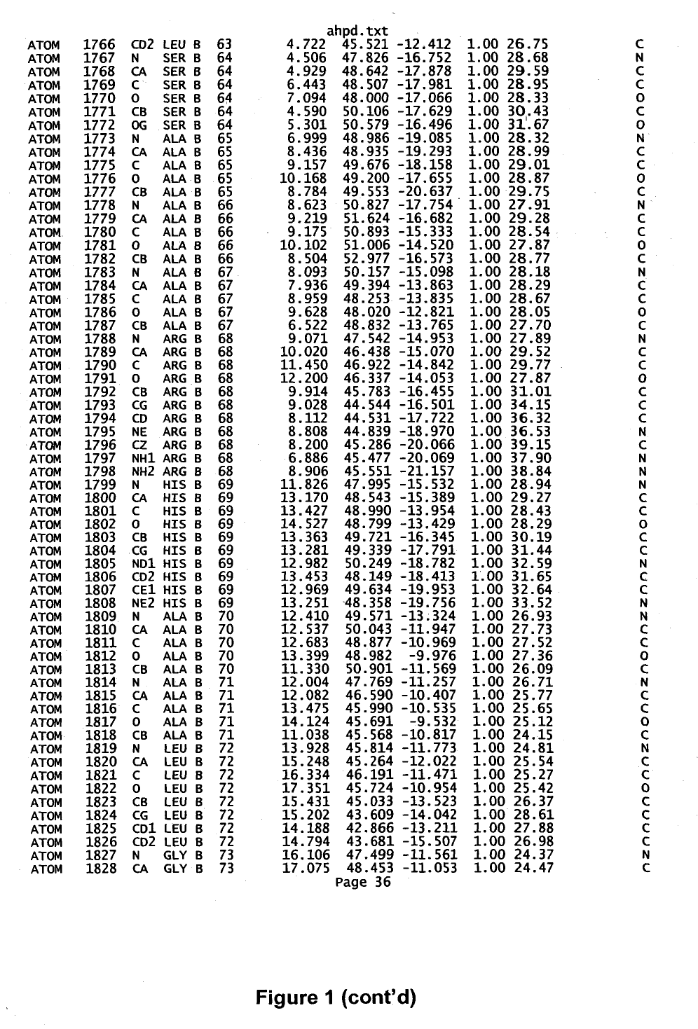

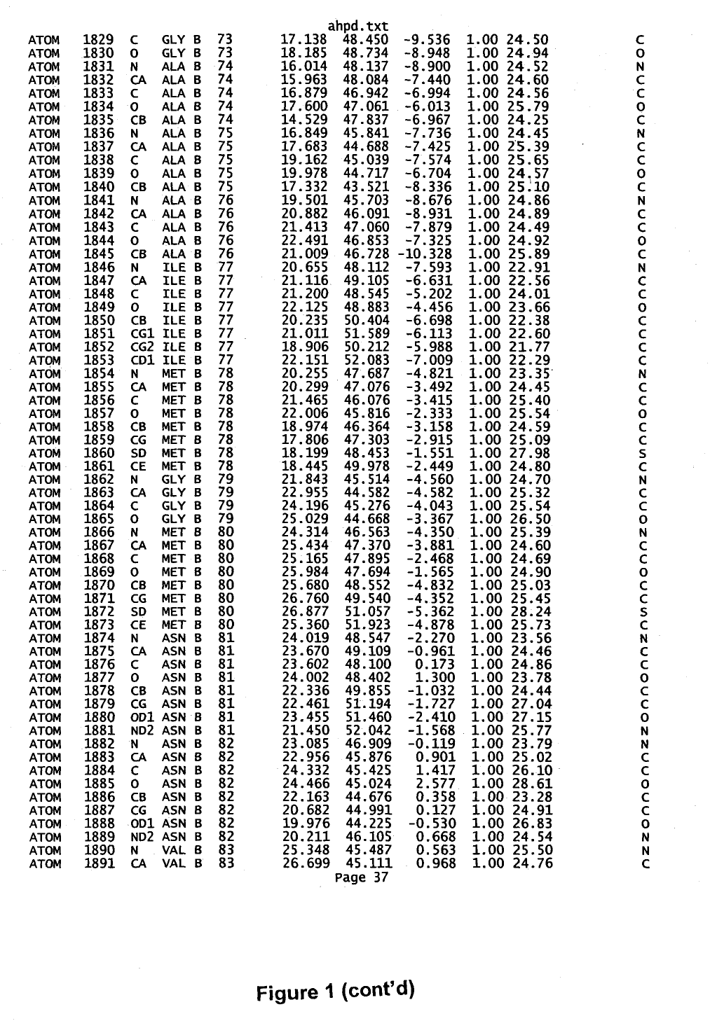

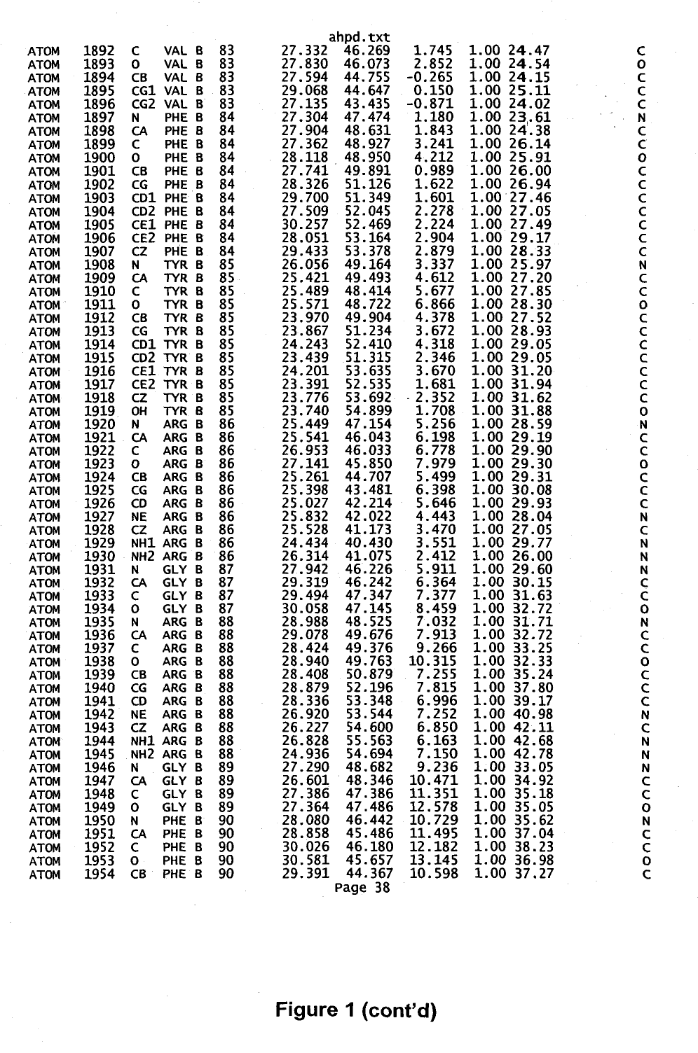

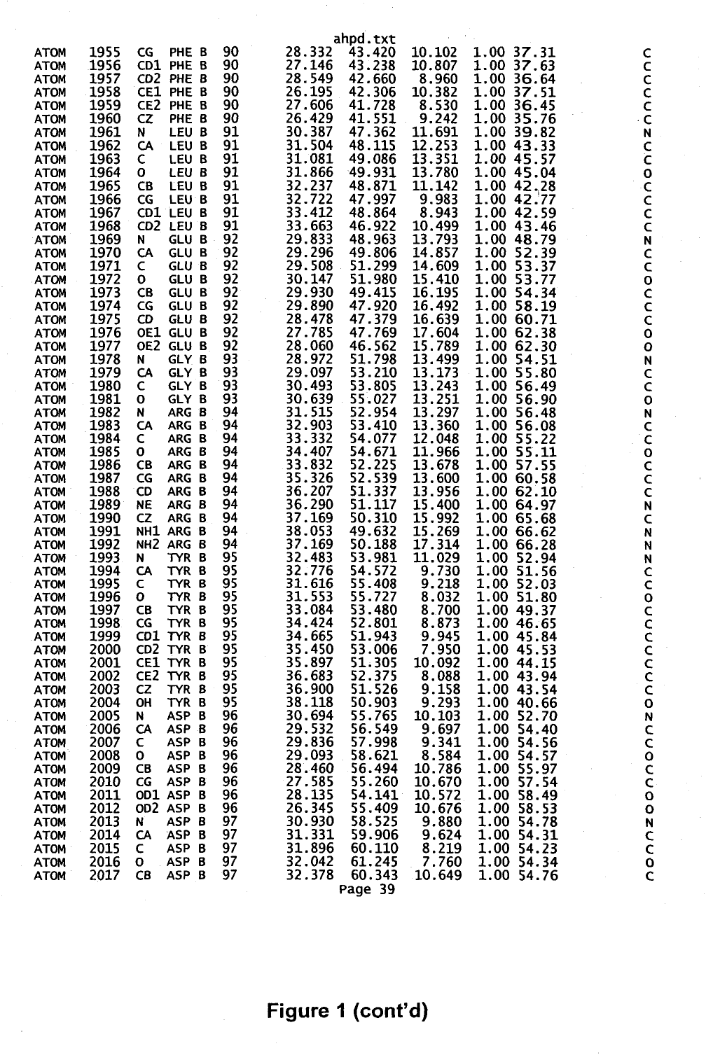

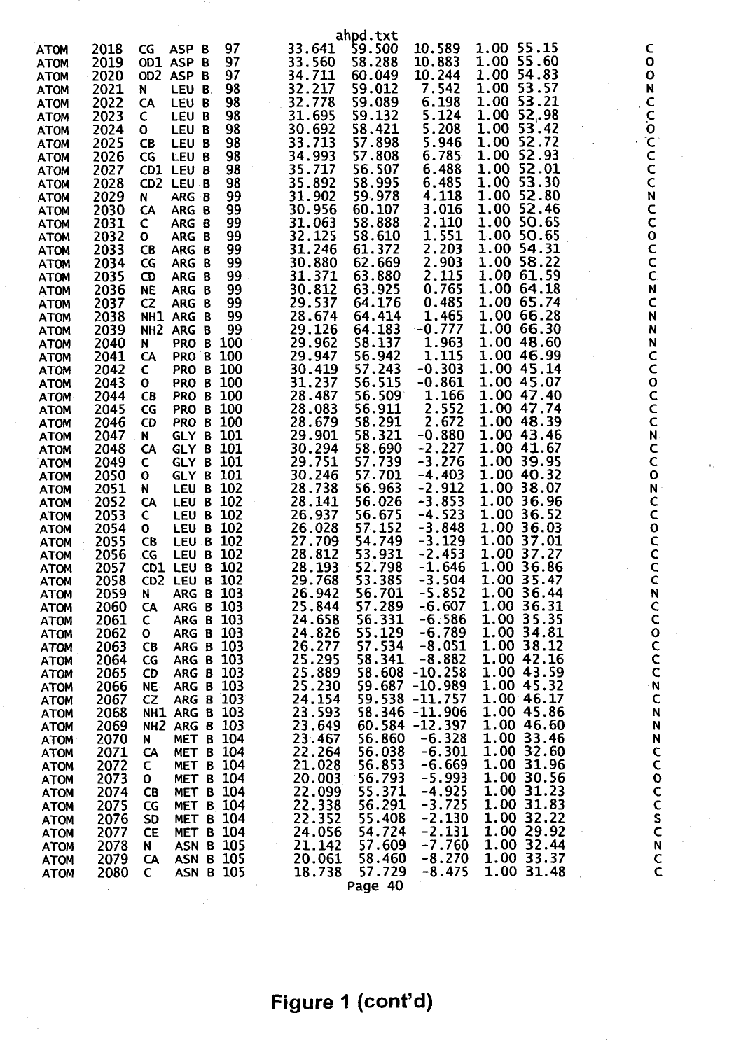

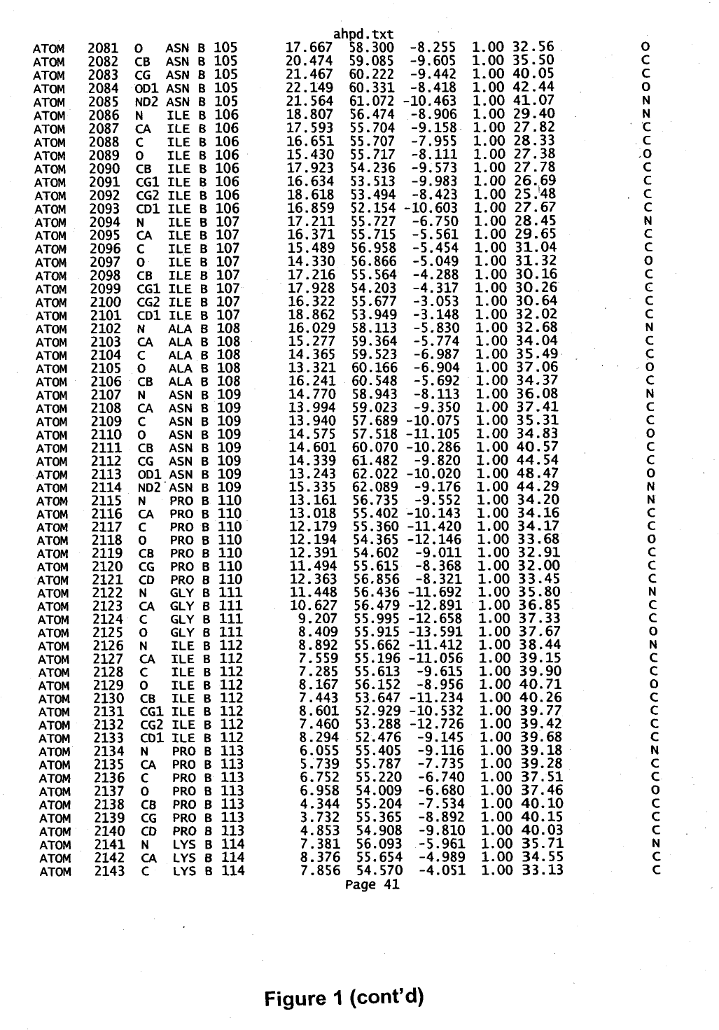

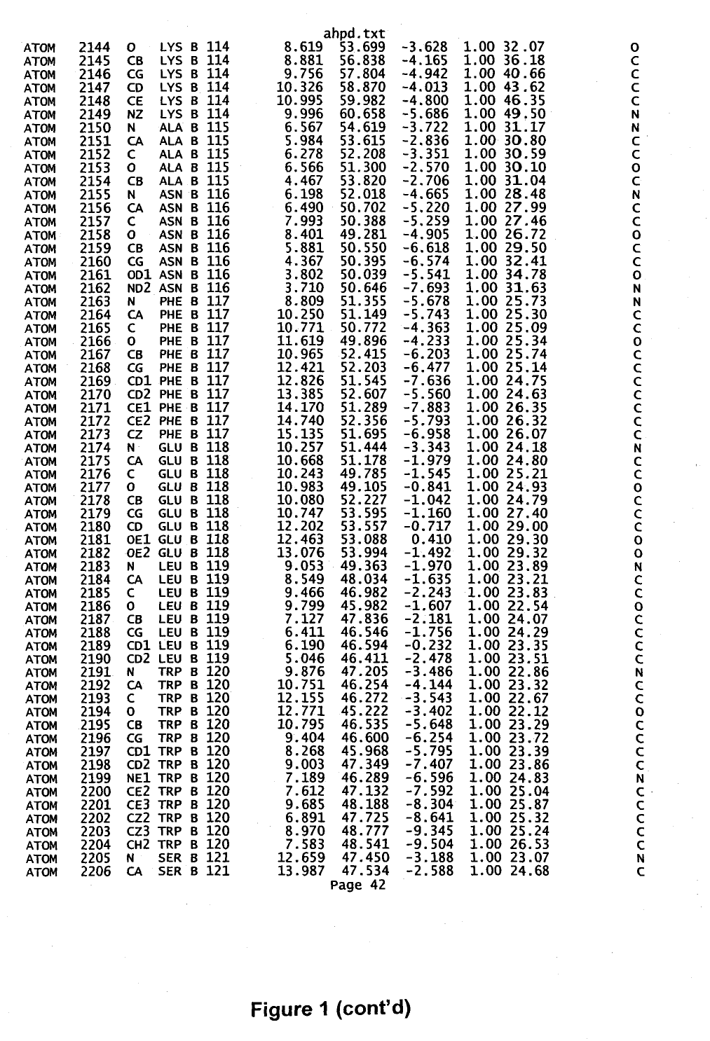

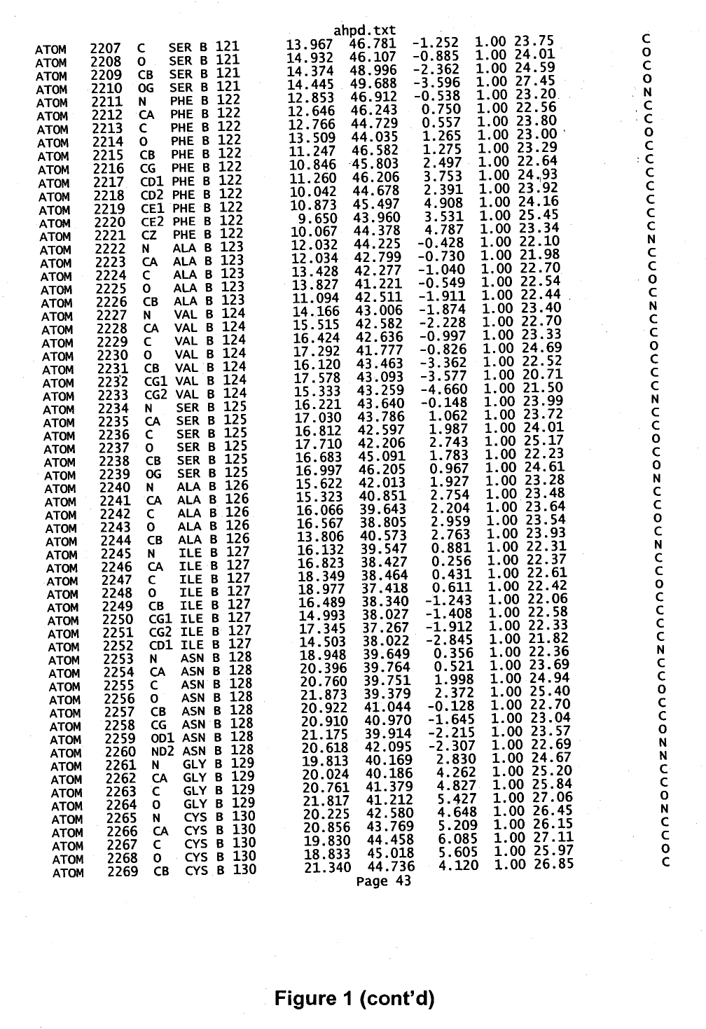

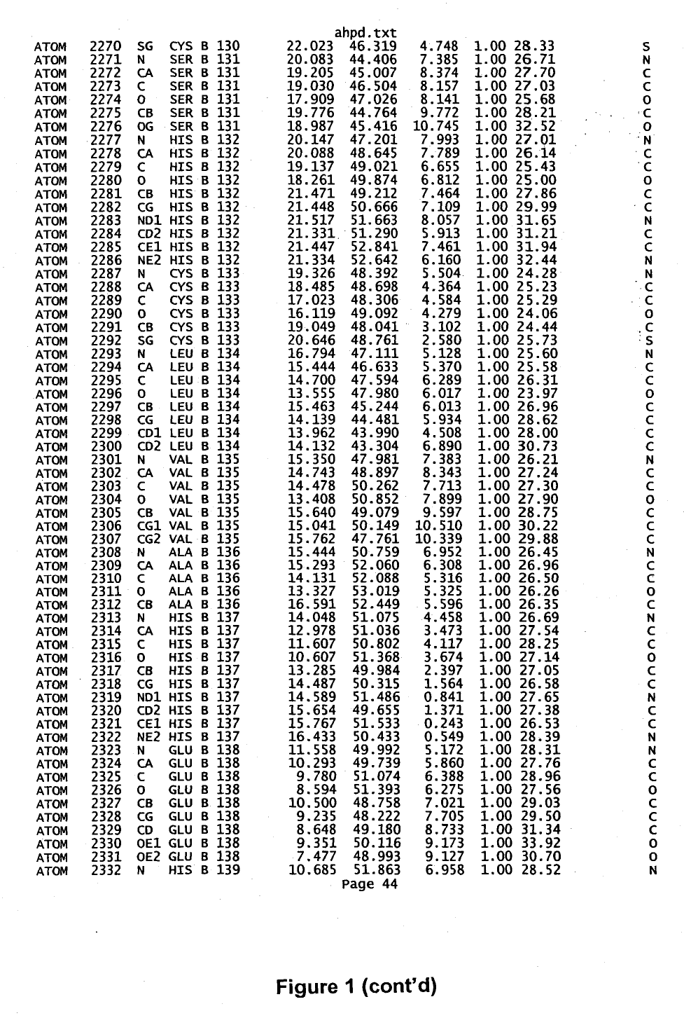

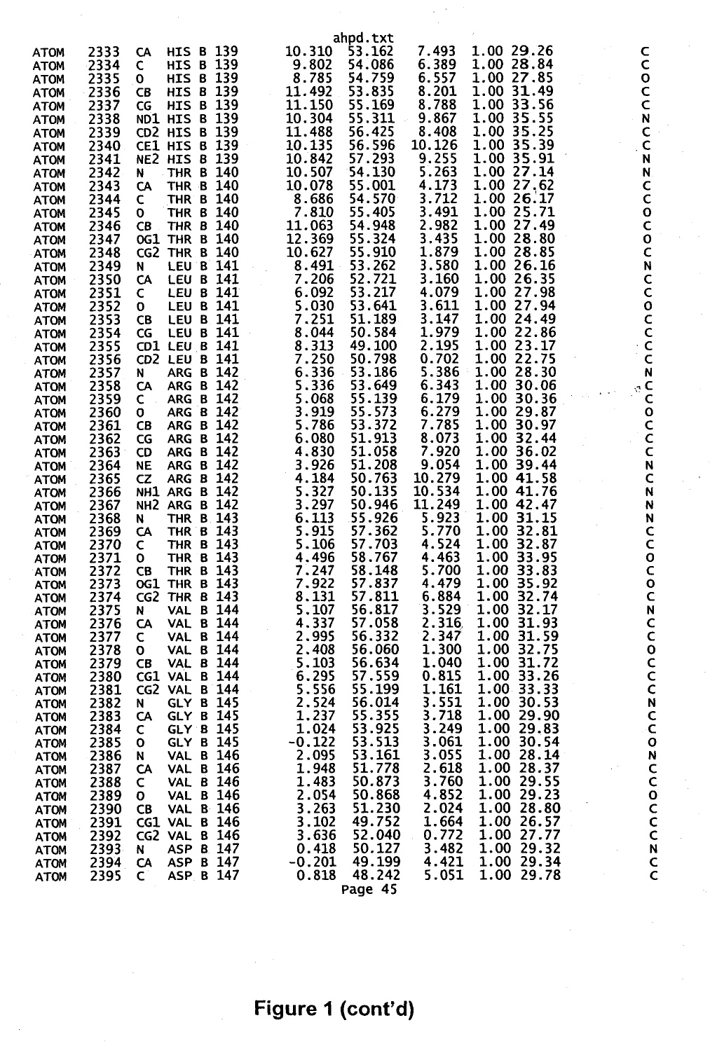

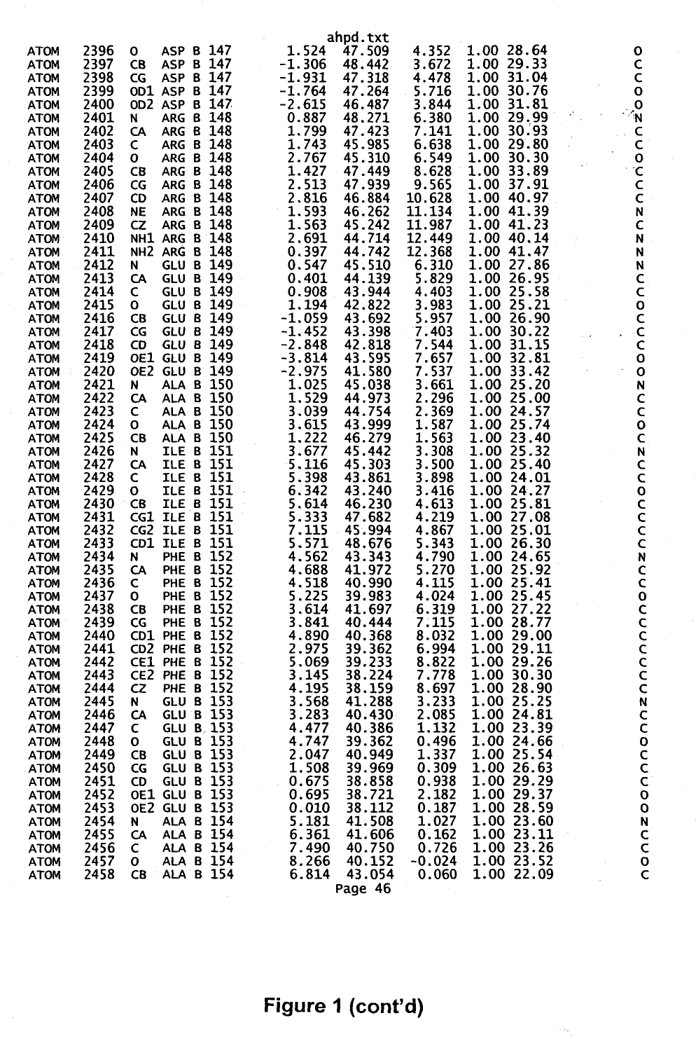

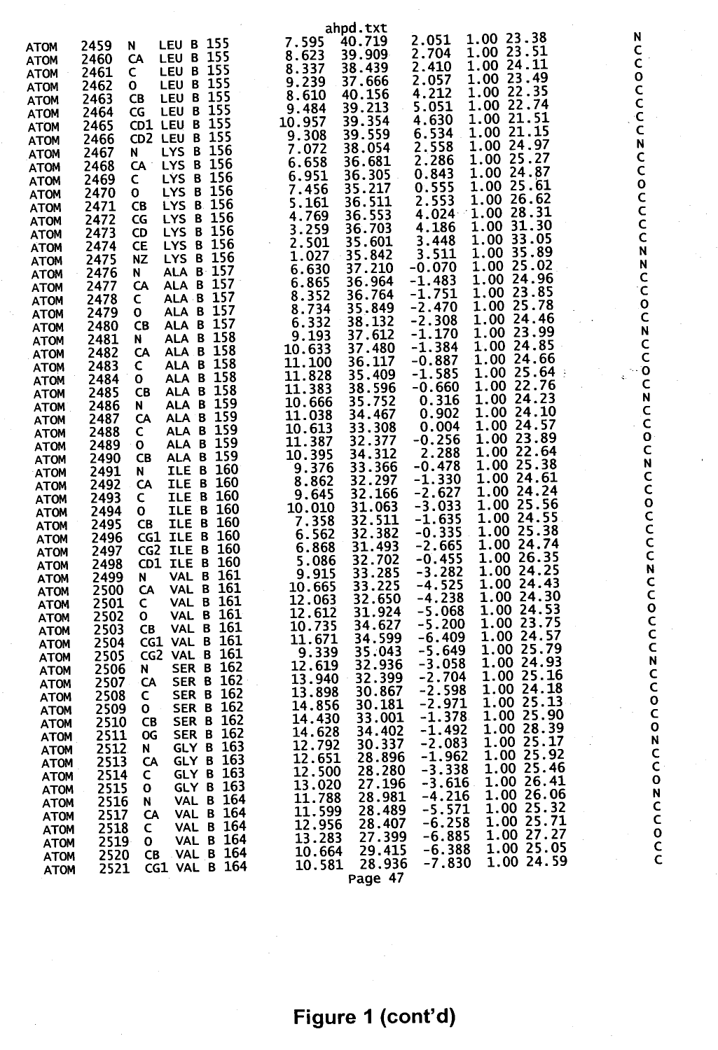

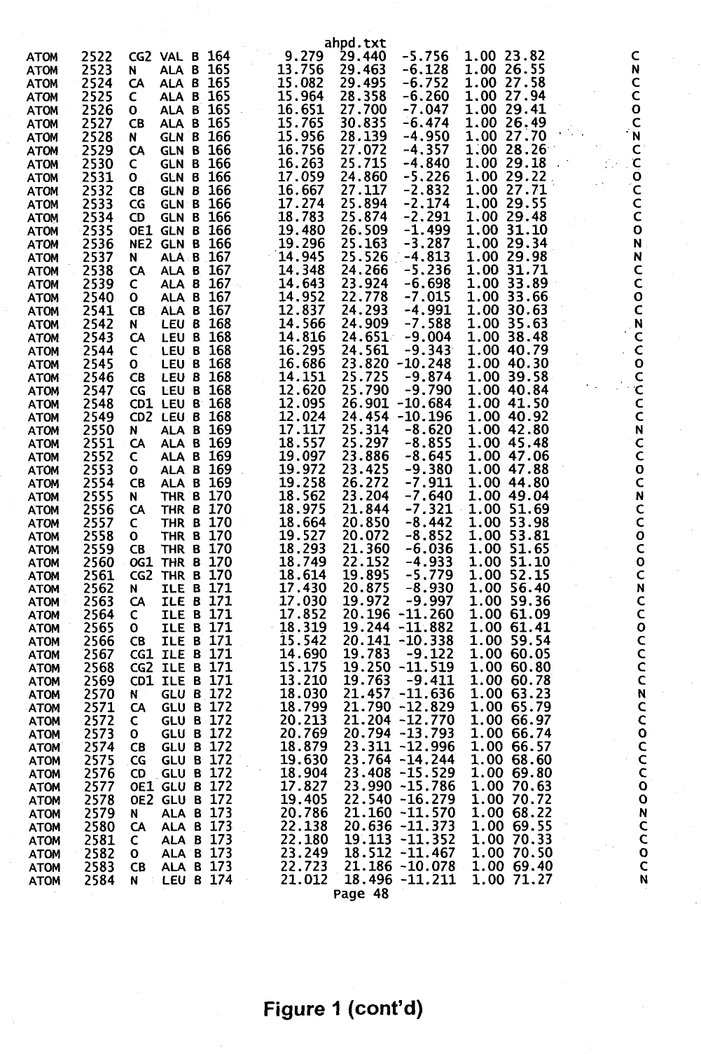

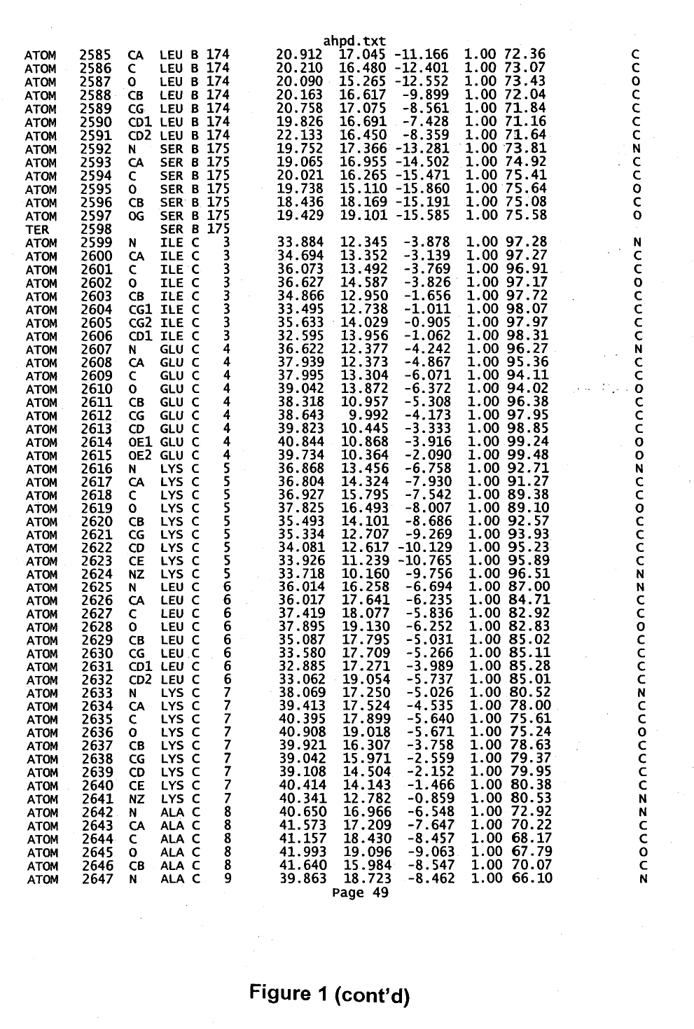

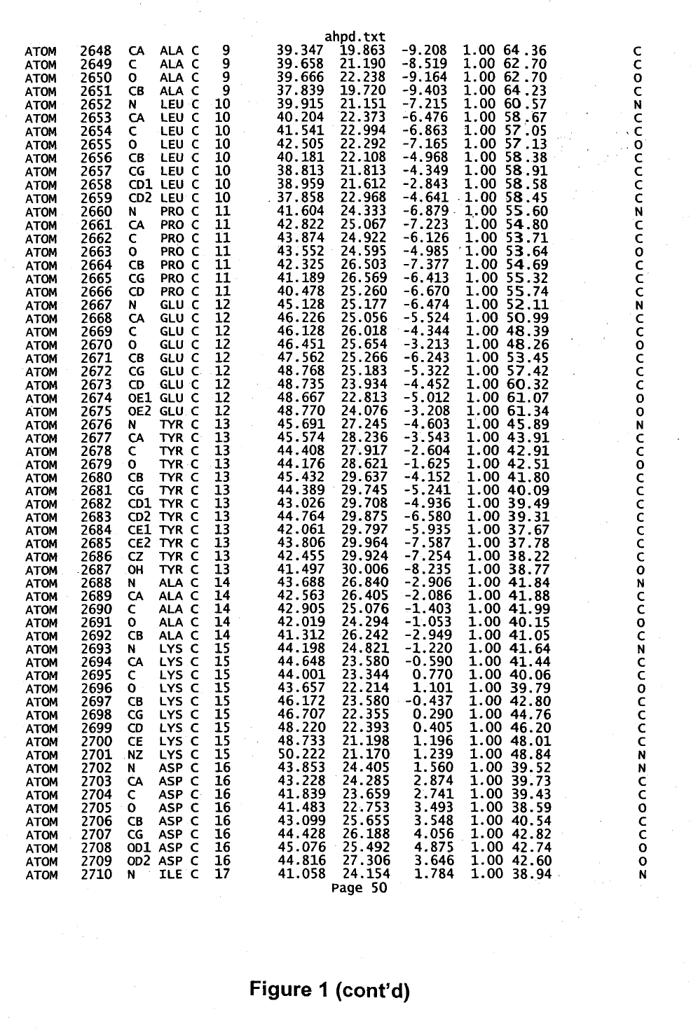

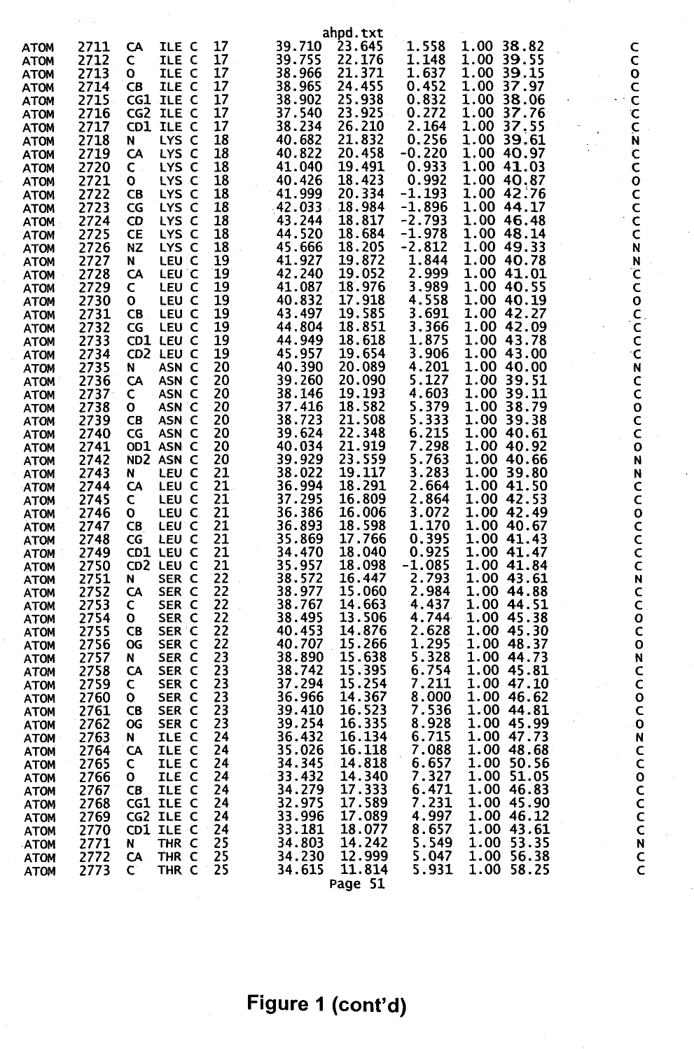

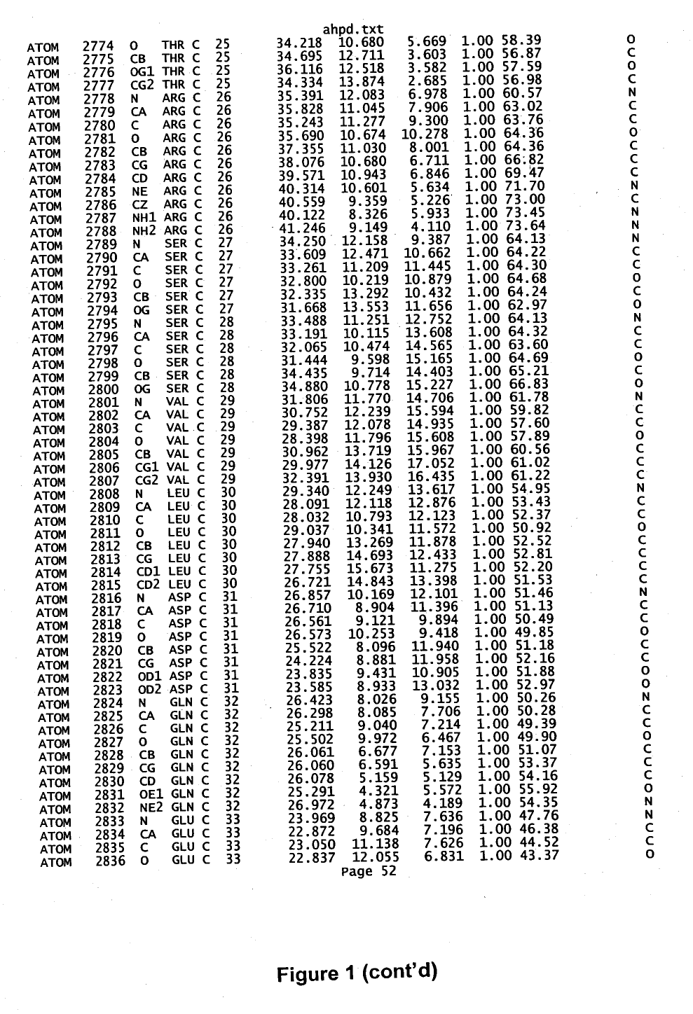

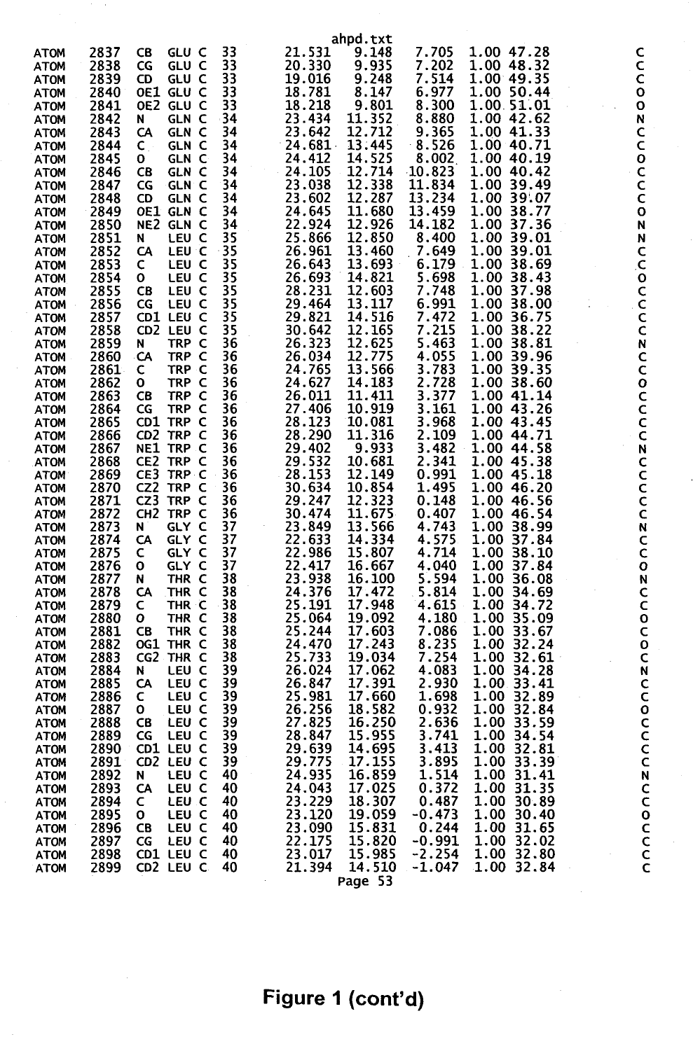

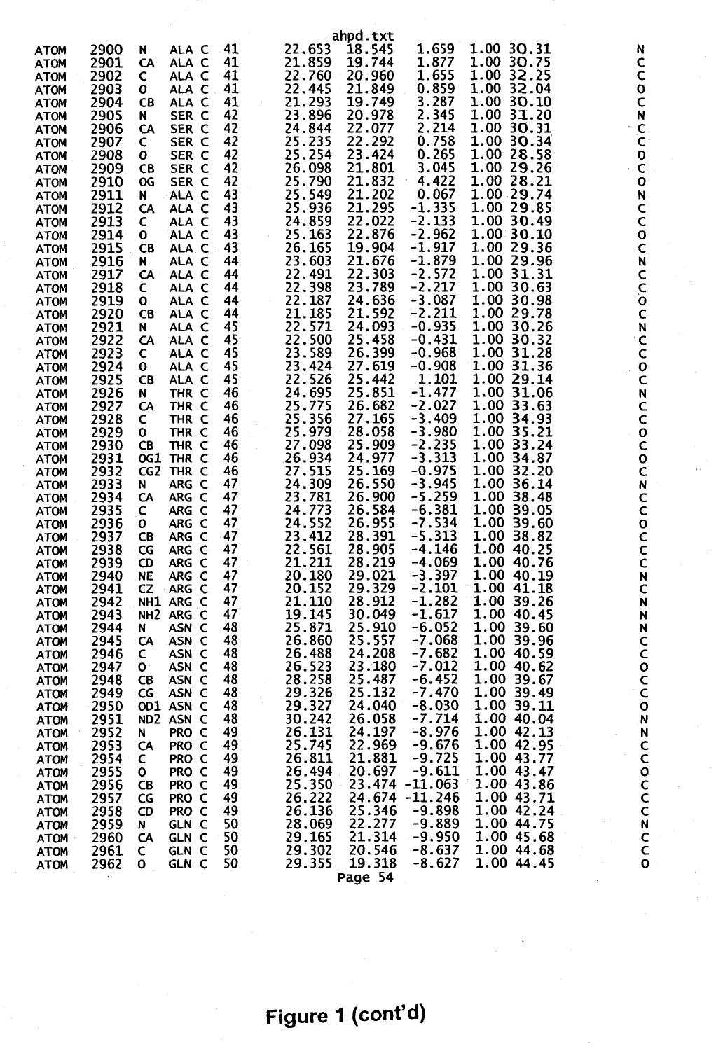

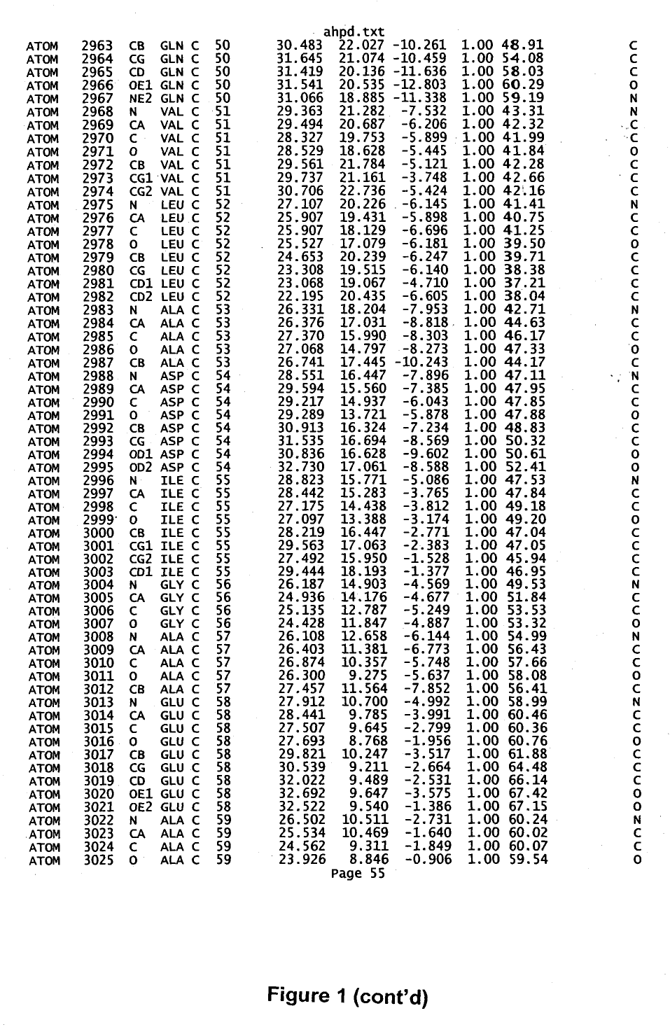

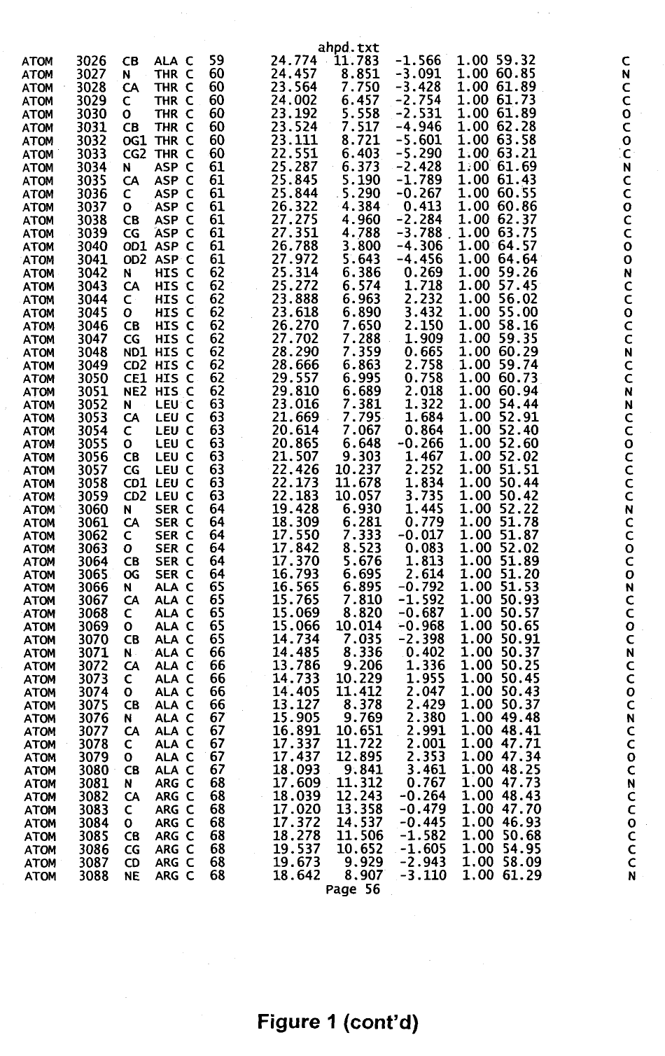

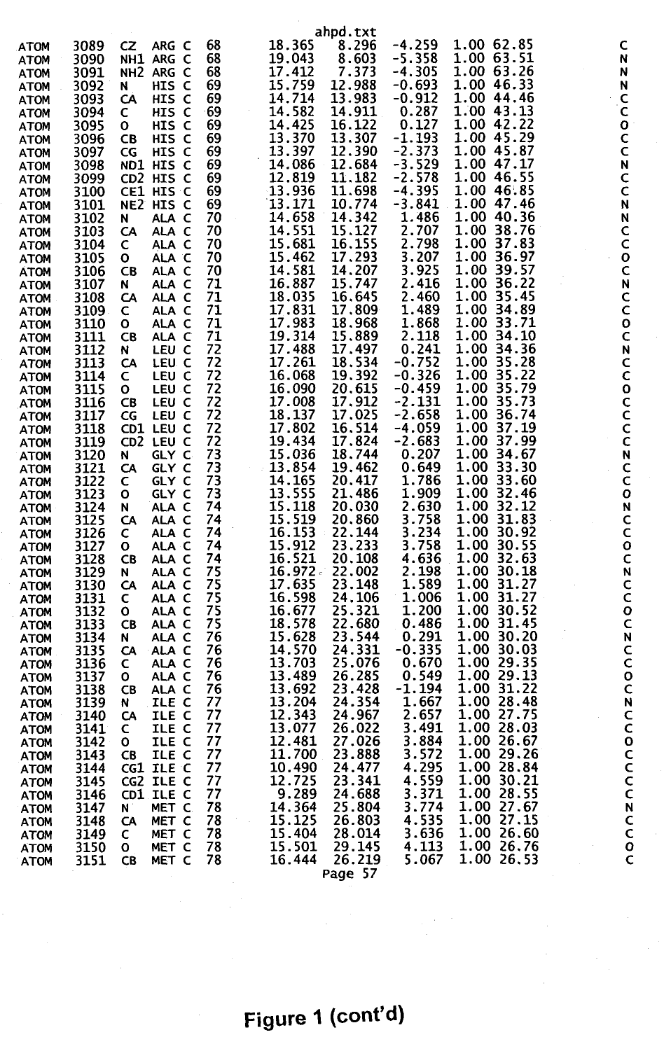

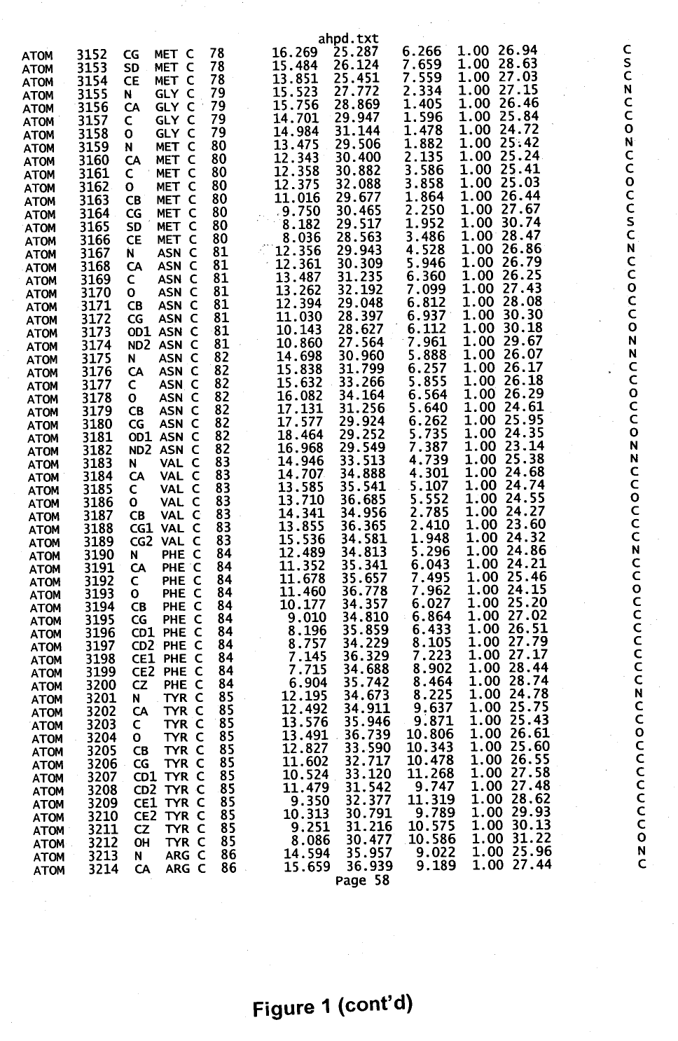

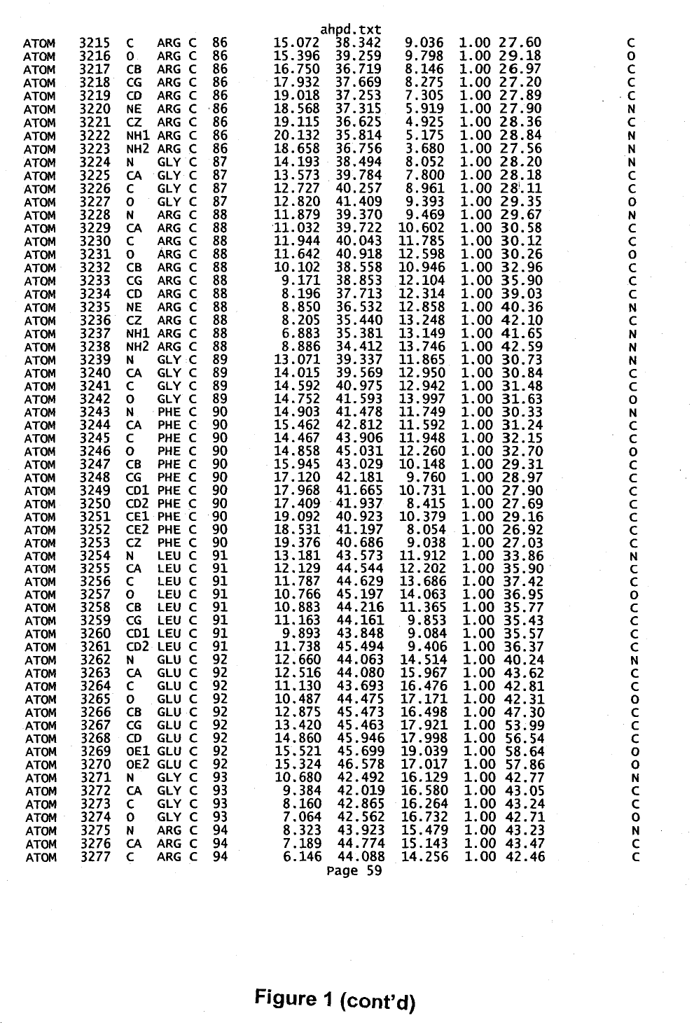

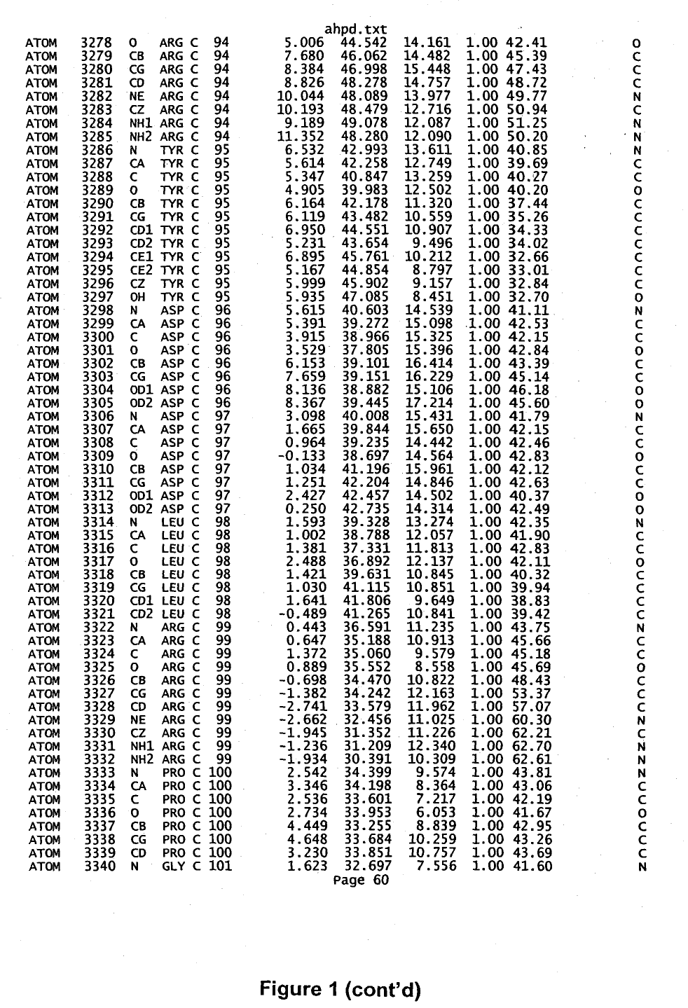

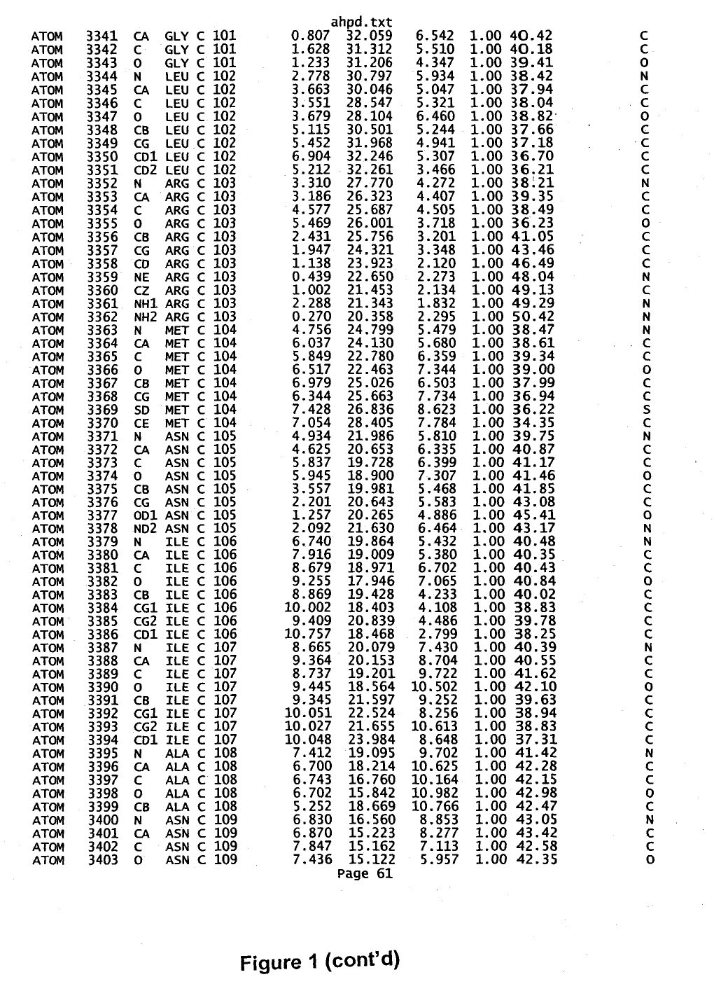

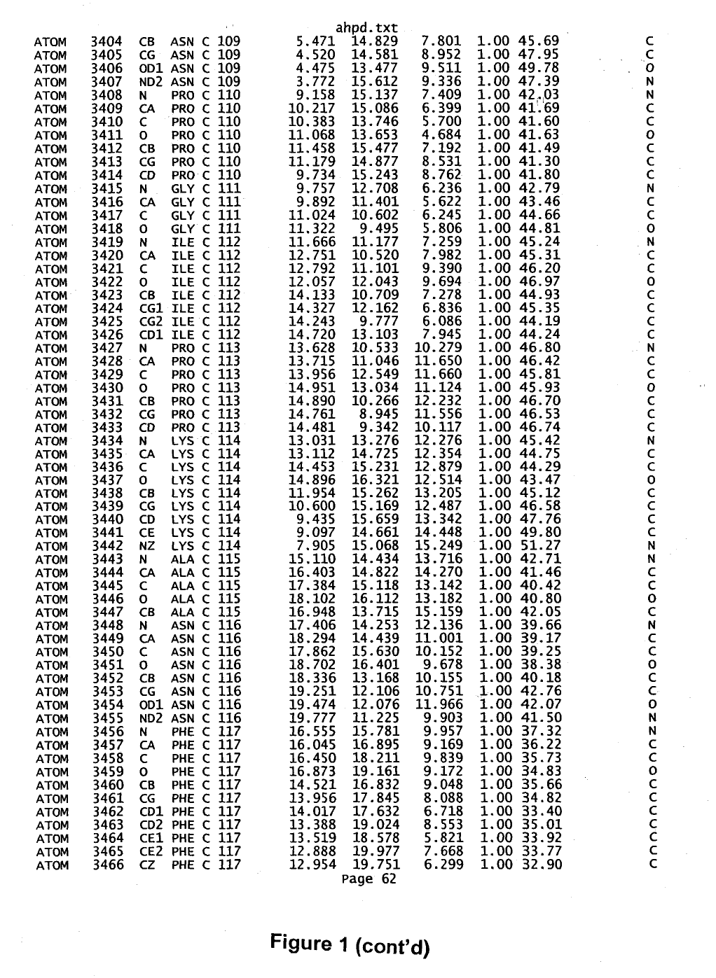

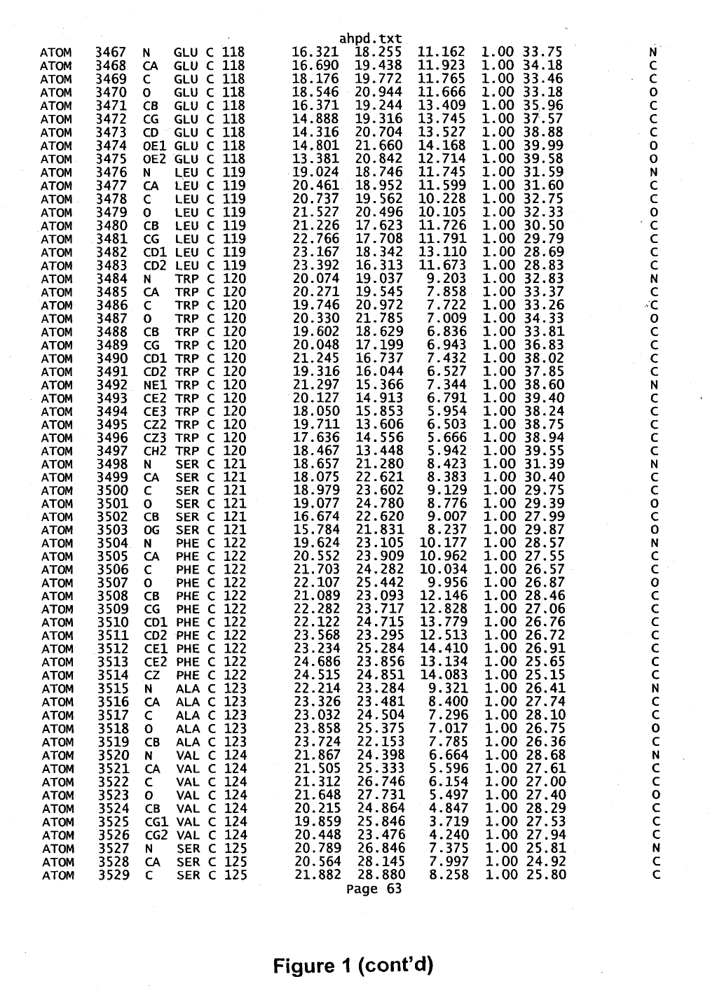

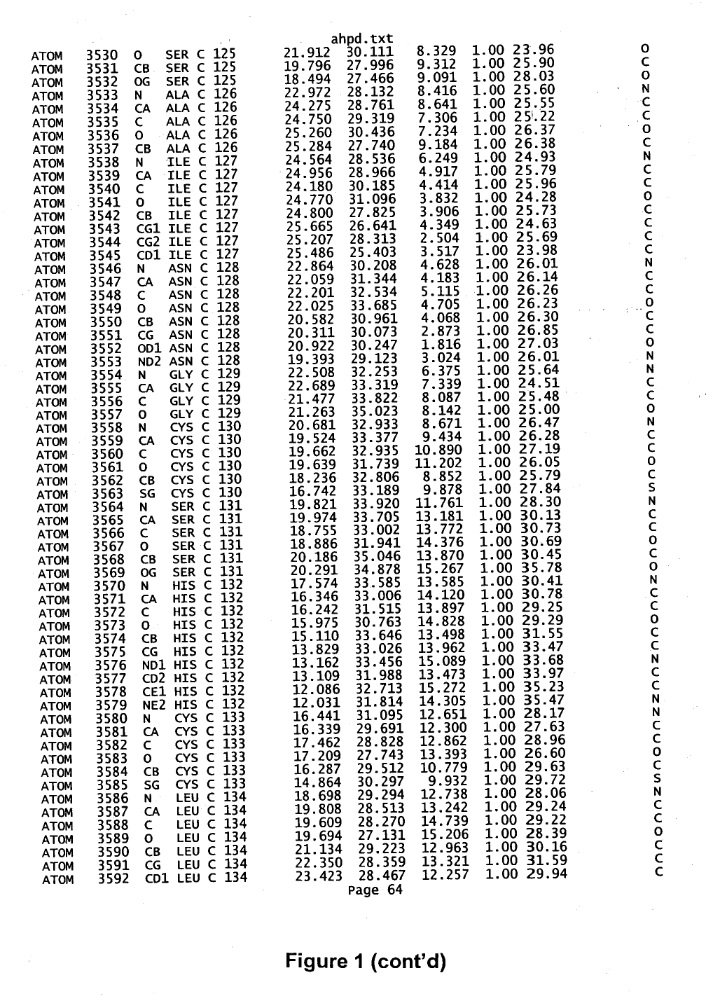

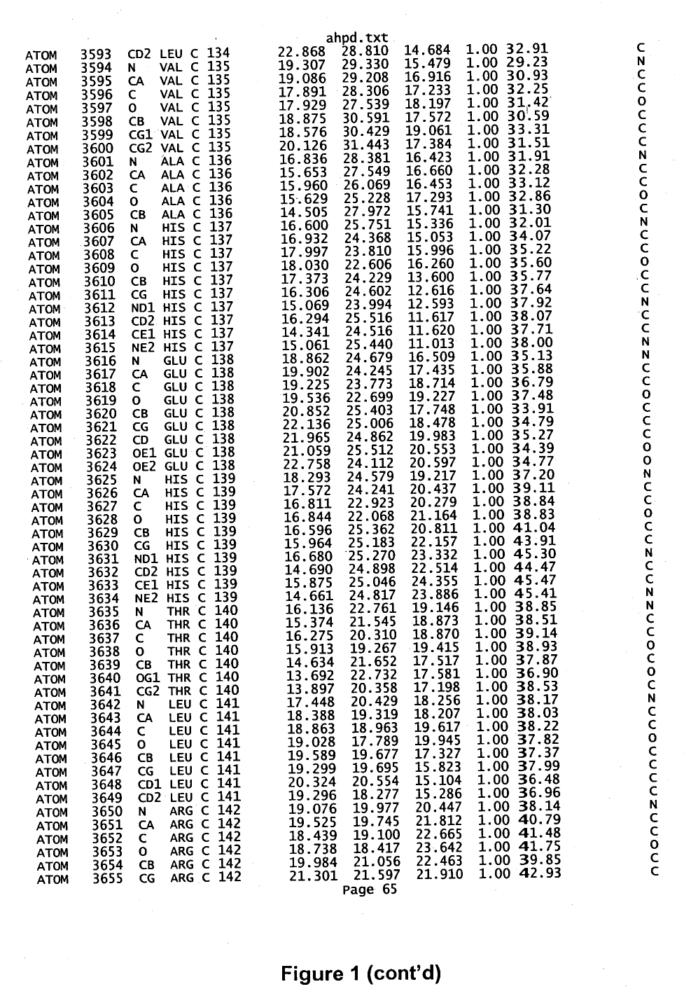

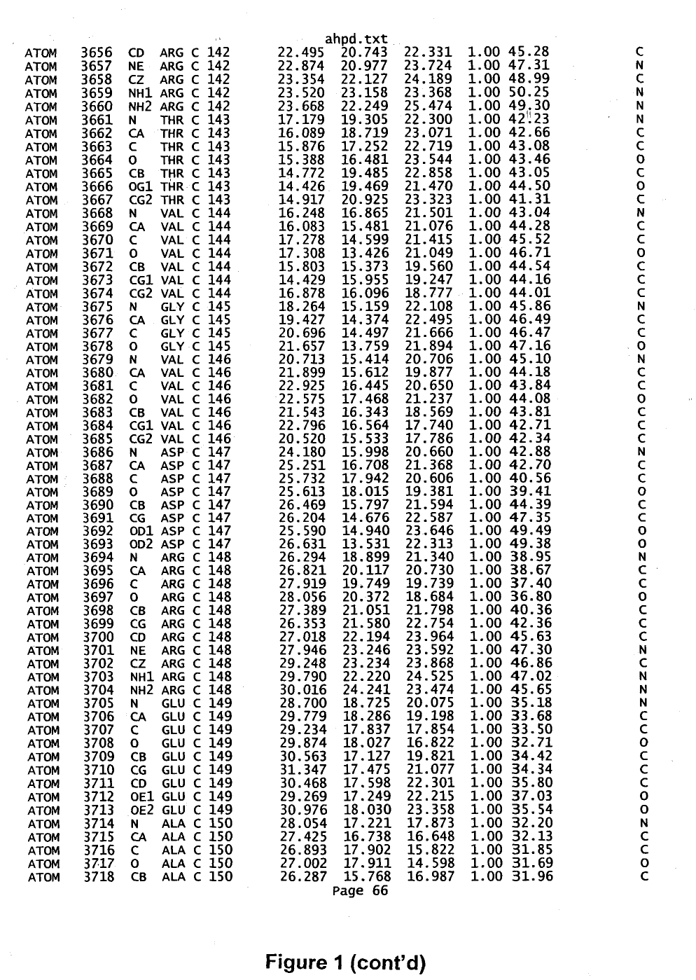

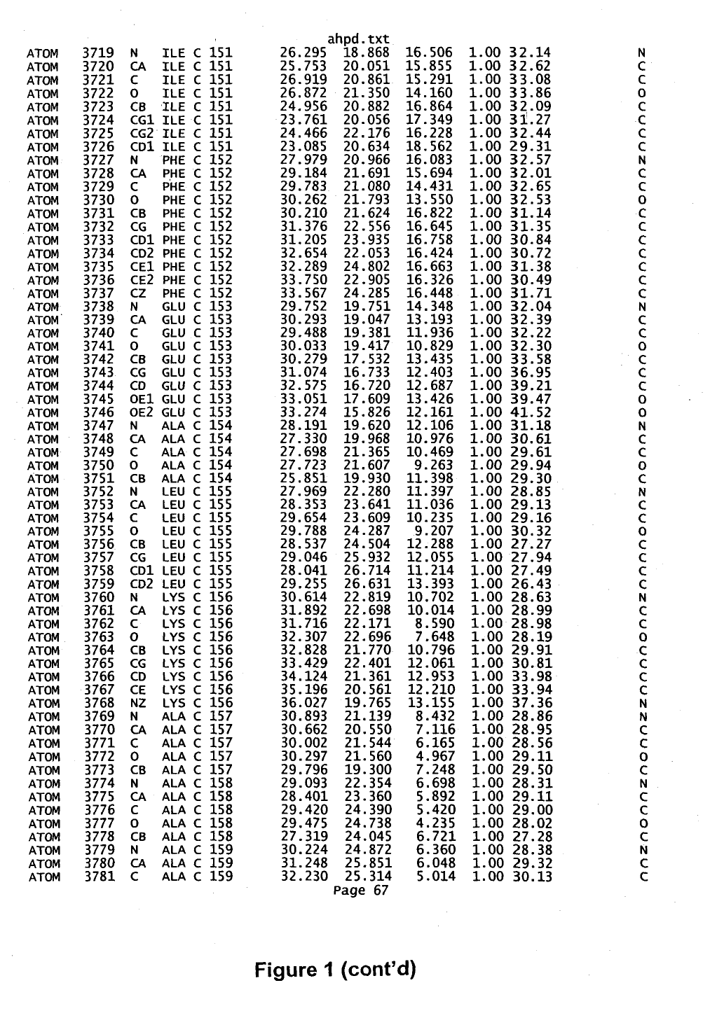

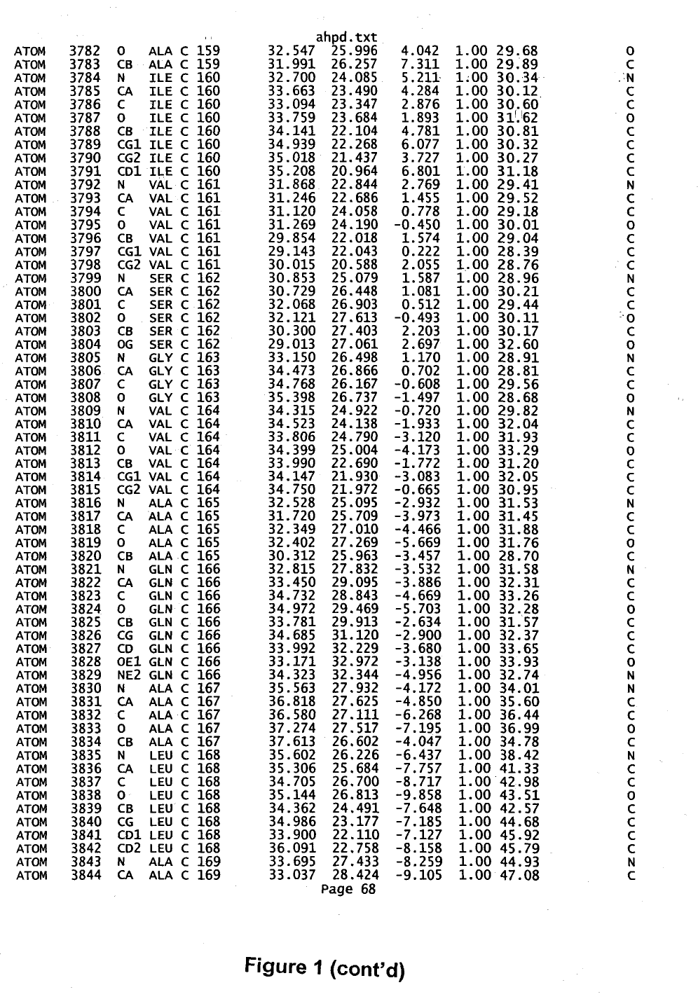

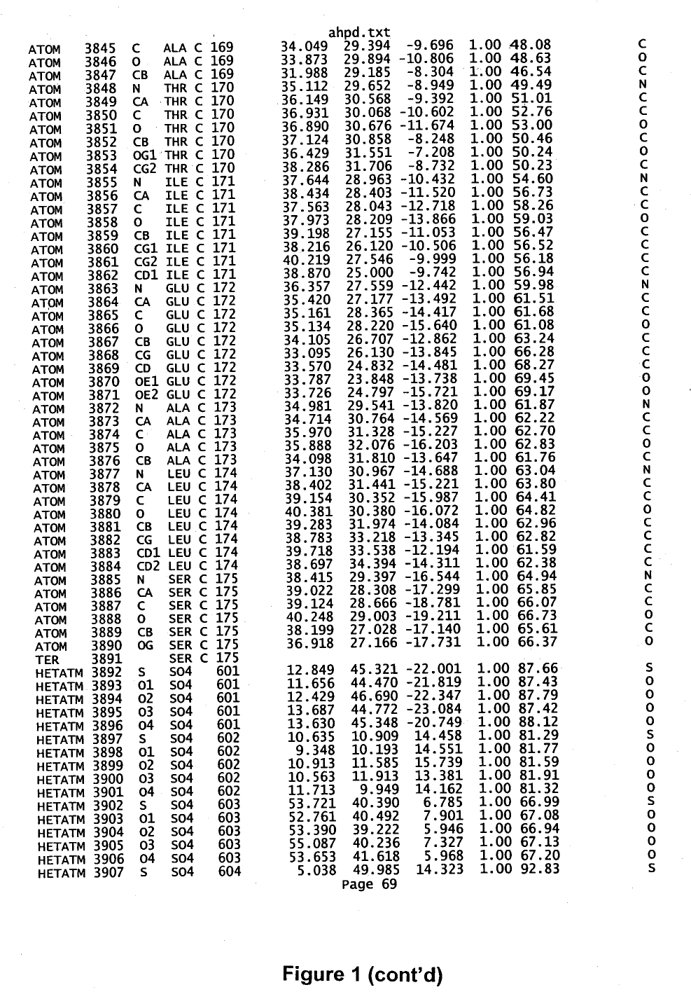

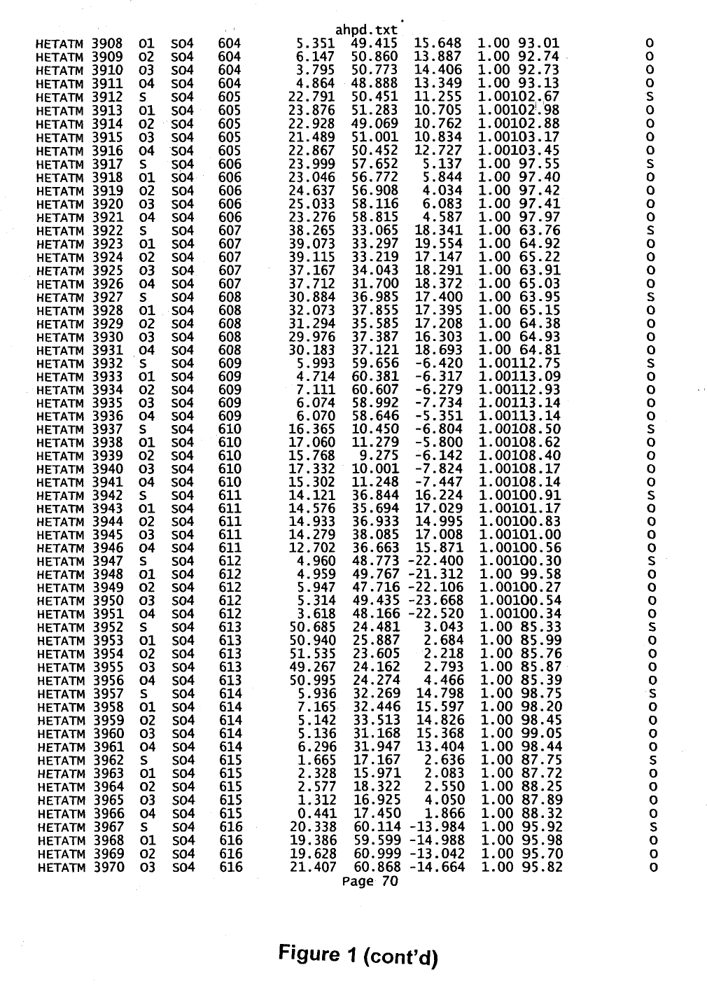

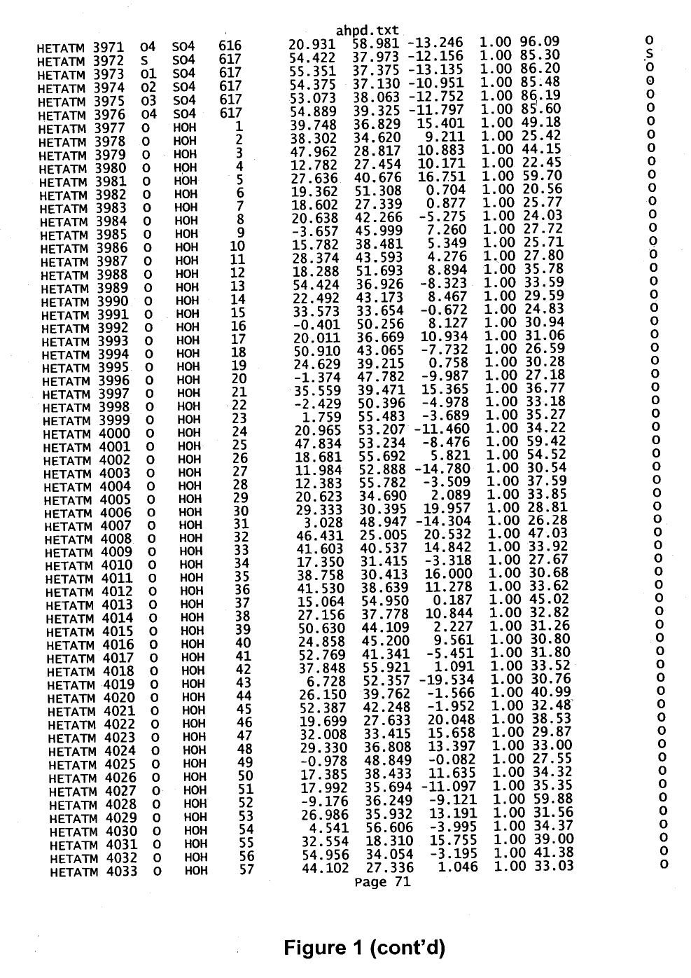

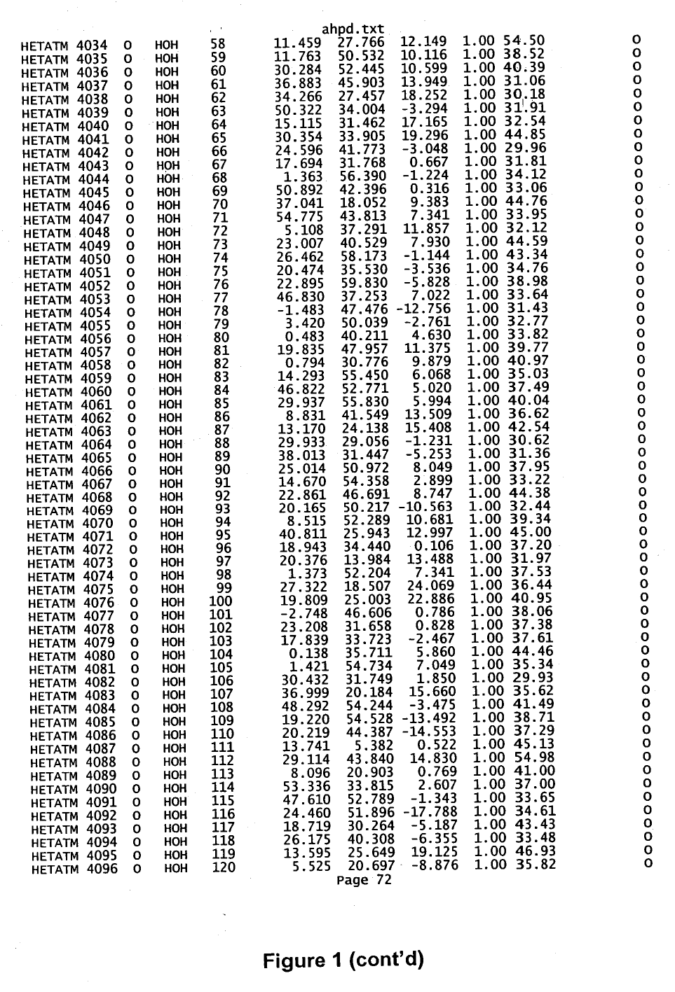

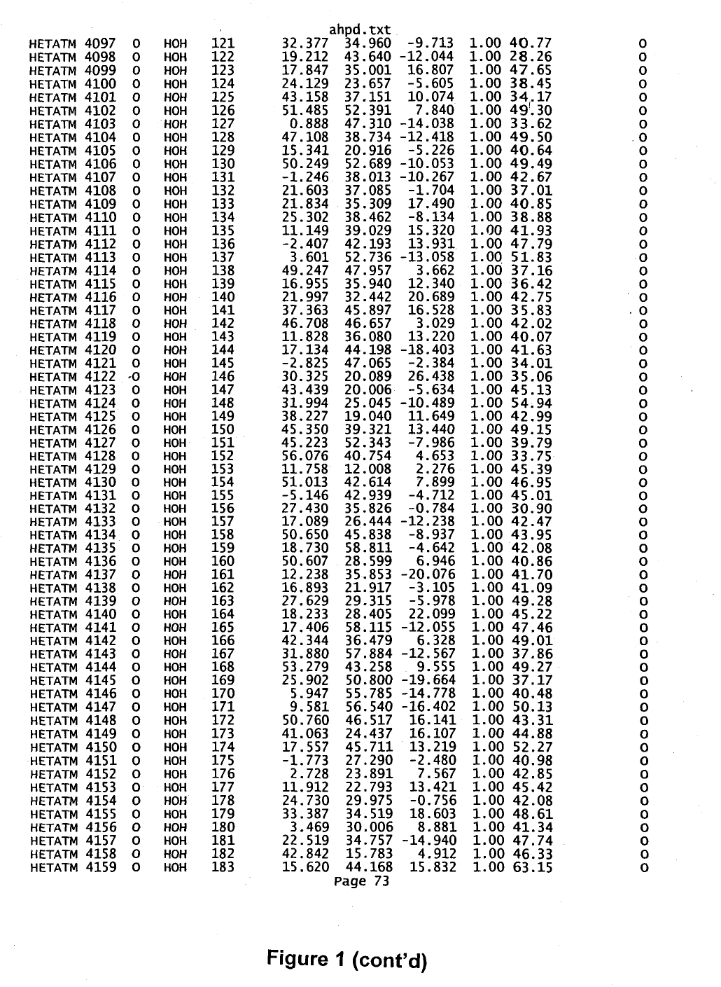

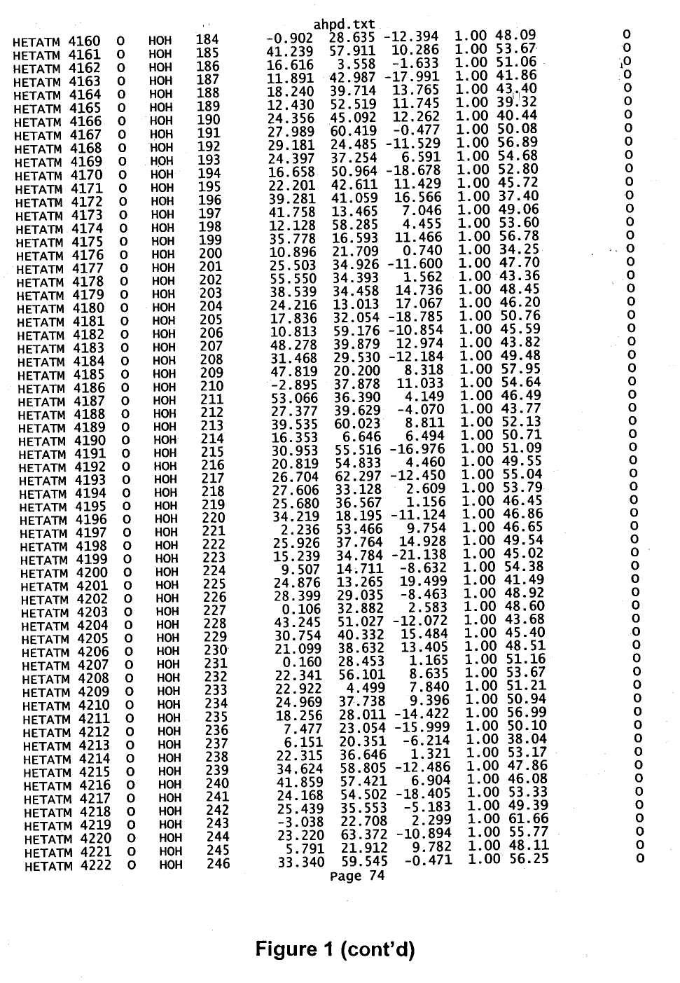

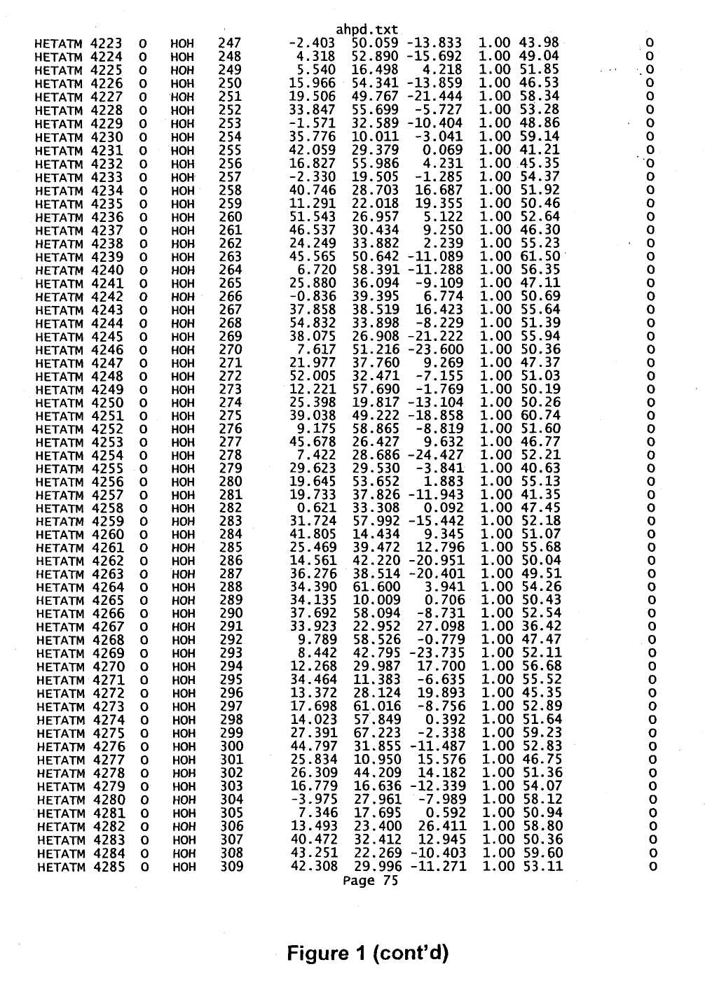

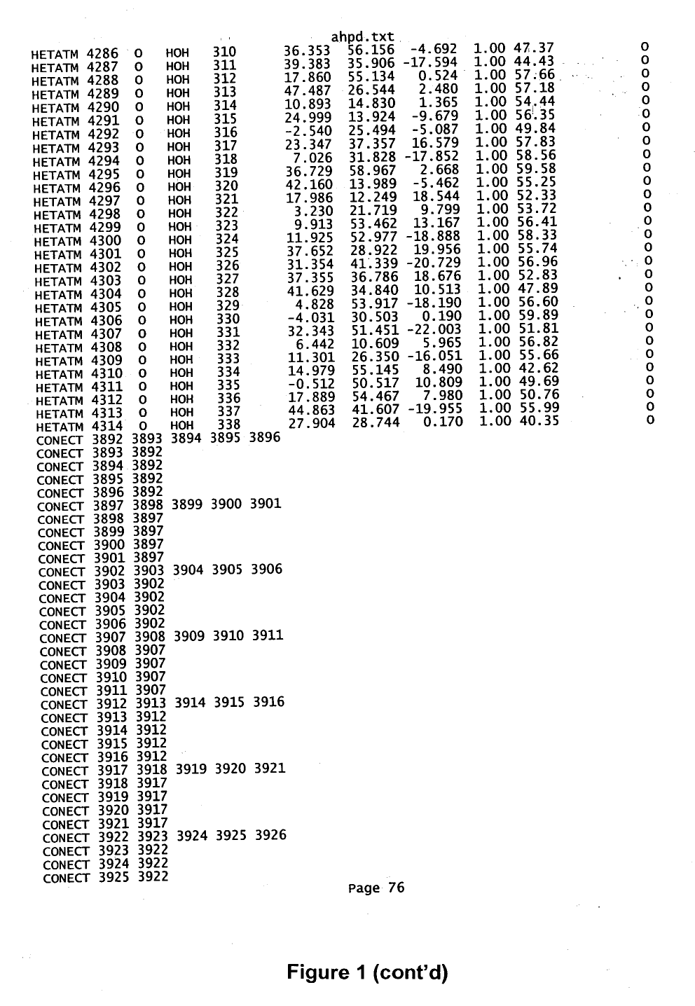

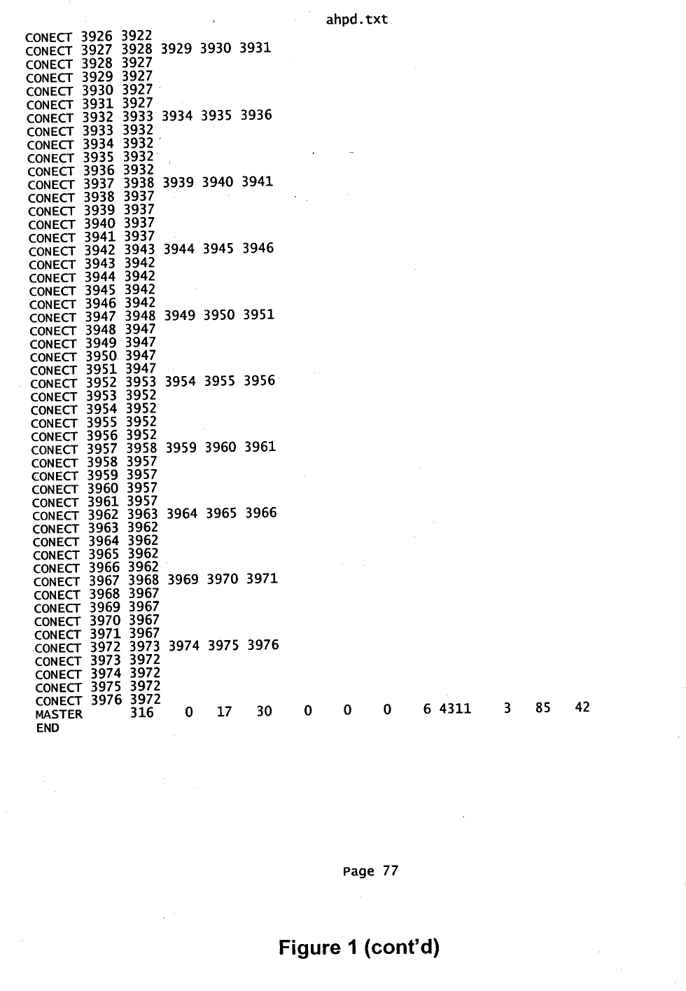

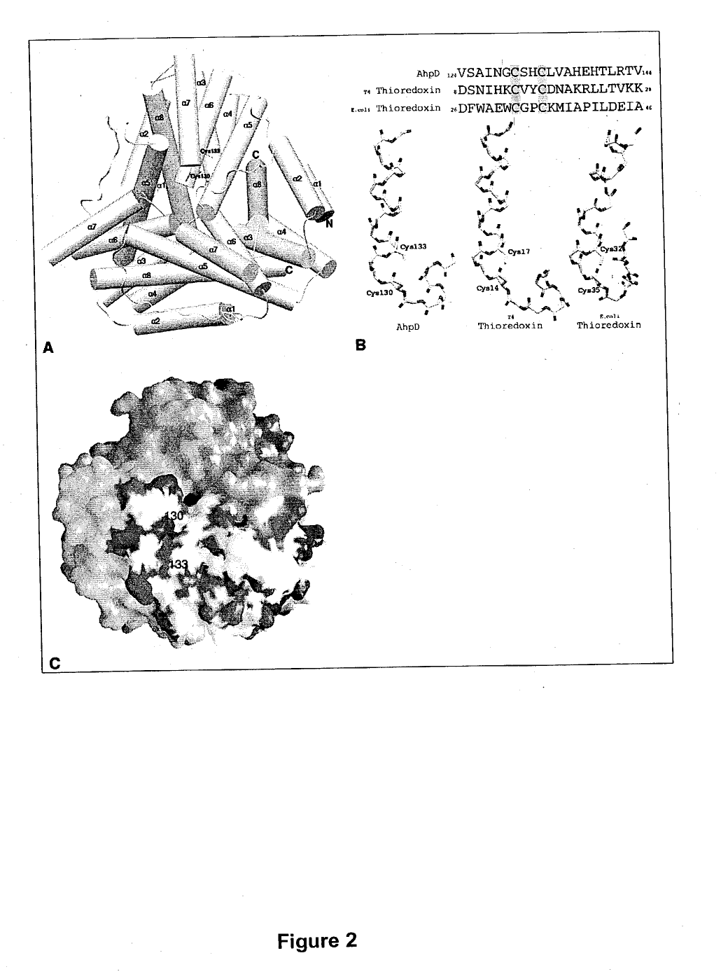

In another embodiment of the present invention, the inhibiting is achieved with a compound which binds to one or more molecular surfaces of the AhpD having a three dimensional crystal structure defined by the atomic coordinates set forth in FIG. 1. AhpD exists as a trimer with extensive interactions between each monomer (FIG. 2A). Each monomer of AhpD contains an active site with a pair of cysteine residues (Cys130 and Cys 133) that are found in a similar arrangement to those found in thioredoxin (FIG. 2B). The Cys130-Cys133 pair can undergo partial oxidation, similar to that observed for thioredoxin, is, thus, a potential binding site for a small molecule to block AhpD function.

In another embodiment of the present invention, the molecular surfaces of the AhpD include atoms surrounding representative active site cysteine residues 130 and/or 133. FIG. 2C depicts a ligand-binding molecular surface representation of AhpD, showing the three protomers as well as Cys130 and Cys133.

The molecular surface surrounding active site cysteine residue 130 can be defined by a set of atomic coordinates consisting of:

| | | | | | | | |
|---|---|---|---|---|---|---|---|
| ATOM | CG | ARG | A | 86 | 26.684 | 34.263 | 9.737 |
| ATOM | CD | ARG | A | 86 | 26.287 | 34.663 | 8.311 |
| ATOM | NH1 | ARG | A | 86 | 27.197 | 34.539 | 5.647 |
| ATOM | O | ARG | A | 86 | 26.997 | 33.147 | 12.918 |
| ATOM | NE | ARG | A | 88 | 33.177 | 31.048 | 17.082 |
| ATOM | NH2 | ARG | A | 88 | 34.982 | 32.389 | 17.508 |
| ATOM | CA | GLY | A | 89 | 28.223 | 33.948 | 16.115 |
| ATOM | C | GLY | A | 89 | 26.770 | 34.038 | 16.552 |
| ATOM | O | GLY | A | 89 | 26.456 | 34.664 | 17.568 |
| ATOM | CD1 | PHE | A | 90 | 23.685 | 34.988 | 13.747 |
| ATOM | CE1 | PHE | A | 90 | 23.618 | 35.735 | 12.567 |
| ATOM | CZ | PHE | A | 90 | 23.465 | 35.086 | 11.347 |
| ATOM | CB | GLU | A | 92 | 25.004 | 34.336 | 22.064 |
| ATOM | CG | GLU | A | 92 | 23.811 | 34.962 | 21.337 |
| ATOM | CD | GLU | A | 92 | 24.154 | 36.253 | 20.615 |
| ATOM | OE1 | GLU | A | 92 | 24.690 | 37.189 | 21.252 |
| ATOM | OE2 | GLU | A | 92 | 23.877 | 36.338 | 19.400 |
| ATOM | C | GLU | A | 92 | 27.302 | 33.404 | 22.076 |
| ATOM | O | GLU | A | 92 | 27.230 | 33.531 | 23.297 |
| ATOM | N | GLY | A | 93 | 28.321 | 32.798 | 21.482 |
| ATOM | CA | GLY | A | 93 | 29.422 | 32.280 | 22.275 |
| ATOM | OD1 | ASP | A | 96 | 31.819 | 31.356 | 19.922 |
| ATOM | OD2 | ASP | A | 96 | 32.705 | 32.998 | 21.057 |
| ATOM | O | GLY | A | 129 | 27.309 | 38.037 | 7.205 |
| ATOM | SG | CYS | A | 130 | 31.238 | 35.896 | 9.779 |
| ATOM | N | SER | A | 131 | 29.608 | 39.237 | 10.219 |
| ATOM | CB | SER | A | 131 | 28.953 | 40.371 | 12.262 |
| ATOM | OG | SER | A | 131 | 29.266 | 41.435 | 13.137 |
| ATOM | N | HIS | A | 132 | 31.421 | 38.650 | 12.395 |
| ATOM | CA | HIS | A | 132 | 32.637 | 38.217 | 13.077 |
| ATOM | CB | HIS | A | 132 | 32.540 | 36.743 | 13.482 |
| ATOM | CD2 | HIS | A | 132 | 34.060 | 36.247 | 15.526 |
| ATOM | NE2 | HIS | A | 132 | 35.322 | 35.720 | 15.649 |
| ATOM | O | HIS | A | 132 | 34.836 | 39.095 | 12.675 |
| ATOM | CG1 | VAL | A | 135 | 35.077 | 43.110 | 14.983 |
| ATOM | CG2 | VAL | A | 135 | 32.949 | 43.243 | 13.686 |
| ATOM | NH1 | ARG | B | 86 | 24.434 | 40.430 | 3.551 |
| ATOM | CD1 | PHE | B | 90 | 27.146 | 43.238 | 10.807 |
| ATOM | CE1 | PHE | B | 90 | 26.195 | 42.306 | 10.382 |
| ATOM | CZ | PHE | B | 90 | 26.429 | 41.551 | 9.242 |
| ATOM | O | PHE | B | 90 | 30.581 | 45.657 | 13.145 |
| ATOM | OE2 | GLU | B | 92 | 28.060 | 46.562 | 15.789 |
| ATOM | O | GLY | B | 129 | 21.817 | 41.212 | 5.427 |

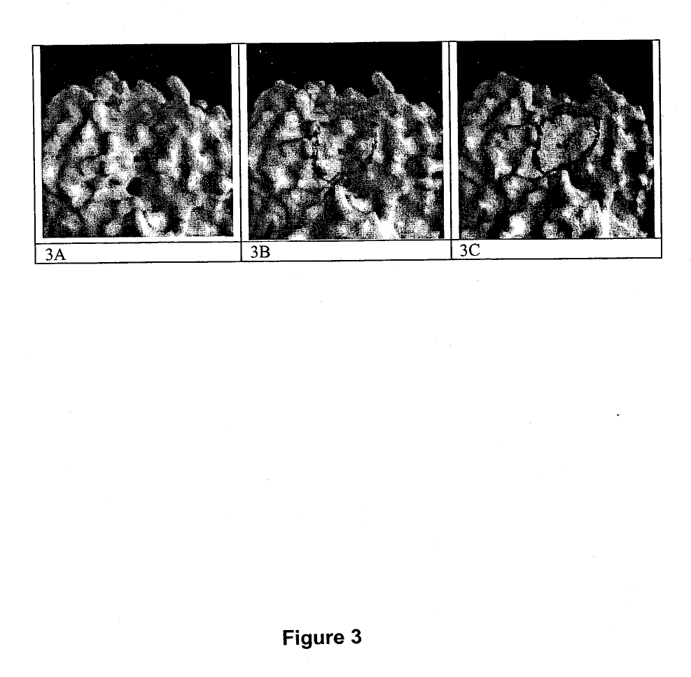

FIGS. 3A-C illustrate representative surfaces of AhpD that surround the active site cysteine residue, Cys 130, which can be targeted for potential inhibitor interactions. FIG. 3A shows a surface of AhpD with the Cys130 residue shaded. FIG. 3B shows a surface of AhpD with a surface scribed around the Cys130 residue. FIG. 3C shows a surface of AhpD with the scribed surface around the Cys130 filled in.

In another embodiment of the present invention, the molecular surface surrounding active site cysteine residue 133 is defined by a set of atomic coordinates consisting of:

| | | | | | | | |
|---|---|---|---|---|---|---|---|
| ATOM | ND2 | ASN | A | 81 | 38.756 | 31.671 | 8.422 |
| ATOM | CE1 | TYR | A | 85 | 36.018 | 31.618 | 14.046 |
| ATOM | CE2 | TYR | A | 85 | 36.646 | 31.599 | 11.723 |
| ATOM | CZ | TYR | A | 85 | 36.929 | 31.315 | 13.055 |
| ATOM | OH | TYR | A | 85 | 38.124 | 30.721 | 13.366 |
| ATOM | NH1 | ARG | A | 88 | 35.158 | 30.114 | 17.790 |
| ATOM | NH2 | ARG | A | 88 | 34.982 | 32.389 | 17.508 |
| ATOM | CB | PRO | A | 100 | 37.527 | 25.947 | 14.395 |
| ATOM | CG | PRO | A | 100 | 37.438 | 26.852 | 15.592 |
| ATOM | O | LEU | A | 102 | 41.472 | 25.358 | 10.446 |
| ATOM | N | MET | A | 104 | 43.466 | 26.552 | 7.835 |
| ATOM | CG | MET | A | 104 | 42.415 | 28.749 | 9.271 |
| ATOM | SD | MET | A | 104 | 41.163 | 29.814 | 10.015 |
| ATOM | CE | MET | A | 104 | 39.763 | 28.689 | 10.090 |
| ATOM | O | MET | A | 104 | 45.128 | 29.530 | 7.474 |
| ATOM | CA | ASN | A | 105 | 47.201 | 27.909 | 6.482 |
| ATOM | CG2 | ILE | A | 107 | 44.710 | 34.237 | 8.071 |
| ATOM | CD1 | ILE | A | 107 | 42.279 | 32.546 | 7.638 |
| ATOM | O | ILE | A | 107 | 47.536 | 34.661 | 6.821 |
| ATOM | CA | ALA | A | 108 | 49.252 | 32.809 | 7.921 |
| ATOM | CB | ALA | A | 108 | 49.613 | 31.745 | 8.959 |
| ATOM | O | ALA | A | 108 | 51.357 | 33.582 | 7.076 |
| ATOM | N | LYS | A | 114 | 50.989 | 40.121 | 4.422 |
| ATOM | CB | LYS | A | 114 | 49.659 | 39.422 | 6.349 |
| ATOM | CD | LYS | A | 114 | 50.479 | 37.681 | 7.965 |
| ATOM | CE | LYS | A | 114 | 51.122 | 36.318 | 8.106 |
| ATOM | NZ | LYS | A | 114 | 52.403 | 36.271 | 7.345 |
| ATOM | N | ALA | A | 115 | 49.121 | 42.363 | 4.988 |
| ATOM | CA | ALA | A | 115 | 48.224 | 43.514 | 4.993 |
| ATOM | CB | ALA | A | 115 | 49.021 | 44.816 | 5.065 |
| ATOM | CG | GLU | A | 118 | 45.071 | 40.454 | 7.074 |
| ATOM | OE2 | GLU | A | 118 | 44.218 | 38.267 | 7.520 |
| ATOM | CD2 | HIS | A | 132 | 34.060 | 36.247 | 15.526 |
| ATOM | CE1 | HIS | A | 132 | 35.771 | 35.402 | 14.447 |
| ATOM | NE2 | HIS | A | 132 | 35.322 | 35.720 | 15.649 |
| ATOM | O | HIS | A | 132 | 34.836 | 39.095 | 12.675 |
| ATOM | SG | CYS | A | 133 | 34.765 | 35.332 | 9.372 |
| ATOM | CG1 | VAL | A | 135 | 35.077 | 43.110 | 14.983 |
| ATOM | CA | ALA | A | 136 | 38.186 | 40.749 | 12.941 |
| ATOM | CB | ALA | A | 136 | 38.197 | 39.238 | 12.924 |
| ATOM | O | ALA | A | 136 | 40.246 | 41.806 | 12.333 |
| ATOM | ND1 | HIS | A | 137 | 40.517 | 38.915 | 8.235 |
| ATOM | CE1 | HIS | A | 137 | 40.229 | 37.629 | 8.163 |
| ATOM | NE2 | HIS | A | 137 | 38.923 | 37.502 | 8.009 |
| ATOM | CB | HIS | A | 139 | 40.410 | 44.362 | 14.769 |
| ATOM | CG | HIS | A | 139 | 41.301 | 44.548 | 15.963 |
| ATOM | CD2 | HIS | A | 139 | 42.219 | 43.729 | 16.529 |
| ATOM | CE1 | HIS | A | 139 | 42.194 | 45.593 | 17.687 |
| ATOM | NE2 | HIS | A | 139 | 42.759 | 44.403 | 17.601 |
| ATOM | OG1 | THR | A | 140 | 43.391 | 41.000 | 12.322 |
| ATOM | CG2 | THR | A | 140 | 45.224 | 41.726 | 10.943 |
| ATOM | CB | THR | A | 143 | 46.647 | 45.739 | 14.859 |
| ATOM | OG1 | THR | A | 143 | 46.606 | 44.614 | 13.978 |
| ATOM | CG2 | THR | A | 143 | 45.377 | 45.766 | 15.704 |
| ATOM | O | THR | A | 143 | 49.154 | 47.078 | 13.758 |
| ATOM | CA | VAL | A | 144 | 49.134 | 46.659 | 11.050 |
| ATOM | CB | VAL | A | 144 | 49.041 | 45.516 | 10.013 |
| ATOM | CG1 | VAL | A | 144 | 48.891 | 44.185 | 10.726 |
| ATOM | O | VAL | A | 144 | 50.259 | 48.007 | 9.412 |

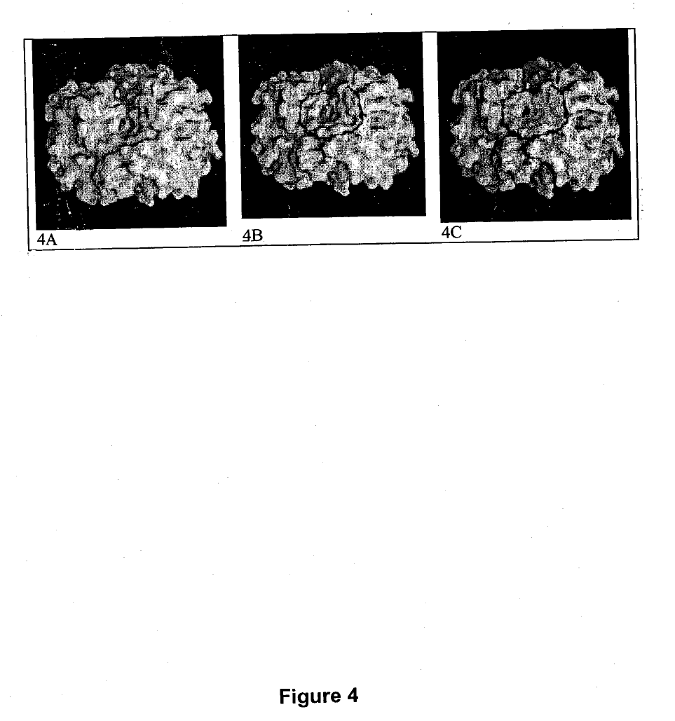

FIGS. 4A-C illustrate representative surfaces of AhpD that surround the active site cysteine residue, Cys 133, which can be targeted for potential inhibitor interactions. FIG. 4A shows a surface of AhpD with the Cys133 residue shaded. FIG. 4B shows a surface of AhpD with a surface scribed around the Cys133 residue. FIG. 4C shows a surface of AhpD with the scribed surface around the Cys133 filled in.

The present invention also relates to a method of treating tuberculosis in a subject. The method involves inhibiting AhpD in the subject under conditions effective to make the pathogen susceptible to antimicrobial reactive nitrogen intermediates or reactive oxygen intermediates.

In another embodiment of the present invention, the inhibiting is achieved with a compound which binds to one or more molecular surfaces of the AhpD having a three dimensional crystal structure defined by the atomic coordinates set forth in FIG. 1. The molecular surfaces of the AhpD can include atoms surrounding representative active site cysteine residues 130 and/or 133. In other embodiments of the present invention, the molecular surfaces of AhpD surrounding active site cysteine residues 130 and 133 can be defined by the sets of atomic coordinates as described above.

Another aspect of the present invention relates to a method for identifying candidate compounds suitable for treatment or prevention of tuberculosis in a subject. The method first involves contacting AhpD with a compound. Then, those compounds which bind to the AhpD are identified as candidate compounds suitable for treatment or prevention of tuberculosis in a subject.

The present invention also relates to a method of preventing onset of tuberculosis in a subject infected with Mycobacterium tuberculosis. The method involves inhibiting dihydrolipoamide dehydrogenase in Mycobacterium tuberculosis in the subject under conditions effective to make the pathogen susceptible to antimicrobial reactive nitrogen intermediates or reactive oxygen intermediates.

In another embodiment of the present invention, the dihydrolipoamide dehydrogenase is encoded by an RV0462 gene in Mycobacterium tuberculosis.

Dihydrolipoamide dehydrogenase (Lpd) of M. tuberculosis (Rv0462; NCBI# 7431875) lies in a presumptive operon with several unannotated hypothetical proteins. Lpd is a FAD-containing NADH-dependent oxidoreductase that plays an essential role in intermediary metabolism as the E3 component of pyruvate dehydrogenase (PDH), α-ketoglutarate dehydrogenase (KGDH) and branched-chain α-keto acid dehydrogenase (BCKADH) complexes (Perham, Annu. Rev. Biochem., 69: 961 (2000), which is hereby incorporated by reference in its entirety). In these complexes, Lpd regenerates the dihydrolipoic (6,8-dithiooctanoic acid) acceptors covalently attached to ε-amino groups of lysine residues on the “swinging arm(s)” of the E2 acetyl(succinyl)transferase component (Perham, Annu. Rev. Biochem., 69: 961 (2000), which is hereby incorporated by reference in its entirety).

The only previously demonstrated function of Lpd was to serve as the E3 component of PDH, KGDH and BCKADH complexes. However, homologs of Lpd are more widely distributed than are the dehydrogenase complexes themselves, being found without other components in some anaerobes, archaebacteria, and trypanosomatids (Perham, Annu. Rev. Biochem., 69: 961 (2000); Danson, Biochem. Soc. Trans., 16: 87 (1988), which are hereby incorporated by reference in their entirety). This distribution suggests the evolutionary conservation of a novel function of Lpd. Lpd may constitute part of a peroxiredoxin-based peroxidase-peroxynitrite reductase in organisms besides M. tuberculosis, perhaps involving AhpD-equivalents with a thioredoxin-like fold. Others have suggested an antioxidant function for Lpd related to the role of free lipoic acid as an antioxidant (Haramaki et al., Free Radic. Biol. Med., 22: 535 (1997), which is hereby incorporated by reference in its entirety).

Another aspect of the present invention relates to a method of treating tuberculosis in a subject. The method involves inhibiting dihydrolipoamide dehydrogenase in Mycobacterium tuberculosis in the subject under conditions effective to make the pathogen susceptible to antimicrobial reactive nitrogen intermediates or reactive oxygen intermediates.

Yet another aspect of the present invention relates to a method for identifying candidate compounds suitable for treatment or prevention of tuberculosis in a subject. The method first involves contacting a dihydrolipoamide dehydrogenase in Mycobacterium tuberculosis with a compound. Then, those compounds which bind to the dihydrolipoamide dehydrogenase in Mycobacterium tuberculosis are identified as candidate compounds suitable for treatment or prevention of tuberculosis in a subject.

The present invention also relates to a method of preventing onset of tuberculosis in a subject infected with Mycobacterium tuberculosis. The method involves inhibiting dihydrolipoamide succinyltransferase in Mycobacterium tuberculosis in the subject under conditions effective to make the pathogen susceptible to antimicrobial reactive nitrogen intermediates or reactive oxygen intermediates.

In another embodiment of the present invention, the dihydrolipoamide succinyltransferase is encoded by a sucB (RV2215; NCBI# 1709443) gene in Mycobacterium tuberculosis.

Dihydrolipoamide succinyltransferase (SucB) is annotated as the E2 component of KGDH. Immunochemistry and bioinformatics suggests that SucB appears to be the only lipoylated protein in M. tuberculosis H37Rv. If so, then SucB presumably sustains both the PDH and KGDH activities that were detected in M. tuberculosis 30-40 years ago but only partially purified (Murthy et al., Amer. Rev. Resp. Dis., 108: 689 (1973), which is hereby incorporated by reference in its entirety). E. coli has organized into operons its genes encoding PDH (aceE, aceF, lpd) (Stephens et al., Eur. J. Biochem., 133: 481 (1983), which is hereby incorporated by reference in its entirety) and KGDH (sucA, sucB, sucC, sucD) (Spencer et al., Eur. J. Biochem., 141: 361 (1984), which is hereby incorporated by reference in its entirety). No such gene clusters are evident in M. tuberculosis. Near sucB lie lipB (lipoate protein ligase) and lipA (lipoate synthase), which may function to lipoylate SucB. M. tuberculosis's sucA (E1 homolog of KGDH) is transcribed divergently elsewhere.

Another aspect of the present invention relates to a method of treating tuberculosis in a subject. The method involves inhibiting dihydrolipoamide succinyltransferase in Mycobacterium tuberculosis in the subject under conditions effective to make the pathogen susceptible to antimicrobial reactive nitrogen intermediates or reactive oxygen intermediates.

Yet another aspect of the present invention relates to a method for identifying candidate compounds suitable for treatment or prevention of tuberculosis in a subject. The method first involves contacting a dihydrolipoamide succinyltransferase in Mycobacterium tuberculosis with a compound. Then, those compounds which bind to the dihydrolipoamide succinyltransferase in Mycobacterium tuberculosis are identified as candidate compounds suitable for treatment or prevention of pathogen infection in a subject.

In other embodiments of the present invention, the inhibiting of AhpD, dihydrolipoamide dehydrogenase, or dihydrolipoamide succinyltransferase can be carried out by administering an inhibitor of AhpD, dihydrolipoamide dehydrogenase, or dihydrolipoamide succinyltransferase orally, intradermally, intramuscularly, intraperitoneally, intravenously, subcutaneously, or intranasally. The inhibitor compounds of the present invention may be administered alone or with suitable pharmaceutical carriers, and can be in solid or liquid form, such as tablets, capsules, powders, solutions, suspensions, or emulsions.

The inhibitor compounds may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The inhibitor compounds may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The present invention also relates to a method of producing an AhpD crystal suitable for X-ray diffraction. The method first involves subjecting a solution of AhpD under conditions effective to grow a crystal of AhpD to a size suitable for X-ray diffraction. Then, an AhpD crystal suitable for X-ray diffraction is obtained.

Current approaches to macromolecular crystallization are described in McPherson, Eur. J. Biochem., 189:1-23 (1990), which is hereby incorporated by reference in its entirety.

In one embodiment of the present invention, the AhpD crystal has space group P6₅22 and unit cell dimensions of approximately a=108.3 Å, b=108.3 Å, and c=233.6 Å such that the three dimensional structure of the crystallized AhpD can be determined to a resolution of about 2.0 Å or better.

In another embodiment of the present invention, the crystallization occurs in hanging drops using a vapor diffusion method (Hampel et al., Science 162:1384 (1968), which is hereby incorporated by reference in its entirety).

In another embodiment, the present invention is a AhpD crystal produced by the method of the present invention involving subjecting a solution of AhpD under conditions effective to grow a crystal of AhpD to a size suitable for X-ray diffraction, and obtaining an AhpD crystal suitable for X-ray diffraction.

The present invention also relates to a method for designing a compound suitable for treatment or prevention of tuberculosis in a subject. The method first involves providing a three-dimensional structure of a crystallized AhpD. Then, a compound having a three-dimensional structure which will bind to one or more molecular surfaces of the AhpD is designed. In other embodiments, the present invention includes a compound designed by the method of the present invention and a pharmaceutical composition having the compound of the present invention and a pharmaceutical carrier. In another embodiment of the present invention, the three dimensional structure of a crystallized AhpD is defined by the atomic coordinates set forth in FIG. 1. The molecular surfaces of the AhpD can include atoms surrounding representative active site cysteine residues 130 and/or 133. In other embodiments of the present invention, the molecular surfaces of AhpD surrounding active site cysteine residues 130 and 133 can be defined by the sets of atomic coordinates as described above.

Another aspect of the present invention relates to a compound suitable for treatment or prevention of tuberculosis in a subject. The compound has a three-dimensional structure which will bind to one or more molecular surfaces of the AhpD having a three dimensional crystal structure defined by the atomic coordinates set forth in FIG. 1.

### EXAMPLES

The following examples are provided to illustrate embodiments of the present invention but are by no means intended to limit its scope.

### Example 1

Effect of AhpD on AhpC Peroxidase Activity

In order to search for an AhpC reductase in M. tuberculosis, it was examined whether pure AhpC (Bryk et al., Nature, 407: 211 (2000), which is hereby incorporated by reference in its entirety) could reduce H₂O₂ when supplemented with lysate from M. tuberculosis H37Rv. M. tuberculosis H37Rv and M. bovis BCG lysates were prepared in 25 mM KPi, 1 mM EDTA, 1 mM PMSF by bead beater from log phase cultures. The AhpD open reading frame (ORF) was amplified by PCR with engineered 5′ NdeI and 3′ NheI sites and cloned into pET11c. Expression was induced in E. coli BL21(DE3) with 1 mM IPTG. AhpD was purified to homogeneity by phenyl Sepharose, Q Sepharose and Sephadex G200 chromatography. AhpD C130S and AhpD C133S were generated using QuikChange Site-Directed Mutagenesis Kit (Stratagene, La Jolla, Calif.).

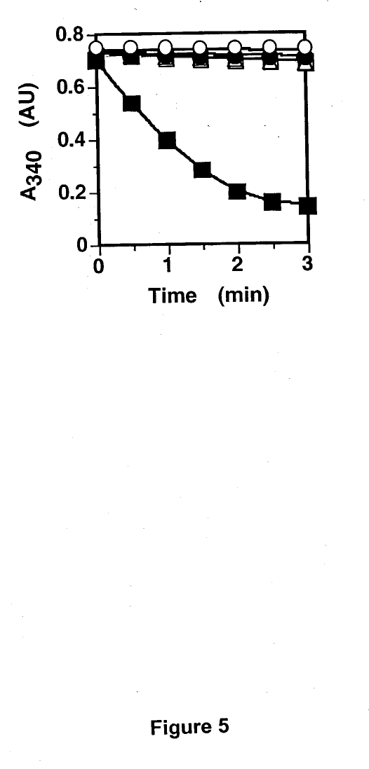

Neither NADH nor NADPH supported peroxidase activity by AhpC in the presence of lysate from M. tuberculosis H37Rv (FIG. 5). However, the further addition of pure AhpD produced a robust, NADH-dependent, cyanide-insensitive peroxidase activity. Single cysteine mutants (AhpD C130S; AhpD C133S) could not substitute for wild type AhpD. AhpD by itself showed minimal peroxidatic activity, as previously reported (Hillas et al., J. Biol. Chem., 275: 18801 (2000), which is hereby incorporated by reference in its entirety). On the scale of the reaction in FIG. 5, the contribution of AhpD alone was imperceptible.

### Example 2

Crystallization and Structure Determination of AhpD

To gain insight into the function of AhpD and set constraints on the identity of the elements that reduce it, AhpD was crystallized and its structure was solved at 2.0 Å resolution by X-ray diffraction. 96-well crystallization trials were conducted that produced diffraction quality crystals in several conditions. AhpD crystals of superior diffraction quality were grown by hanging drop vapor diffusion against a well solution containing ammonium sulfate from 1.5M to 2.5M to a final size of 0.3×0.3×0.4 mm. The data were obtained from AhpD crystallized in space group P6₅22 (a=b=108.3 Å, c=233.6 Å, α=β=90°γ=120°). Diffraction data collection was accomplished with cryo-preserved crystals (25% glycerol). Crystals of native and thimerosal derivatives were diffracted at beam line X4A at the National Synchrotron Light Source and a laboratory copper Kα source (Rigaku RU200) equipped with Osmic multi-layer optics and a Raxis-IV imaging plate detector, respectively. Data was processed with DENZO and SCALEPACK (Otwinowski et al., Meth. Enzym. 276:307 (1997), which is hereby incorporated by reference in its entirety), and input to SOLVE (Terwilliger et al., Acta Crystallogr., D55:849 (1999), which is hereby incorporated by reference in its entirety), SHARP, and the CCP4 suite (Collaborative Computational Project, Acta Crystallogr., D50:760 (1994), which is hereby incorporated by reference in its entirety) to calculate a 2.6 Å phase set. Density modification and phase extension to 2.0 Å was accomplished with Arp/Warp (Lamzin et al., Acta Crystallogr., D49:129 (1993), which is hereby incorporated by reference in its entirety). Approximately 80% of the polypeptide chain was traced automatically into the electron density maps using Arp/Warp. The resulting chains were corrected and modified using the program O (Springer et al., Infect. Immun. 69, 5967 (2001), which is hereby incorporated by reference in its entirety) (Table 1). The model was initially refined using Refmac (Murshudov et al., Acta Crystallogr., D53:240 (1997), which is hereby incorporated by reference in its entirety) and subsequently with CNS (Brunger et al., Acta Crystallog., D54:905 (1998), which is hereby incorporated by reference in its entirety). The model contained 521 amino acid residues excluding the N-terminal amino acid from all 3 protomers, and amino acid 176 from C-terminal end of each protomer (Table 1). Lipoic acid and H₂O₂ soaks utilized 1 mM solutions dissolved in mother liquor. Hydrogen peroxide treatment was complete after 5 doses of a final 1 mM concentration of H₂O₂ over 2 hours. Crystals were incubated in these solutions, cryo-preserved as described previously, and diffracted using the laboratory x-ray source. The final model had excellent geometry with 95.7%, 4.3%, and 0.0% of residues in favorable, allowed, and generously allowed regions of the Ramachandran plot, respectively. Coordinates and structure factors are deposited in the Protein Data Bank for native AhpD (PDB Accession No. 1KNC).

TABLE 1
Summary of Crystallographic Analysis

| | Multiple | |
|---|---|---|
| | Isomorphous Replacement | |
| | Native (high) | 1 mM Thimerosal |
| dMin/λ (Å) | 20-2.0/0.9787 | 20-2.4/1.5418 |
| No. of sites | — | 3 |
| Rsym (%)a overall (outer shell) | 7.7 (31.3) | 4.1 (21.6) |
| Coverage (%) overall (outer shell) | 99.7 (98.9) | 93.8 (70.7) |
| I/σ (I) overall (outer shell) | 18.0 (2.7) | 9.1 (2.4) |
| Reflections (total/unique) | 1515540/55072 | 869561/30651 |
| Phasing statistics | 20-2.6 Å | |
| MFID (%) | 17.5 | |
| Overall Phasing power | 0.73/0.75 | |
| (centric/acentric) | | |
| Mean FOM (centric/acentric) | 0.29/0.33 | |
| Mean FOM after wARP (20-2.0 Å) | 0.92 | |

| Refinement | |
|---|---|
| Resolution range (Å) | 20-2.0 |
| # Reflections (work/free) > 0.0 σ | 52321/2751 |
| Total #atoms/#water/#SO4 atoms | 4314/338/85 |
| R/Rfree | 0.216/0.244 |
| Rmsd bond(Å)/angles(°) | 0.005/0.929 |
| Rmsd B(Å²) main chain/side chain | 1.144/1.816 |

Rsym = Σ|I − <I>|/ΣI, where I = observed intensity, and <I> = average intensity
R, R based on 95% of the data used in refinement;
Rfree, R based on 5% of the data withheld for the cross-validation test.
MFID (mean fractional isomorphous difference) = Σ∥Fph| − |Fp∥/Σ|Fp|, where Fp = protein structure factor amplitude and |Fph| = heavy-atom derivative structure factor amplitude
Phasing power = root-mean-square (|Fh|/E, where |Fh| = heavy-atom structure factor amplitude and
E = residual lack of closure error.
Rc = Σ∥Fh(obs)| − |Fh(calc)∥/Σ|Fh(obs)| for centric reflections where |Fh(obs)| = observed heavy atom structure factor amplitude, and |Fh(calc)| = calculated heavy-atom structure factor amplitude.
Mean FOM = Combined figure of merit.
Rmsd = root-mean-square deviation of bond lengths, angles, and B factors.

The AhpD protomer was nearly all helical except for residues 93-113, which adopt an extended conformation between protomer contacts (FIG. 2A). Trimerization is sustained by interactions between helices α2, α8, α6, and α7 from one protomer with helix α5 and resides 96-104 from an adjacent protomer. The protomers interact via hydrophobic contacts, hydrogen bonds and salt bridges. The AhpD fold appeared to be unique; no structural homology was revealed using protomer or trimer models in a search with the programs DALI or PFAM (Holm et al., J. Mol. Biol., 233:123 (1993), which is hereby incorporated by reference in its entirety). However, three distinct thioredoxin-like AhpD active sites (one per promoter) contained conserved cysteine residues (Cys130 and Cys133) located at the N-terminal end of helix α7, and structure-based alignment revealed similarity with thioredoxin within this site (FIG. 2B). Cys133 is accessible to interactions with large molecules at the base of a cleft found within each protomer (FIG. 2C), while Cys130 is partially buried within the fold and appeared to be blocked from potential ligand interactions by protomer-protomer contacts. The active site cleft is lined almost entirely by polar and hydrophobic side chains. This suggested that the active site could be accessed by a redox-active moiety offered via a hydrophobic arm. The cleft is also large enough to serve as a potential ligand-binding pocket for AhpC (FIG. 2C).

### Example 3

Purification of Activity from M. tuberculosis Lysate That Supports the Peroxidase Function of AhpC plus AhpD

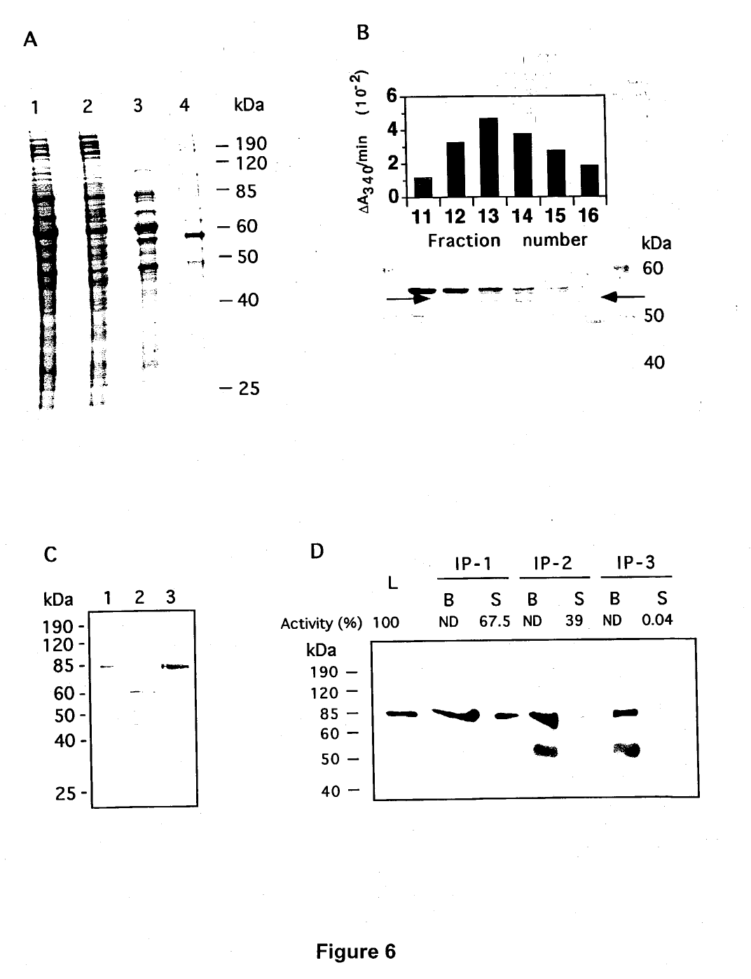

An activity from M. tuberculosis lysate that could support the peroxidase function of AhpC plus AhpD was successfully purified through fractional ammonium sulfate precipitation and anion exchange (FIG. 6A). Further hydrophobic interaction or nucleotide affinity chromatography led to complete loss of activity, raising the possibility that there were two separable active principles. Therefore, the activity profile of fractions from Q Sepharose were compared with their Coomassie blue-stained protein banding pattern. The abundance of 3 polypeptide bands most closely matched activity (FIG. 6B). These bands were isolated from SDS-PAGE, digested with trypsin and peptide mass-fingerprinted (Mann et al., Biol. Mass Spectrom. 22, 338 (1993); Erjument-Bromage et al., J. Chromatogr. 826, 167 (1998), which are incorporated by reference in their entirety). Two of the 3 bands corresponded to hypothetical protein Rv0462 (NCBI# 7431875), a homolog of dihydrolipoamide dehydrogenase (Lpd). The identification was based on 8 tryptic matches with an average difference of 0.006 absolute mass units (amu) between observed and predicted masses, covering 30% of the coding sequence.

To confirm that Lpd could replace mycobacterial lysate, Lpd (0.2 units, Sigma, St. Louis, Mo.) from bovine intestinal mucosa was added to AhpC+AhpD+NADH. H₂O₂-dependent consumption of NADH ensued, but only when the reaction was further supplemented with 50 μM lipoic acid. To evaluate the potential responsiveness of AhpD to this cofactor, AhpD crystals were exposed to oxidized lipoamide or H₂O₂. Diffraction analysis revealed that the 2 AhpD cysteines could be more readily oxidized by lipoamide than H₂O₂. His132 underwent a rotamer change in which the imidazole near Cys133 in reduced AhpD now pointed away, while on average the sulfhydryls of Cys133 and Cys130 moved closer together.

### Example 4

Identification of Lipoylated Proteins in Mycobacterial Lysates

Though free lipoic acid sustained the peroxidase activity of AhpC+AhpD+bovine Lpd, lipoic acid in cells is almost all protein-bound. Thus, mycobacterial lysate may also supply a lipoylated protein. Indeed, immunoblot of M. tuberculosis lysate with α-lipoic acid antibody (FIG. 6C) revealed a single lipoylated polypeptide, p85. This species was enriched in the active peak from Q Sepharose (FIG. 6B). Applied to a lysate of M. bovis BCG, the same antibody revealed 2 smaller lipoylated species, p46 and p60. The BCG lysate was not able to complement H₂O₂-dependent AhpC activity in the presence of AhpD. BCG's p46 and p60 may represent degradation products of p85 or a different set of lipoylated proteins. Nonetheless, the presence of p85 correlated with activity.

To confirm that the lipoylated protein detected by the anti-lipoic acid antibody contributed to peroxidase activity, the same antibody was used to immunodeplete the protein from M. tuberculosis lysate. Lysates (1.5 mg total protein) were incubated with α-lipoic acid Ab (1:200) overnight at 4° C. and immune complexes were precipitated with protein G agarose. Beads were washed 3 times in 0.5 ml of 50 mM KPi, pH 7.0, 1 mM EDTA, 150 mM NaCl, 10% glycerol, 0.1% Tween-20 and boiled with 25 μl sample buffer. Samples were analyzed by 10% SDS-PAGE and visualized by Western Blot with the same antibody (1:5,000). Immunodepletion was carried out in stages to seek a concentration-response relationship. Supernates (50 μl) were tested for residual activity as in FIG. 5. Gradual depletion of p85 led to a corresponding and eventually complete loss of ability of the lysate to support H₂O₂-dependent AhpC activity in the presence of AhpD (FIG. 6D).

Peptide mass fingerprinting identified p85 as a homolog of dihydrolipoamide succinyltransferase, annotated as the E2 component of KGDH (Rv2215; NCBI# 1709443). This identification was based on 15 tryptic matches with an average difference of 0.036 amu between observed and predicted masses, covering 35% of the coding sequence. BLAST search of the M. tuberculosis H37Rv genome with a consensus lipoylation sequence identified the same protein, annotated as sucB (Cole et al., Nature, 393: 537-544 (1998); http://www.sanger.ac.uk/Projects/M_tuberculosis/, which are hereby incorporated in their entirety). The sucB gene encodes 2 lipoylation consensus sequences, DEPLVEVSTDKVDTEIPSP (SEQ ID NO: 1), suggesting that SucB is most likely lipoylated at Lys43 and Lys 162.

### Example 5

Reconstitution of AhpC Enzymatic Activity with Recombinant Proteins

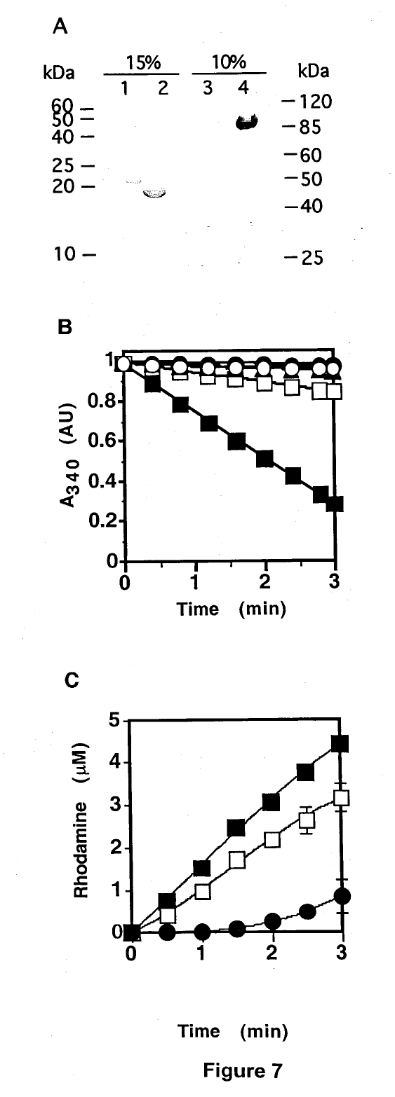

To reconstitute peroxidase activity solely with mycobacterial proteins, Lpd and SucB ORFs were amplified by PCR from M. tuberculosis H37Rv genomic DNA. Lpd primers were with engineered 5′ NdeI (5′GGGTAGGGCATATGACCCACTATGACGTCG3′; SEQ ID NO: 2) and 3′ NheI (5′GCTCGCGCTAGCCGTCATGAGCCG3′; SEQ ID NO: 3) sites. SucB primers contained 5′ NdeI (5′GGAGTCAACACATATGGCCTTCTCCG3′; SEQ ID NO: 4) and 3′ BamHI (5′GCGATCGGATCCACGGCGTTGG3′; SEQ ID NO: 5) sites. Fragments were cloned into pET11c digested with corresponding sets of enzymes. Protein expression was induced in E. coli BL21 (DE3) with 1 mM IPTG. Lpd was purified to homogeneity from inclusion bodies by Q Sepharose chromatography. SucB expression was induced in cells supplemented with 200 μM lipoic acid to ensure lipoylation. SucB was purified by Q Sepharose and avidin agarose chromatography, eluting from the latter column with 5 mM lipoic acid, which was subsequently dialyzed out. (FIG. 7A).

Lpd, SucB, AhpD, and AhpC together sustained brisk H₂O₂-dependent oxidation of NADH (FIG. 7B). No activity was observed when Lpd, AhpD, or AhpC was omitted. In the absence of SucB, the system operated at about 30% of the rate observed in the presence of SucB. The complete system sustained slightly higher levels of activity when cumene and tert-butyl hydroperoxides were substrates in place of H₂O₂. Thus, these four proteins constitute a peroxidase active toward both hydrogen and alkyl peroxides.

To find out if the endogenous 4-component peroxidase from M. tuberculosis could serve as a peroxynitrite reductase, peroxynitrite was infused into a reaction mixture containing pure recombinant Lpd, SucB, AhpD, AhpC, and NADH (FIG. 7C). Peroxynitrite was infused from a stock solution of 100 □M in 3 mM NaOH at a rate of 200 μl/min for 3 minutes. Aliquots of 50 μl were withdrawn every 30 sec and rhodamine absorbance was measured at 500 nm. The pH of the reaction did not change after peroxynitrite infusion. Rhodamine formation was calculated by ε₅₀₀=78,800 M⁻¹cm⁻¹. Results are means ±S.D. of triplicates. The system efficiently metabolized peroxynitrite as assessed by protection of dihydrorhodamine from oxidation. Peroxynitrite reductase activity under these conditions continued for 3 min, after which NADH was exhausted. Given the rate of reaction of M. tuberculosis AhpC with peroxynitrite (1.33×10⁶ M⁻¹sec⁻¹) (Bryk et al., Nature, 407: 211 (2000), which is hereby incorporated by reference in its entirety) and the 20-fold molar excess of peroxynitrite over AhpC in this experiment, sustained protection of dihydrorhodamine clearly reflected a catalytic cycle. Under the same conditions, the heterologous system of M. tuberculosis AhpC with AhpF from S. typhimurium afforded much weaker protection (FIG. 7C). Thus, AhpC+AhpD+SucB+Lpd constitute an endogenous mycobacterial peroxynitrite reductase.

### Example 6

Screening for Potential Inhibitor Compounds

The screen was performed in Falcon Microtest 384-well 30 μl assay plates using the DTNB (5,5′-dithiobis-(2-nitrobenzoic acid)) assay.

The protein mixture (200 nM Lpd, 350 nM SucB, 36 nM AhpD) was dispensed into each well in 10 μl using a multi-channel pump dispenser. Compounds were added to the protein mix by a single dip (1 nl) with a pin-transfer robot. Plates with the protein mix and added compounds were incubated on a shaker for 30 min at room temperature. The concentration of compounds during incubation was 50 μM. Reaction mixture (200 μM NADH, 150 μM DTNB in 100 mM potassium phosphate, pH 7.0, 2 mM EDTA) was added to each well in 10 μl after pre-incubation and the plate was read at 405 nm for “time 0 min” values. Plates were incubated on a shaker for 30 min at room temperature to complete the reaction and were read at 405 nm for “time 30 min.” “Time 0 min” values were subtracted from “time 30 min” values and were taken as an end-point value for the assay. Control wells contained only protein and reaction mixtures without any compounds added and were taken as 100% activity values. The final concentrations of all components during the reaction were as follows: Lpd, 100 nM; SucB, 175 nM; AhpD, 18 nM; NADH, 100 μM; DTNB, 75 μM; potassium phosphate, 50 mM; EDTA, 1 mM; compounds, 25 μM.

Table 2 lists the molecular structures of eleven chemical compounds that were identified from the above screen. The first three compounds in Table 2 were further identified as to which of the 3 enzymes (AhpD, Lpd, SucB) each inhibited.

TABLE 2
Compounds Identified From the DTNB Screening Assay

| | | | | | | | | | Macs | |
|---|---|---|---|---|---|---|---|---|---|---|
| | | | | | | | αKG- | | Via- | |
| | | | | | Kᵢ | Lpd | DH | TR | bil. | M.tb. |
| | Tem- | | IC₅₀ | | | (por- | (por- | (bo- | (50 | viabi- |
| Structure | plate | ID | (μM) | Target | (μM) | cine) | cine) | vine) | μM) | lity |
| 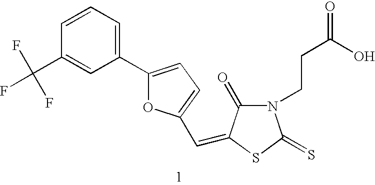 | CL- 0221 | 2556-0286 | 4.4 | SucB (Com- pet Inhib.) | 3 | NE at 10 μM | NE at 50 μM | NE at 10 μM | 80% ±1% | |
| 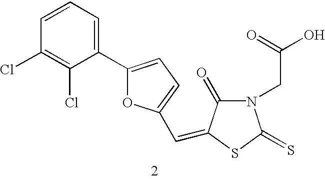 | CL- 0320 | 3229-2113 | 8.2 | SucB (Com- pet Inhib.) | 6 | NE at 10 μM | NE at 50 μM | NE at 10 μM | 91% ±5% | |
| 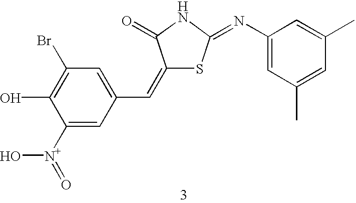 | CL- 0213 | 2368-0687 | 10.5 | AhpD (Com- pet Inhib.) | 5 | NE at 10 □M | 5-10% inhi- bition at 50 μM | NE at 10 μM | 70% ±13% *clu- sters | |
| 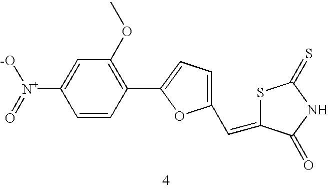 | CL- 0222 | 3269-0200 | | | | | | | | |
| 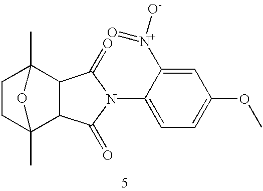 | CL- 0691 | 2360-0031 | | | | | | | | |
| 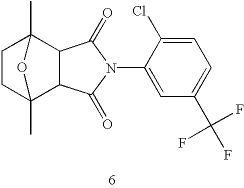 | CL- 0691 | 2360-0018 | | | | | | | | |
| 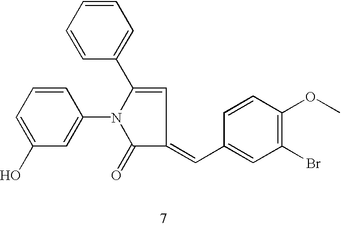 | CL- 1154 | 2150-0537 | | | | | | | | |
| 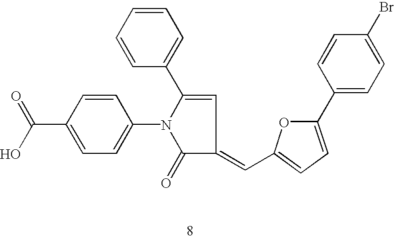 | CL- 1154 | 2150-0146 | 4.2 | | | | | | | |
| 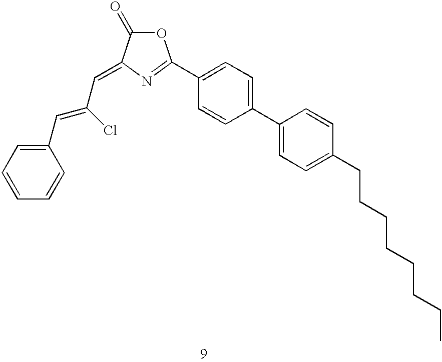 | CL- 1628 | K074-5853 | | | | | | | | |
| 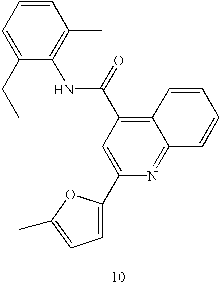 | CL- 0204 | 3366-9295 | | | | | | | | |
| 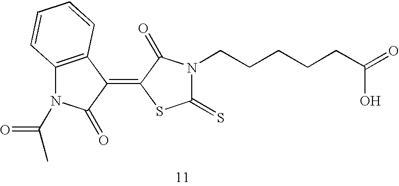 | CL- 0690 | 1503-1282 | | | | | | | | |

*“Macs Viabil.” means the viability of mouse macrophafes after approxiamately 18 hours incubation with the test compound.
*“NE” means no effect.

Although the invention has been described in detail for the purpose ration, it is understood that such detail is solely for that purpose, and ns can be made therein by those skilled in the art without departing from t and scope of the invention which is defined by the following claims.

What is claimed:

1. A method of preventing onset of tuberculosis in a subject infected with Mycobacterium tuberculosis, said method comprising:
inhibiting AhpD in the subject under conditions effective to make the pathogen susceptible to antimicrobial reactive nitrogen intermediates or reactive oxygen intermediates.

2. The method according to claim 1, wherein said inhibiting is carried out by administering an inhibitor of AhpD orally, intradermally, intramuscularly, intraperitoneally, intravenously, subcutaneously, or intranasally.

3. The method according to claim 1, wherein the AhpD is from Mycobacterium tuberculosis.

4. The method according to claim 3, wherein the AhpD is encoded by an ahpD (RV2429) gene.

5. The method according to claim 1, wherein said inhibiting is achieved with a compound which binds to one or more molecular surfaces of the AhpD having a three dimensional crystal structure defined by the atomic coordinates set forth in FIG. 1.

6. The method according to claim 5, wherein the molecular surfaces of the AhpD comprise atoms surrounding representative active site cysteine residues 130 and/or 133.

7. The method according to claim 6, wherein the molecular surface surrounding active site cysteine residue 130 is defined by a set of atomic coordinates consisting of:

| | | | | | | | |
|---|---|---|---|---|---|---|---|
| ATOM | CG | ARG | A | 86 | 26.684 | 34.263 | 9.737 |
| ATOM | CD | ARG | A | 86 | 26.287 | 34.663 | 8.311 |
| ATOM | NH1 | ARG | A | 86 | 27.197 | 34.539 | 5.647 |
| ATOM | O | ARG | A | 86 | 26.997 | 33.147 | 12.918 |
| ATOM | NE | ARG | A | 88 | 33.177 | 31.048 | 17.082 |
| ATOM | NH2 | ARG | A | 88 | 34.982 | 32.389 | 17.508 |
| ATOM | CA | GLY | A | 89 | 28.223 | 33.948 | 16.115 |
| ATOM | C | GLY | A | 89 | 26.770 | 34.038 | 16.552 |
| ATOM | O | GLY | A | 89 | 26.456 | 34.664 | 17.568 |
| ATOM | CD1 | PHE | A | 90 | 23.685 | 34.988 | 13.747 |
| ATOM | CE1 | PHE | A | 90 | 23.618 | 35.735 | 12.567 |
| ATOM | CZ | PHE | A | 90 | 23.465 | 35.086 | 11.347 |
| ATOM | CB | GLU | A | 92 | 25.004 | 34.336 | 22.064 |
| ATOM | CG | GLU | A | 92 | 23.811 | 34.962 | 21.337 |
| ATOM | CD | GLU | A | 92 | 24.154 | 36.253 | 20.615 |
| ATOM | OE1 | GLU | A | 92 | 24.690 | 37.189 | 21.252 |
| ATOM | OE2 | GLU | A | 92 | 23.877 | 36.338 | 19.400 |
| ATOM | C | GLU | A | 92 | 27.302 | 33.404 | 22.076 |
| ATOM | O | GLU | A | 92 | 27.230 | 33.531 | 23.297 |
| ATOM | N | GLY | A | 93 | 28.321 | 32.798 | 21.482 |
| ATOM | CA | GLY | A | 93 | 29.422 | 32.280 | 22.275 |
| ATOM | OD1 | ASP | A | 96 | 31.819 | 31.356 | 19.922 |
| ATOM | OD2 | ASP | A | 96 | 32.705 | 32.998 | 21.057 |
| ATOM | O | GLY | A | 129 | 27.309 | 38.037 | 7.205 |
| ATOM | SG | CYS | A | 130 | 31.238 | 35.896 | 9.779 |
| ATOM | N | SER | A | 131 | 29.608 | 39.237 | 10.219 |
| ATOM | CB | SER | A | 131 | 28.953 | 40.371 | 12.262 |
| ATOM | OG | SER | A | 131 | 29.266 | 41.435 | 13.137 |
| ATOM | N | HIS | A | 132 | 31.421 | 38.650 | 12.395 |
| ATOM | CA | HIS | A | 132 | 32.637 | 38.217 | 13.077 |
| ATOM | CB | HIS | A | 132 | 32.540 | 36.743 | 13.482 |
| ATOM | CD2 | HIS | A | 132 | 34.060 | 36.247 | 15.526 |
| ATOM | NE2 | HIS | A | 132 | 35.322 | 35.720 | 15.649 |
| ATOM | O | HIS | A | 132 | 34.836 | 39.095 | 12.675 |
| ATOM | CG1 | VAL | A | 135 | 35.077 | 43.110 | 14.983 |
| ATOM | CG2 | VAL | A | 135 | 32.949 | 43.243 | 13.686 |
| ATOM | NH1 | ARG | B | 86 | 24.434 | 40.430 | 3.551 |
| ATOM | CD1 | PHE | B | 90 | 27.146 | 43.238 | 10.807 |
| ATOM | CE1 | PHE | B | 90 | 26.195 | 42.306 | 10.382 |
| ATOM | CZ | PHE | B | 90 | 26.429 | 41.551 | 9.242 |
| ATOM | O | PHE | B | 90 | 30.581 | 45.657 | 13.145 |
| ATOM | OE2 | GLU | B | 92 | 28.060 | 46.562 | 15.789 |
| ATOM | O | GLY | B | 129 | 21.817 | 41.212 | 5.427 |

8. The method according to claim 6, wherein the molecular surface surrounding active site cysteine residue 133 is defined by a set of atomic coordinates consisting of:

| | | | | | | | |
|---|---|---|---|---|---|---|---|
| ATOM | ND2 | ASN | A | 81 | 38.756 | 31.671 | 8.422 |
| ATOM | CE1 | TYR | A | 85 | 36.018 | 31.618 | 14.046 |
| ATOM | CE2 | TYR | A | 85 | 36.646 | 31.599 | 11.723 |
| ATOM | CZ | TYR | A | 85 | 36.929 | 31.315 | 13.055 |
| ATOM | OH | TYR | A | 85 | 38.124 | 30.721 | 13.366 |
| ATOM | NH1 | ARG | A | 88 | 35.158 | 30.114 | 17.790 |
| ATOM | NH2 | ARG | A | 88 | 34.982 | 32.389 | 17.508 |
| ATOM | CB | PRO | A | 100 | 37.527 | 25.947 | 14.395 |
| ATOM | CG | PRO | A | 100 | 37.438 | 26.852 | 15.592 |
| ATOM | O | LEU | A | 102 | 41.472 | 25.358 | 10.446 |
| ATOM | N | MET | A | 104 | 43.466 | 26.552 | 7.835 |
| ATOM | CG | MET | A | 104 | 42.415 | 28.749 | 9.271 |
| ATOM | SD | MET | A | 104 | 41.163 | 29.814 | 10.015 |
| ATOM | CE | MET | A | 104 | 39.763 | 28.689 | 10.090 |
| ATOM | O | MET | A | 104 | 45.128 | 29.530 | 7.474 |
| ATOM | CA | ASN | A | 105 | 47.201 | 27.909 | 6.482 |
| ATOM | CG2 | ILE | A | 107 | 44.710 | 34.237 | 8.071 |
| ATOM | CD1 | ILE | A | 107 | 42.279 | 32.546 | 7.638 |
| ATOM | O | ILE | A | 107 | 47.536 | 34.661 | 6.821 |
| ATOM | CA | ALA | A | 108 | 49.252 | 32.809 | 7.921 |
| ATOM | CB | ALA | A | 108 | 49.613 | 31.745 | 8.959 |
| ATOM | O | ALA | A | 108 | 51.357 | 33.582 | 7.076 |
| ATOM | N | LYS | A | 114 | 50.989 | 40.121 | 4.422 |
| ATOM | CB | LYS | A | 114 | 49.659 | 39.422 | 6.349 |
| ATOM | CD | LYS | A | 114 | 50.479 | 37.681 | 7.965 |
| ATOM | CE | LYS | A | 114 | 51.122 | 36.318 | 8.106 |
| ATOM | NZ | LYS | A | 114 | 52.403 | 36.271 | 7.345 |
| ATOM | N | ALA | A | 115 | 49.121 | 42.363 | 4.988 |
| ATOM | CA | ALA | A | 115 | 48.224 | 43.514 | 4.993 |
| ATOM | CB | ALA | A | 115 | 49.021 | 44.816 | 5.065 |
| ATOM | CG | GLU | A | 118 | 45.071 | 40.454 | 7.074 |
| ATOM | OE2 | GLU | A | 118 | 44.218 | 38.267 | 7.520 |
| ATOM | CD2 | HIS | A | 132 | 34.060 | 36.247 | 15.526 |
| ATOM | CE1 | HIS | A | 132 | 35.771 | 35.402 | 14.447 |
| ATOM | NE2 | HIS | A | 132 | 35.322 | 35.720 | 15.649 |
| ATOM | O | HIS | A | 132 | 34.836 | 39.095 | 12.675 |
| ATOM | SG | CYS | A | 133 | 34.765 | 35.332 | 9.372 |
| ATOM | CG1 | VAL | A | 135 | 35.077 | 43.110 | 14.983 |
| ATOM | CA | ALA | A | 136 | 38.186 | 40.749 | 12.941 |
| ATOM | CB | ALA | A | 136 | 38.197 | 39.238 | 12.924 |
| ATOM | O | ALA | A | 136 | 40.246 | 41.806 | 12.333 |
| ATOM | ND1 | HIS | A | 137 | 40.517 | 38.915 | 8.235 |
| ATOM | CE1 | HIS | A | 137 | 40.229 | 37.629 | 8.163 |
| ATOM | NE2 | HIS | A | 137 | 38.923 | 37.502 | 8.009 |
| ATOM | CB | HIS | A | 139 | 40.410 | 44.362 | 14.769 |
| ATOM | CG | HIS | A | 139 | 41.301 | 44.548 | 15.963 |
| ATOM | CD2 | HIS | A | 139 | 42.219 | 43.729 | 16.529 |
| ATOM | CE1 | HIS | A | 139 | 42.194 | 45.593 | 17.687 |
| ATOM | NE2 | HIS | A | 139 | 42.759 | 44.403 | 17.601 |
| ATOM | OG1 | THR | A | 140 | 43.391 | 41.000 | 12.322 |
| ATOM | CG2 | THR | A | 140 | 45.224 | 41.726 | 10.943 |
| ATOM | CB | THR | A | 143 | 46.647 | 45.739 | 14.859 |
| ATOM | OG1 | THR | A | 143 | 46.606 | 44.614 | 13.978 |
| ATOM | CG2 | THR | A | 143 | 45.377 | 45.766 | 15.704 |
| ATOM | O | THR | A | 143 | 49.154 | 47.078 | 13.758 |
| ATOM | CA | VAL | A | 144 | 49.134 | 46.659 | 11.050 |
| ATOM | CB | VAL | A | 144 | 49.041 | 45.516 | 10.013 |
| ATOM | CG1 | VAL | A | 144 | 48.891 | 44.185 | 10.726 |
| ATOM | O | VAL | A | 144 | 50.259 | 48.007 | 9.412 |

9. A method of treating tuberculosis in a subject, said method comprising:
inhibiting AhpD in the subject under conditions effective to make the pathogen susceptible to antimicrobial reactive nitrogen intermediates or reactive oxygen intermediates.

10. The method according to claim 9, wherein said inhibiting is carried out by administering an inhibitor of AhpD orally, intradermally, intramuscularly, intraperitoneally, intravenously, subcutaneously, or intranasally.

11. The method according to claim 9, wherein the AhpD is from Mycobacterium tuberculosis.

12. The method according to claim 11, wherein the AhpD is encoded by an ahpD (RV2429) gene.

13. The method according to claim 9, wherein said inhibiting is achieved with a compound which binds to one or more molecular surfaces of the AhpD, having a three dimensional crystal structure defined by the atomic coordinates set forth in FIG. 1.

14. The method according to claim 13, wherein the molecular surfaces of the AhpD comprise atoms surrounding representative active site cysteine residues 130 and/or 133.

15. The method according to claim 14, wherein the molecular surface surrounding active site cysteine residue 130 is defined by a set of atomic coordinates consisting of:

| | | | | | | | |
|---|---|---|---|---|---|---|---|
| ATOM | CD | ARG | A | 86 | 26.287 | 34.663 | 8.311 |
| ATOM | NH1 | ARG | A | 86 | 27.197 | 34.539 | 5.647 |
| ATOM | O | ARG | A | 86 | 26.997 | 33.147 | 12.918 |
| ATOM | NE | ARG | A | 88 | 33.177 | 31.048 | 17.082 |
| ATOM | NH2 | ARG | A | 88 | 34.982 | 32.389 | 17.508 |
| ATOM | CA | GLY | A | 89 | 28.223 | 33.948 | 16.115 |
| ATOM | C | GLY | A | 89 | 26.770 | 34.038 | 16.552 |
| ATOM | O | GLY | A | 89 | 26.456 | 34.664 | 17.568 |
| ATOM | CD1 | PHE | A | 90 | 23.685 | 34.988 | 13.747 |
| ATOM | CE1 | PHE | A | 90 | 23.618 | 35.735 | 12.567 |
| ATOM | CZ | PHE | A | 90 | 23.465 | 35.086 | 11.347 |
| ATOM | CB | GLU | A | 92 | 25.004 | 34.336 | 22.064 |
| ATOM | CG | GLU | A | 92 | 23.811 | 34.962 | 21.337 |
| ATOM | CD | GLU | A | 92 | 24.154 | 36.253 | 20.615 |
| ATOM | OE1 | GLU | A | 92 | 24.690 | 37.189 | 21.252 |
| ATOM | OE2 | GLU | A | 92 | 23.877 | 36.338 | 19.400 |
| ATOM | C | GLU | A | 92 | 27.302 | 33.404 | 22.076 |
| ATOM | O | GLU | A | 92 | 27.230 | 33.531 | 23.297 |
| ATOM | N | GLY | A | 93 | 28.321 | 32.798 | 21.482 |
| ATOM | CA | GLY | A | 93 | 29.422 | 32.280 | 22.275 |
| ATOM | OD1 | ASP | A | 96 | 31.819 | 31.356 | 19.922 |
| ATOM | OD2 | ASP | A | 96 | 32.705 | 32.998 | 21.057 |
| ATOM | O | GLY | A | 129 | 27.309 | 38.037 | 7.205 |
| ATOM | SG | CYS | A | 130 | 31.238 | 35.896 | 9.779 |
| ATOM | N | SER | A | 131 | 29.608 | 39.237 | 10.219 |
| ATOM | CB | SER | A | 131 | 28.953 | 40.371 | 12.262 |
| ATOM | OG | SER | A | 131 | 29.266 | 41.435 | 13.137 |
| ATOM | N | HIS | A | 132 | 31.421 | 38.650 | 12.395 |
| ATOM | CA | HIS | A | 132 | 32.637 | 38.217 | 13.077 |
| ATOM | CB | HIS | A | 132 | 32.540 | 36.743 | 13.482 |
| ATOM | CD2 | HIS | A | 132 | 34.060 | 36.247 | 15.526 |
| ATOM | NE2 | HIS | A | 132 | 35.322 | 35.720 | 15.649 |
| ATOM | O | HIS | A | 132 | 34.836 | 39.095 | 12.675 |
| ATOM | OG1 | VAL | A | 135 | 35.077 | 43.110 | 14.983 |
| ATOM | CG2 | VAL | A | 135 | 32.949 | 43.243 | 13.686 |
| ATOM | NH1 | ARG | B | 86 | 24.434 | 40.430 | 3.551 |
| ATOM | CD1 | PHE | B | 90 | 27.146 | 43.238 | 10.807 |
| ATOM | CE1 | PHE | B | 90 | 26.195 | 42.306 | 10.382 |
| ATOM | CZ | PHE | B | 90 | 26.429 | 41.551 | 9.242 |
| ATOM | O | PHE | B | 90 | 30.581 | 45.657 | 13.145 |
| ATOM | OE2 | GLU | B | 92 | 28.060 | 46.562 | 15.789 |
| ATOM | O | GLY | B | 129 | 21.817 | 41.212 | 5.427 |

16. The method according to claim 14, wherein the molecular surface surrounding active site cysteine residue 133 is defined by a set of atomic coordinates consisting of:

| | | | | | | | |
|---|---|---|---|---|---|---|---|
| ATOM | ND2 | ASN | A | 81 | 38.756 | 31.671 | 8.422 |
| ATOM | CE1 | TYR | A | 85 | 36.018 | 31.618 | 14.046 |
| ATOM | CE2 | TYR | A | 85 | 36.646 | 31.599 | 11.723 |
| ATOM | CZ | TYR | A | 85 | 36.929 | 31.315 | 13.055 |
| ATOM | OH | TYR | A | 85 | 38.124 | 30.721 | 13.366 |
| ATOM | NH1 | ARG | A | 88 | 35.158 | 30.114 | 17.790 |
| ATOM | NH2 | ARG | A | 88 | 34.982 | 32.389 | 17.508 |
| ATOM | CB | PRO | A | 100 | 37.527 | 25.947 | 14.395 |
| ATOM | CG | PRO | A | 100 | 37.438 | 26.852 | 15.592 |
| ATOM | O | LEU | A | 102 | 41.472 | 25.358 | 10.446 |
| ATOM | N | MET | A | 104 | 43.466 | 26.552 | 7.835 |
| ATOM | CG | MET | A | 104 | 42.415 | 28.749 | 9.271 |
| ATOM | SD | MET | A | 104 | 41.163 | 29.814 | 10.015 |
| ATOM | CE | MET | A | 104 | 39.763 | 28.689 | 10.090 |
| ATOM | O | MET | A | 104 | 45.128 | 29.530 | 7.474 |
| ATOM | CA | ASN | A | 105 | 47.201 | 27.909 | 6.482 |
| ATOM | CG2 | ILE | A | 107 | 44.710 | 34.237 | 8.071 |
| ATOM | CD1 | ILE | A | 107 | 42.279 | 32.546 | 7.638 |
| ATOM | O | ILE | A | 107 | 47.536 | 34.661 | 6.821 |
| ATOM | CA | ALA | A | 108 | 49.252 | 32.809 | 7.921 |
| ATOM | CB | ALA | A | 108 | 49.613 | 31.745 | 8.959 |
| ATOM | O | ALA | A | 108 | 51.357 | 33.582 | 7.076 |
| ATOM | N | LYS | A | 114 | 50.989 | 40.121 | 4.422 |
| ATOM | CB | LYS | A | 114 | 49.659 | 39.422 | 6.349 |
| ATOM | CD | LYS | A | 114 | 50.479 | 37.681 | 7.965 |
| ATOM | CE | LYS | A | 114 | 51.122 | 36.318 | 8.106 |
| ATOM | NZ | LYS | A | 114 | 52.403 | 36.271 | 7.345 |
| ATOM | N | ALA | A | 115 | 49.121 | 42.363 | 4.988 |
| ATOM | CA | ALA | A | 115 | 48.224 | 43.514 | 4.993 |
| ATOM | CB | ALA | A | 115 | 49.021 | 44.816 | 5.065 |
| ATOM | CG | GLU | A | 118 | 45.071 | 40.454 | 7.074 |
| ATOM | OE2 | GLU | A | 118 | 44.218 | 38.267 | 7.520 |
| ATOM | CD2 | HIS | A | 132 | 34.060 | 36.247 | 15.526 |
| ATOM | CE1 | HIS | A | 132 | 35.771 | 35.402 | 14.447 |
| ATOM | NE2 | HIS | A | 132 | 35.322 | 35.720 | 15.649 |
| ATOM | O | HIS | A | 132 | 34.836 | 39.095 | 12.675 |
| ATOM | SG | CYS | A | 133 | 34.765 | 35.332 | 9.372 |
| ATOM | CG1 | VAL | A | 135 | 35.077 | 43.110 | 14.983 |
| ATOM | CA | ALA | A | 136 | 38.186 | 40.749 | 12.941 |
| ATOM | CB | ALA | A | 136 | 38.197 | 39.238 | 12.924 |
| ATOM | O | ALA | A | 136 | 40.246 | 41.806 | 12.333 |
| ATOM | ND1 | HIS | A | 137 | 40.517 | 38.915 | 8.235 |
| ATOM | CE1 | HIS | A | 137 | 40.229 | 37.629 | 8.163 |
| ATOM | NE2 | HIS | A | 137 | 38.923 | 37.502 | 8.009 |
| ATOM | CB | HIS | A | 139 | 40.410 | 44.362 | 14.769 |
| ATOM | CG | HIS | A | 139 | 41.301 | 44.548 | 15.963 |
| ATOM | CD2 | HIS | A | 139 | 42.219 | 43.729 | 16.529 |
| ATOM | CE1 | HIS | A | 139 | 42.194 | 45.593 | 17.687 |
| ATOM | NE2 | HIS | A | 139 | 42.759 | 44.403 | 17.601 |
| ATOM | OG1 | THR | A | 140 | 43.391 | 41.000 | 12.322 |
| ATOM | CG2 | THR | A | 140 | 45.224 | 41.726 | 10.943 |
| ATOM | CB | THR | A | 143 | 46.647 | 45.739 | 14.859 |
| ATOM | OG1 | THR | A | 143 | 46.606 | 44.614 | 13.978 |
| ATOM | CG2 | THR | A | 143 | 45.377 | 45.766 | 15.704 |
| ATOM | O | THR | A | 143 | 49.154 | 47.078 | 13.758 |
| ATOM | CA | VAL | A | 144 | 49.134 | 46.659 | 11.050 |
| ATOM | CB | VAL | A | 144 | 49.041 | 45.516 | 10.013 |
| ATOM | CG1 | VAL | A | 144 | 48.891 | 44.185 | 10.726 |
| ATOM | O | VAL | A | 144 | 50.259 | 48.007 | 9.412 |

17. A method of preventing onset of tuberculosis in a subject infected with Mycobacterium tuberculosis, said method comprising:
inhibiting dihydrolipoamide dehydrogenase in Mycobacterium tuberculosis in the subject under conditions effective to make the pathogen susceptible to antimicrobial reactive nitrogen intermediates or reactive oxygen intermediates.

18. The method according to claim 17, wherein said inhibiting is carried out by administering an inhibitor of dihydrolipoamide dehydrogenase orally, intradermally, intramuscularly, intraperitoneally, intravenously, subcutaneously, or intranasally.

19. The method according to claim 17, wherein the dihydrolipoamide dehydrogenase is encoded by an RV0462 gene.

20. A method of treating tuberculosis in a subject, said method comprising:
inhibiting dihydrolipoamide dehydrogenase in Mycobacterium tuberculosis in the subject under conditions effective to make the pathogen susceptible to antimicrobial reactive nitrogen intermediates or reactive oxygen intermediates.

21. The method according to claim 20, wherein said inhibiting is carried out by administering an inhibitor of dihydrolipoamide dehydrogenase orally, intradermally, intramuscularly, intraperitoneally, intravenously, subcutaneously, or intranasally.

22. The method according to claim 23, wherein the dihydrolipoamide dehydrogenase is encoded by an RV0462 gene.

23. A method of preventing onset of tuberculosis in a subject infected with Mycobacterium tuberculosis, said method comprising:
inhibiting dihydrolipoamide succinyltransferase in Mycobacterium tuberculosis in the subject under conditions effective to make the pathogen susceptible to antimicrobial reactive nitrogen intermediates or reactive oxygen intermediates.

24. The method according to claim 23, wherein said inhibiting is carried out by administering an inhibitor of dihydrolipoamide succinyltransferase orally, intradermally, intramuscularly, intraperitoneally, intravenously, subcutaneously, or intranasally.

25. The method according to claim 23, wherein the dihydrolipoamide succinyltransferase is encoded by a sucB (RV2215) gene.

26. A method of treating tuberculosis in a subject, said method comprising:
inhibiting dihydrolipoamide succinyltransferase in Mycobacterium tuberculosis in the subject under conditions effective to make the pathogen susceptible to antimicrobial reactive nitrogen intermediates or reactive oxygen intermediates.

27. The method according to claim 26, wherein said inhibiting is carried out by administering an inhibitor of dihydrolipoamide succinyltransferase orally, intraderrnally, intramuscularly, intraperitoneally, intravenously, subcutaneously, or intranasally.

28. The method according to claim 26, wherein the dihydrolipoamide succinyltransferase is encoded by a sucB (RV2215) gene.

29. A method of producing an AhpD crystal suitable for X-ray diffraction comprising:
subjecting a solution of AhpD under conditions effective to grow a crystal of AhpD to a size suitable for X-ray diffraction; and
obtaining an AhpD crystal suitable for X-ray diffraction.

30. The method of claim 29, wherein the crystal has space group P6₅22 and unit cell dimensions of approximately a=108.3 Å, b=108.3 Å, and c=233.6 Å such that the three dimensional structure of the crystallized AhpD can be determined to a resolution of about 2.0 Å or better.

31. The method of claim 29, wherein crystallization occurs in hanging drops using a vapor diffusion method.

32. A crystal produced by the method of claim 29.

33. A method for identifying candidate compounds suitable for treatment or prevention of tuberculosis in a subject, said method comprising:
contacting AhpD with a compound and
identifying those compounds which bind to the AhpD as candidate compounds suitable for treatment or prevention of tuberculosis in a subject.

34. The method according to claim 33, wherein the AhpD is from Mycobacterium tuberculosis.

35. The method according to claim 34, wherein the AhpD is encoded by an ahpD (RV2429) gene.

36. The method according to claim 33, wherein the compound binds to one or more molecular surfaces of the AhpD, having a three dimensional crystal structure defined by the atomic coordinates set forth in FIG. 1.

37. The method according to claim 36, wherein the molecular surfaces of the AhpD comprise atoms surrounding representative active site cysteine residues 130 and/or 133.

38. The method according to claim 37, wherein the representative molecular surface surrounding active site cysteine residue 130 is defined by a set of atomic coordinates consisting of:

| | | | | | | | |
|---|---|---|---|---|---|---|---|
| ATOM | CG | ARG | A | 86 | 26.684 | 34.263 | 9.737 |
| ATOM | CD | ARG | A | 86 | 26.287 | 34.663 | 8.311 |
| ATOM | NH1 | ARG | A | 86 | 27.197 | 34.539 | 5.647 |
| ATOM | O | ARG | A | 86 | 26.997 | 33.147 | 12.918 |
| ATOM | NE | ARG | A | 88 | 33.177 | 31.048 | 17.082 |
| ATOM | NH2 | ARG | A | 88 | 34.982 | 32.389 | 17.508 |
| ATOM | CA | GLY | A | 89 | 28.223 | 33.948 | 16.115 |
| ATOM | C | GLY | A | 89 | 26.770 | 34.038 | 16.552 |
| ATOM | O | GLY | A | 89 | 26.456 | 34.664 | 17.568 |
| ATOM | CD1 | PHE | A | 90 | 23.685 | 34.988 | 13.747 |
| ATOM | CE1 | PHE | A | 90 | 23.618 | 35.735 | 12.567 |
| ATOM | CZ | PHE | A | 90 | 23.465 | 35.086 | 11.347 |
| ATOM | CB | GLU | A | 92 | 25.004 | 34.336 | 22.064 |
| ATOM | CG | GLU | A | 92 | 23.811 | 34.962 | 21.337 |
| ATOM | CD | GLU | A | 92 | 24.154 | 36.253 | 20.615 |
| ATOM | OE1 | GLU | A | 92 | 24.690 | 37.189 | 21.252 |
| ATOM | OE2 | GLU | A | 92 | 23.877 | 36.338 | 19.400 |
| ATOM | C | GLU | A | 92 | 27.302 | 33.404 | 22.076 |
| ATOM | O | GLU | A | 92 | 27.230 | 33.531 | 23.297 |
| ATOM | N | GLY | A | 93 | 28.321 | 32.798 | 21.482 |
| ATOM | CA | GLY | A | 93 | 29.422 | 32.280 | 22.275 |
| ATOM | OD1 | ASP | A | 96 | 31.819 | 31.356 | 19.922 |
| ATOM | OD2 | ASP | A | 96 | 32.705 | 32.998 | 21.057 |
| ATOM | O | GLY | A | 129 | 27.309 | 38.037 | 7.205 |
| ATOM | SG | CYS | A | 130 | 31.238 | 35.896 | 9.779 |
| ATOM | N | SER | A | 131 | 29.608 | 39.237 | 10.219 |
| ATOM | CB | SER | A | 131 | 28.953 | 40.371 | 12.262 |
| ATOM | OG | SER | A | 131 | 29.266 | 41.435 | 13.137 |
| ATOM | N | HIS | A | 132 | 31.421 | 38.650 | 12.395 |
| ATOM | CA | HIS | A | 132 | 32.637 | 38.217 | 13.077 |
| ATOM | CB | HIS | A | 132 | 32.540 | 36.743 | 13.482 |
| ATOM | CD2 | HIS | A | 132 | 34.060 | 36.247 | 15.526 |
| ATOM | NE2 | HIS | A | 132 | 35.322 | 35.720 | 15.649 |
| ATOM | O | HIS | A | 132 | 34.836 | 39.095 | 12.675 |
| ATOM | CG1 | VAL | A | 135 | 35.077 | 43.110 | 14.983 |
| ATOM | CG2 | VAL | A | 135 | 32.949 | 43.243 | 13.686 |
| ATOM | NH1 | ARG | B | 86 | 24.434 | 40.430 | 3.551 |
| ATOM | CD1 | PHE | B | 90 | 27.146 | 43.238 | 10.807 |
| ATOM | CE1 | PHE | B | 90 | 26.195 | 42.306 | 10.382 |
| ATOM | CZ | PHE | B | 90 | 26.429 | 41.551 | 9.242 |
| ATOM | O | PHE | B | 90 | 30.581 | 45.657 | 13.145 |
| ATOM | OE2 | GLU | B | 92 | 28.060 | 46.562 | 15.789 |
| ATOM | O | GLY | B | 129 | 21.817 | 41.212 | 5.427 |

39. The method according to claim 37, wherein the molecular surface surrounding active site cysteine residue 133 is defined by a set of atomic coordinates consisting of:

| | | | | | | | |
|---|---|---|---|---|---|---|---|
| ATOM | ND2 | ASN | A | 81 | 38.756 | 31.671 | 8.422 |
| ATOM | CE1 | TYR | A | 85 | 36.018 | 31.618 | 14.046 |
| ATOM | CE2 | TYR | A | 85 | 36.646 | 31.599 | 11.723 |
| ATOM | CZ | TYR | A | 85 | 36.929 | 31.315 | 13.055 |
| ATOM | OH | TYR | A | 85 | 38.124 | 30.721 | 13.366 |
| ATOM | NH1 | ARG | A | 88 | 35.158 | 30.114 | 17.790 |
| ATOM | NH2 | ARG | A | 88 | 34.982 | 32.389 | 17.508 |
| ATOM | CB | PRO | A | 100 | 37.527 | 25.947 | 14.395 |
| ATOM | CG | PRO | A | 100 | 37.438 | 26.852 | 15.592 |
| ATOM | O | LEU | A | 102 | 41.472 | 25.358 | 10.446 |
| ATOM | N | MET | A | 104 | 43.466 | 26.552 | 7.835 |
| ATOM | CG | MET | A | 104 | 42.415 | 28.749 | 9.271 |
| ATOM | SD | MET | A | 104 | 41.163 | 29.814 | 10.015 |
| ATOM | CE | MET | A | 104 | 39.763 | 28.689 | 10.090 |
| ATOM | O | MET | A | 104 | 45.128 | 29.530 | 7.474 |
| ATOM | CA | ASN | A | 105 | 47.201 | 27.909 | 6.482 |
| ATOM | CG2 | ILE | A | 107 | 44.710 | 34.237 | 8.071 |
| ATOM | CD1 | ILE | A | 107 | 42.279 | 32.546 | 7.638 |
| ATOM | O | ILE | A | 107 | 47.536 | 34.661 | 6.821 |
| ATOM | CA | ALA | A | 108 | 49.252 | 32.809 | 7.921 |
| ATOM | CB | ALA | A | 108 | 49.613 | 31.745 | 8.959 |
| ATOM | O | ALA | A | 108 | 51.357 | 33.582 | 7.076 |
| ATOM | N | LYS | A | 114 | 50.989 | 40.121 | 4.422 |
| ATOM | CB | LYS | A | 114 | 49.659 | 39.422 | 6.349 |
| ATOM | CD | LYS | A | 114 | 50.479 | 37.681 | 7.965 |
| ATOM | CE | LYS | A | 114 | 51.122 | 36.318 | 8.106 |
| ATOM | NZ | LYS | A | 114 | 52.403 | 36.271 | 7.345 |
| ATOM | N | ALA | A | 115 | 49.121 | 42.363 | 4.988 |
| ATOM | CA | ALA | A | 115 | 48.224 | 43.514 | 4.993 |
| ATOM | CB | ALA | A | 115 | 49.021 | 44.816 | 5.065 |
| ATOM | CG | GLU | A | 118 | 45.071 | 40.454 | 7.074 |
| ATOM | OE2 | GLU | A | 118 | 44.218 | 38.267 | 7.520 |
| ATOM | CD2 | HIS | A | 132 | 34.060 | 36.247 | 15.526 |
| ATOM | CE1 | HIS | A | 132 | 35.771 | 35.402 | 14.447 |
| ATOM | NE2 | HIS | A | 132 | 35.322 | 35.720 | 15.649 |
| ATOM | O | HIS | A | 132 | 34.836 | 39.095 | 12.675 |
| ATOM | SG | CYS | A | 133 | 34.765 | 35.332 | 9.372 |
| ATOM | CG1 | VAL | A | 135 | 35.077 | 43.110 | 14.983 |
| ATOM | CA | ALA | A | 136 | 38.186 | 40.749 | 12.941 |
| ATOM | CB | ALA | A | 136 | 38.197 | 39.238 | 12.924 |
| ATOM | O | ALA | A | 136 | 40.246 | 41.806 | 12.333 |
| ATOM | ND1 | HIS | A | 137 | 40.517 | 38.915 | 8.235 |
| ATOM | CE1 | HIS | A | 137 | 40.229 | 37.629 | 8.163 |
| ATOM | NE2 | HIS | A | 137 | 38.923 | 37.502 | 8.009 |
| ATOM | CB | HIS | A | 139 | 40.410 | 44.362 | 14.769 |
| ATOM | CG | HIS | A | 139 | 41.301 | 44.548 | 15.963 |
| ATOM | CD2 | HIS | A | 139 | 42.219 | 43.729 | 16.529 |
| ATOM | CE1 | HIS | A | 139 | 42.194 | 45.593 | 17.687 |
| ATOM | NE2 | HIS | A | 139 | 42.759 | 44.403 | 17.601 |
| ATOM | OG1 | THR | A | 140 | 43.391 | 41.000 | 12.322 |
| ATOM | CG2 | THR | A | 140 | 45.224 | 41.726 | 10.943 |
| ATOM | CB | THR | A | 143 | 46.647 | 45.739 | 14.859 |
| ATOM | OG1 | THR | A | 143 | 46.606 | 44.614 | 13.978 |
| ATOM | CG2 | THR | A | 143 | 45.377 | 45.766 | 15.704 |
| ATOM | O | THR | A | 143 | 49.154 | 47.078 | 13.758 |
| ATOM | CA | VAL | A | 144 | 49.134 | 46.659 | 11.050 |
| ATOM | CB | VAL | A | 144 | 49.041 | 45.516 | 10.013 |
| ATOM | CG1 | VAL | A | 144 | 48.891 | 44.185 | 10.726 |
| ATOM | O | VAL | A | 144 | 50.259 | 48.007 | 9.412 |

40. A method for identifying candidate compounds suitable for treatment or prevention of tuberculosis in a subject, said method comprising:
contacting a dihydrolipoamide dehydrogenase in Mycobacterium tuberculosis with a compound and
identifying those compounds which bind to the dihydrolipoamide dehydrogenase as candidate compounds suitable for treatment or prevention of tuberculosis in a subject.

41. The method according to claim 40, wherein the dihydrolipoamide dehydrogenase is encoded by an RV0462 gene.

42. A method for identifying candidate compounds suitable for treatment or prevention of tuberculosis in a subject, said method comprising:
contacting a dihydrolipoamide succinyltransferase in Mycobacterium tuberculosis with a compound and
identifying those compounds which bind to the dihydrolipoamide succinyltransferase as candidate compounds suitable for treatment or prevention of pathogen infection in a subject.

43. The method according to claim 42, wherein the dihydrolipoamide succinyltransferase is encoded by a sucB (RV22 15) gene.

44. A method for designing a compound suitable for treatment or prevention of tuberculosis in a subject, said method comprising:
providing a three-dimensional structure of a crystallized AhpD; and
designing a compound having a three-dimensional structure which will bind to one or more molecular surfaces of the AhpD.

45. The method according to claim 44, wherein the AhpD is from Mycobacterium tuberculosis.

46. The method according to claim 44, wherein the three dimensional structure of a crystallized AhpD is defined by the atomic coordinates set forth in FIG. 1.

47. The method according to claim 46, wherein the molecular surfaces of the AhpD comprise atoms surrounding representative active site cysteine residues 130 and/or 133.

48. The method according to claim 47, wherein the molecular surface surrounding active site cysteine residue 130 is defined by a set of atomic coordinates consisting of:

| | | | | | | | |
|---|---|---|---|---|---|---|---|
| ATOM | CG | ARG | A | 86 | 26.684 | 34.263 | 9.737 |
| ATOM | CD | ARG | A | 86 | 26.287 | 34.663 | 8.311 |
| ATOM | NH1 | ARG | A | 86 | 27.197 | 34.539 | 5.647 |
| ATOM | O | ARG | A | 86 | 26.997 | 33.147 | 12.918 |
| ATOM | NE | ARG | A | 88 | 33.177 | 31.048 | 17.082 |
| ATOM | NH2 | ARG | A | 88 | 34.982 | 32.389 | 17.508 |
| ATOM | CA | GLY | A | 89 | 28.223 | 33.948 | 16.115 |
| ATOM | C | GLY | A | 89 | 26.770 | 34.038 | 16.552 |
| ATOM | O | GLY | A | 89 | 26.456 | 34.664 | 17.568 |
| ATOM | CD1 | PHE | A | 90 | 23.685 | 34.988 | 13.747 |
| ATOM | CE1 | PHE | A | 90 | 23.618 | 35.735 | 12.567 |
| ATOM | CZ | PHE | A | 90 | 23.465 | 35.086 | 11.347 |
| ATOM | CB | GLU | A | 92 | 25.004 | 34.336 | 22.064 |
| ATOM | CG | GLU | A | 92 | 23.811 | 34.962 | 21.337 |
| ATOM | CD | GLU | A | 92 | 24.154 | 36.253 | 20.615 |
| ATOM | OE1 | GLU | A | 92 | 24.690 | 37.189 | 21.252 |
| ATOM | OE2 | GLU | A | 92 | 23.877 | 36.338 | 19.400 |
| ATOM | C | GLU | A | 92 | 27.302 | 33.404 | 22.076 |
| ATOM | O | GLU | A | 92 | 27.230 | 33.531 | 23.297 |
| ATOM | N | GLY | A | 93 | 28.321 | 32.798 | 21.482 |
| ATOM | CA | GLY | A | 93 | 29.422 | 32.280 | 22.275 |
| ATOM | OD1 | ASP | A | 96 | 31.819 | 31.356 | 19.922 |
| ATOM | OD2 | ASP | A | 96 | 32.705 | 32.998 | 21.057 |
| ATOM | O | GLY | A | 129 | 27.309 | 38.037 | 7.205 |
| ATOM | SG | CYS | A | 130 | 31.238 | 35.896 | 9.779 |
| ATOM | N | SER | A | 131 | 29.608 | 39.237 | 10.219 |
| ATOM | CB | SER | A | 131 | 28.953 | 40.371 | 12.262 |
| ATOM | OG | SER | A | 131 | 29.266 | 41.435 | 13.137 |
| ATOM | N | HIS | A | 132 | 31.421 | 38.650 | 12.395 |
| ATOM | CA | HIS | A | 132 | 32.637 | 38.217 | 13.077 |
| ATOM | CB | HIS | A | 132 | 32.540 | 36.743 | 13.482 |
| ATOM | CD2 | HIS | A | 132 | 34.060 | 36.247 | 15.526 |
| ATOM | NE2 | HIS | A | 132 | 35.322 | 35.720 | 15.649 |
| ATOM | O | HIS | A | 132 | 34.836 | 39.095 | 12.675 |
| ATOM | CG1 | VAL | A | 135 | 35.077 | 43.110 | 14.983 |
| ATOM | CG2 | VAL | A | 135 | 32.949 | 43.243 | 13.686 |
| ATOM | NH1 | ARG | B | 86 | 24.434 | 40.430 | 3.551 |
| ATOM | CD1 | PHE | B | 90 | 27.146 | 43.238 | 10.807 |
| ATOM | CE1 | PHE | B | 90 | 26.195 | 42.306 | 10.382 |
| ATOM | CZ | PHE | B | 90 | 26.429 | 41.551 | 9.242 |
| ATOM | O | PHE | B | 90 | 30.581 | 45.657 | 13.145 |
| ATOM | OE2 | GLU | B | 92 | 28.060 | 46.562 | 15.789 |
| ATOM | O | GLY | B | 129 | 21.817 | 41.212 | 5.427 |

49. The method according to claim 47, wherein the molecular surface surrounding active site cysteine residue 133 is defined by a set of atomic coordinates consisting of:

| | | | | | | | |
|---|---|---|---|---|---|---|---|
| ATOM | ND2 | ASN | A | 81 | 38.756 | 31.671 | 8.422 |
| ATOM | CE1 | TYR | A | 85 | 36.018 | 31.618 | 14.046 |
| ATOM | CE2 | TYR | A | 85 | 36.646 | 31.599 | 11.723 |
| ATOM | CZ | TYR | A | 85 | 36.929 | 31.315 | 13.055 |
| ATOM | OH | TYR | A | 85 | 38.124 | 30.721 | 13.366 |
| ATOM | NH1 | ARG | A | 88 | 35.158 | 30.114 | 17.790 |
| ATOM | NH2 | ARG | A | 88 | 34.982 | 32.389 | 17.508 |
| ATOM | CB | PRO | A | 100 | 37.527 | 25.947 | 14.395 |
| ATOM | CG | PRO | A | 100 | 37.438 | 26.852 | 15.592 |
| ATOM | O | LEU | A | 102 | 41.472 | 25.358 | 10.446 |
| ATOM | N | MET | A | 104 | 43.466 | 26.552 | 7.835 |
| ATOM | CG | MET | A | 104 | 42.415 | 28.749 | 9.271 |
| ATOM | SD | MET | A | 104 | 41.163 | 29.814 | 10.015 |
| ATOM | CE | MET | A | 104 | 39.763 | 28.689 | 10.090 |
| ATOM | O | MET | A | 104 | 45.128 | 29.530 | 7.474 |
| ATOM | CA | ASN | A | 105 | 47.201 | 27.909 | 6.482 |
| ATOM | CG2 | ILE | A | 107 | 44.710 | 34.237 | 8.071 |
| ATOM | CD1 | ILE | A | 107 | 42.279 | 32.546 | 7.638 |
| ATOM | O | ILE | A | 107 | 47.536 | 34.661 | 6.821 |
| ATOM | CA | ALA | A | 108 | 49.252 | 32.809 | 7.921 |
| ATOM | CB | ALA | A | 108 | 49.613 | 31.745 | 8.959 |
| ATOM | O | ALA | A | 108 | 51.357 | 33.582 | 7.076 |
| ATOM | N | LYS | A | 114 | 50.989 | 40.121 | 4.422 |
| ATOM | CB | LYS | A | 114 | 49.659 | 39.422 | 6.349 |
| ATOM | CD | LYS | A | 114 | 50.479 | 37.681 | 7.965 |
| ATOM | CE | LYS | A | 114 | 51.122 | 36.318 | 8.106 |
| ATOM | NZ | LYS | A | 114 | 52.403 | 36.271 | 7.345 |
| ATOM | N | ALA | A | 115 | 49.121 | 42.363 | 4.988 |
| ATOM | CA | ALA | A | 115 | 48.224 | 43.514 | 4.993 |
| ATOM | CB | ALA | A | 115 | 49.021 | 44.816 | 5.065 |
| ATOM | CG | GLU | A | 118 | 45.071 | 40.454 | 7.074 |
| ATOM | OE2 | GLU | A | 118 | 44.218 | 38.267 | 7.520 |
| ATOM | CD2 | HIS | A | 132 | 34.060 | 36.247 | 15.526 |
| ATOM | CE1 | HIS | A | 132 | 35.771 | 35.402 | 14.447 |
| ATOM | NE2 | HIS | A | 132 | 35.322 | 35.720 | 15.649 |
| ATOM | O | HIS | A | 132 | 34.836 | 39.095 | 12.675 |
| ATOM | SG | CYS | A | 133 | 34.765 | 35.332 | 9.372 |
| ATOM | CG1 | VAL | A | 135 | 35.077 | 43.110 | 14.983 |
| ATOM | CA | ALA | A | 136 | 38.186 | 40.749 | 12.941 |
| ATOM | CB | ALA | A | 136 | 38.197 | 39.238 | 12.924 |
| ATOM | O | ALA | A | 136 | 40.246 | 41.806 | 12.333 |
| ATOM | ND1 | HIS | A | 137 | 40.517 | 38.915 | 8.235 |
| ATOM | CE1 | HIS | A | 137 | 40.229 | 37.629 | 8.163 |
| ATOM | NE2 | HIS | A | 137 | 38.923 | 37.502 | 8.009 |
| ATOM | CB | HIS | A | 139 | 40.410 | 44.362 | 14.769 |
| ATOM | CG | HIS | A | 139 | 41.301 | 44.548 | 15.963 |
| ATOM | CD2 | HIS | A | 139 | 42.219 | 43.729 | 16.529 |
| ATOM | CE1 | HIS | A | 139 | 42.194 | 45.593 | 17.687 |
| ATOM | NE2 | HIS | A | 139 | 42.759 | 44.403 | 17.601 |
| ATOM | OG1 | THR | A | 140 | 43.391 | 41.000 | 12.322 |
| ATOM | CG2 | THR | A | 140 | 45.224 | 41.726 | 10.943 |
| ATOM | CB | THR | A | 143 | 46.647 | 45.739 | 14.859 |
| ATOM | OG1 | THR | A | 143 | 46.606 | 44.614 | 13.978 |
| ATOM | CG2 | THR | A | 143 | 45.377 | 45.766 | 15.704 |
| ATOM | O | THR | A | 143 | 49.154 | 47.078 | 13.758 |
| ATOM | CA | VAL | A | 144 | 49.134 | 46.659 | 11.050 |
| ATOM | CB | VAL | A | 144 | 49.041 | 45.516 | 10.013 |
| ATOM | CG1 | VAL | A | 144 | 48.891 | 44.185 | 10.726 |
| ATOM | O | VAL | A | 144 | 50.259 | 48.007 | 9.412 |

50. A compound designed by the method of claim 44.

51. A pharmaceutical composition comprising the compound of claim 50 and a pharmaceutical carrier.

52. A compound suitable for treatment or prevention of tuberculosis in a subject, said compound having a three-dimensional structure which will bind to one or more molecular surfaces of the AhpD having a three dimensional crystal structure defined by the atomic coordinates set forth in FIG. 1.

53. The compound according to claim 52, wherein the molecular surfaces of the AhpD comprise atoms surrounding representative active site cysteine residues 130 and/or 133.

54. The compound according to claim 53, wherein the molecular surface surrounding active site cysteine residue 130 is defined by a set of atomic coordinates consisting of:

| | | | | | | | |
|---|---|---|---|---|---|---|---|
| ATOM | CG | ARG | A | 86 | 26.684 | 34.263 | 9.737 |
| ATOM | CD | ARG | A | 86 | 26.287 | 34.663 | 8.311 |
| ATOM | NH1 | ARG | A | 86 | 27.197 | 34.539 | 5.647 |
| ATOM | O | ARG | A | 86 | 26.997 | 33.147 | 12.918 |
| ATOM | NE | ARG | A | 88 | 33.177 | 31.048 | 17.082 |
| ATOM | NH2 | ARG | A | 88 | 34.982 | 32.389 | 17.508 |
| ATOM | CA | GLY | A | 89 | 28.223 | 33.948 | 16.115 |
| ATOM | C | GLY | A | 89 | 26.770 | 34.038 | 16.552 |
| ATOM | O | GLY | A | 89 | 26.456 | 34.664 | 17.568 |
| ATOM | CD1 | PHE | A | 90 | 23.685 | 34.988 | 13.747 |
| ATOM | CE1 | PHE | A | 90 | 23.618 | 35.735 | 12.567 |
| ATOM | CZ | PHE | A | 90 | 23.465 | 35.086 | 11.347 |
| ATOM | CB | GLU | A | 92 | 25.004 | 34.336 | 22.064 |
| ATOM | CG | GLU | A | 92 | 23.811 | 34.962 | 21.337 |
| ATOM | CD | GLU | A | 92 | 24.154 | 36.253 | 20.615 |
| ATOM | OE1 | GLU | A | 92 | 24.690 | 37.189 | 21.252 |
| ATOM | OE2 | GLU | A | 92 | 23.877 | 36.338 | 19.400 |
| ATOM | C | GLU | A | 92 | 27.302 | 33.404 | 22.076 |
| ATOM | O | GLU | A | 92 | 27.230 | 33.531 | 23.297 |
| ATOM | N | GLY | A | 93 | 28.321 | 32.798 | 21.482 |
| ATOM | CA | GLY | A | 93 | 29.422 | 32.280 | 22.275 |
| ATOM | OD1 | ASP | A | 96 | 31.819 | 31.356 | 19.922 |
| ATOM | OD2 | ASP | A | 96 | 32.705 | 32.998 | 21.057 |
| ATOM | O | GLY | A | 129 | 27.309 | 38.037 | 7.205 |
| ATOM | SG | CYS | A | 130 | 31.238 | 35.896 | 9.779 |
| ATOM | N | SER | A | 131 | 29.608 | 39.237 | 10.219 |
| ATOM | CB | SER | A | 131 | 28.953 | 40.371 | 12.262 |
| ATOM | OG | SER | A | 131 | 29.266 | 41.435 | 13.137 |
| ATOM | N | HIS | A | 132 | 31.421 | 38.650 | 12.395 |
| ATOM | CA | HIS | A | 132 | 32.637 | 38.217 | 13.077 |
| ATOM | CB | HIS | A | 132 | 32.540 | 36.743 | 13.482 |
| ATOM | CD2 | HIS | A | 132 | 34.060 | 36.247 | 15.526 |
| ATOM | NE2 | HIS | A | 132 | 35.322 | 35.720 | 15.649 |
| ATOM | O | HIS | A | 132 | 34.836 | 39.095 | 12.675 |
| ATOM | CG1 | VAL | A | 135 | 35.077 | 43.110 | 14.983 |
| ATOM | CG2 | VAL | A | 135 | 32.949 | 43.243 | 13.686 |
| ATOM | NH1 | ARG | B | 86 | 24.434 | 40.430 | 3.551 |
| ATOM | CD1 | PHE | B | 90 | 27.146 | 43.238 | 10.807 |
| ATOM | CE1 | PHE | B | 90 | 26.195 | 42.306 | 10.382 |
| ATOM | CZ | PHE | B | 90 | 26.429 | 41.551 | 9.242 |
| ATOM | O | PHE | B | 90 | 30.581 | 45.657 | 13.145 |
| ATOM | OE2 | GLU | B | 92 | 28.060 | 46.562 | 15.789 |
| ATOM | O | GLY | B | 129 | 21.817 | 41.212 | 5.427 |

55. The compound according to claim 53, wherein the molecular surface surrounding active site cysteine residue 133 is defined by a set of atomic coordinates consisting of:

| | | | | | | | |
|---|---|---|---|---|---|---|---|
| ATOM | ND2 | ASN | A | 81 | 38.756 | 31.671 | 8.422 |
| ATOM | CE1 | TYR | A | 85 | 36.018 | 31.618 | 14.046 |
| ATOM | CE2 | TYR | A | 85 | 36.646 | 31.599 | 11.723 |
| ATOM | CZ | TYR | A | 85 | 36.929 | 31.315 | 13.055 |
| ATOM | OH | TYR | A | 85 | 38.124 | 30.721 | 13.366 |
| ATOM | NH1 | ARG | A | 88 | 35.158 | 30.114 | 17.790 |
| ATOM | NH2 | ARG | A | 88 | 34.982 | 32.389 | 17.508 |
| ATOM | CB | PRO | A | 100 | 37.527 | 25.947 | 14.395 |
| ATOM | CG | PRO | A | 100 | 37.438 | 26.852 | 15.592 |
| ATOM | O | LEU | A | 102 | 41.472 | 25.358 | 10.446 |
| ATOM | N | MET | A | 104 | 43.466 | 26.552 | 7.835 |
| ATOM | CG | MET | A | 104 | 42.415 | 28.749 | 9.271 |
| ATOM | SD | MET | A | 104 | 41.163 | 29.814 | 10.015 |
| ATOM | CE | MET | A | 104 | 39.763 | 28.689 | 10.090 |
| ATOM | O | MET | A | 104 | 45.128 | 29.530 | 7.474 |
| ATOM | CA | ASN | A | 105 | 47.201 | 27.909 | 6.482 |
| ATOM | CG2 | ILE | A | 107 | 44.710 | 34.237 | 8.071 |
| ATOM | CD1 | ILE | A | 107 | 42.279 | 32.546 | 7.638 |
| ATOM | O | ILE | A | 107 | 47.536 | 34.661 | 6.821 |
| ATOM | CA | ALA | A | 108 | 49.252 | 32.809 | 7.921 |
| ATOM | CB | ALA | A | 108 | 49.613 | 31.745 | 8.959 |
| ATOM | O | ALA | A | 108 | 51.357 | 33.582 | 7.076 |
| ATOM | N | LYS | A | 114 | 50.989 | 40.121 | 4.422 |
| ATOM | CB | LYS | A | 114 | 49.659 | 39.422 | 6.349 |
| ATOM | CD | LYS | A | 114 | 50.479 | 37.681 | 7.965 |
| ATOM | CE | LYS | A | 114 | 51.122 | 36.318 | 8.106 |
| ATOM | NZ | LYS | A | 114 | 52.403 | 36.271 | 7.345 |
| ATOM | N | ALA | A | 115 | 49.121 | 42.363 | 4.988 |
| ATOM | CA | ALA | A | 115 | 48.224 | 43.514 | 4.993 |
| ATOM | CB | ALA | A | 115 | 49.021 | 44.816 | 5.065 |
| ATOM | CG | GLU | A | 118 | 45.071 | 40.454 | 7.074 |
| ATOM | OE2 | GLU | A | 118 | 44.218 | 38.267 | 7.520 |
| ATOM | CD2 | HIS | A | 132 | 34.060 | 36.247 | 15.526 |
| ATOM | CE1 | HIS | A | 132 | 35.771 | 35.402 | 14.447 |
| ATOM | NE2 | HIS | A | 132 | 35.322 | 35.720 | 15.649 |
| ATOM | O | HIS | A | 132 | 34.836 | 39.095 | 12.675 |
| ATOM | SG | CYS | A | 133 | 34.765 | 35.332 | 9.372 |
| ATOM | CG1 | VAL | A | 135 | 35.077 | 43.110 | 14.983 |
| ATOM | CA | ALA | A | 136 | 38.186 | 40.749 | 12.941 |
| ATOM | CB | ALA | A | 136 | 38.197 | 39.238 | 12.924 |
| ATOM | O | ALA | A | 136 | 40.246 | 41.806 | 12.333 |
| ATOM | ND1 | HIS | A | 137 | 40.517 | 38.915 | 8.235 |
| ATOM | CE1 | HIS | A | 137 | 40.229 | 37.629 | 8.163 |
| ATOM | NE2 | HIS | A | 137 | 38.923 | 37.502 | 8.009 |
| ATOM | CB | HIS | A | 139 | 40.410 | 44.362 | 14.769 |
| ATOM | CG | HIS | A | 139 | 41.301 | 44.548 | 15.963 |
| ATOM | CD2 | HIS | A | 139 | 42.219 | 43.729 | 16.529 |
| ATOM | CE1 | HIS | A | 139 | 42.194 | 45.593 | 17.687 |
| ATOM | NE2 | HIS | A | 139 | 42.759 | 44.403 | 17.601 |
| ATOM | OG1 | THR | A | 140 | 43.391 | 41.000 | 12.322 |
| ATOM | CG2 | THR | A | 140 | 45.224 | 41.726 | 10.943 |
| ATOM | CB | THR | A | 143 | 46.647 | 45.739 | 14.859 |
| ATOM | OG1 | THR | A | 143 | 46.606 | 44.614 | 13.978 |
| ATOM | CG2 | THR | A | 143 | 45.377 | 45.766 | 15.704 |
| ATOM | O | THR | A | 143 | 49.154 | 47.078 | 13.758 |
| ATOM | CA | VAL | A | 144 | 49.134 | 46.659 | 11.050 |
| ATOM | CB | VAL | A | 144 | 49.041 | 45.516 | 10.013 |
| ATOM | CG1 | VAL | A | 144 | 48.891 | 44.185 | 10.726 |
| ATOM | O | VAL | A | 144 | 50.259 | 48.007 | 9.412 |
